SEQUENCE LISTING (268 sequences)

SEQ ID NO: 1 - DNA, 83 bp, Artificial Sequence
  Synthetic Template 1
  modified_base (1)..(2): Biotin
  misc_feature (34)..(63): n is a, c, g, or t
aattttttttctctcttcctccctttttccttcnnnnnnnnnnnnnnnnnnnnnnnnnnnnnncaaggtaggtcggcgtcact

SEQ ID NO: 2 - DNA, 28 bp, Artificial Sequence
  Synthetic Primer 1
atatatatagtgacgccgacctaccttg

SEQ ID NO: 3 - DNA, 33 bp, Artificial Sequence
  Synthetic Primer 2
  modified_base (1)..(2): Biotin
aattttttttctctcttcctccctttttccttc

SEQ ID NO: 4 - DNA, 31 bp, Artificial Sequence
  Synthetic aptamer
  misc_feature (5)..(5): n is Tyr-dU
  misc_feature (7)..(7): n is Nap-dC
  misc_feature (8)..(8): n is Tyr-dU
  misc_feature (10)..(10): n is Tyr-dU
  misc_feature (12)..(12): n is Nap-dC
  misc_feature (14)..(14): n is Tyr-dU
  misc_feature (15)..(15): n is Nap-dC
  misc_feature (16)..(16): n is Nap-dC
  misc_feature (19)..(19): n is Nap-dC
  misc_feature (20)..(20): n is Nap-dC
  misc_feature (22)..(22): n is Nap-dC
  misc_feature (23)..(23): n is Tyr-dU
  misc_feature (24)..(24): n is Nap-dC
  misc_feature (28)..(28): n is Nap-dC
ggganannangngnnnganngnnnggangga

SEQ ID NO: 5 - DNA, 31 bp, Artificial Sequence
  Synthetic aptamer
  misc_feature: See specification as filed for detailed description of substitutions and preferred embodiments
  misc_feature (2)..(2): n is dA, dG or a C-5 modified pyrimidine nucleoside
  misc_feature (5)..(5): n is a C-5 modified pyrimidine nucleoside
  misc_feature (7)..(7): n is a C-5 modified pyrimidine nucleoside
  misc_feature (8)..(8): n is a C-5 modified pyrimidine nucleoside
  misc_feature (10)..(10): n is dA or a C-5 modified pyrimidine nucleoside
  misc_feature (12)..(12): n is a C-5 modified pyrimidine nucleoside
  misc_feature (14)..(14): n is a C-5 modified pyrimidine nucleoside
  misc_feature (15)..(15): n is a C-5 modified pyrimidine nucleoside
  misc_feature (16)..(16): n is dA or a C-5 modified pyrimidine nucleoside
  misc_feature (19)..(19): n is a C-5 modified pyrimidine nucleoside
  misc_feature (20)..(20): n is dA or a C-5 modified pyrimidine nucleoside
  misc_feature (22)..(22): n is a C-5 modified pyrimidine nucleoside
  misc_feature (23)..(23): n is a C-5 modified pyrimidine nucleoside
  misc_feature (24)..(24): n is a C-5 modified pyrimidine nucleoside
  misc_feature (28)..(28): n is a C-5 modified pyrimidine nucleoside
  misc_feature (29)..(29): n is dA, dG or a C-5 modified pyrimidine nucleoside
  misc_feature (30)..(30): n is dA, dG or a C-5 modified pyrimidine nucleoside
  misc_feature (31)..(31): n is dA, dG or a C-5 modified pyrimidine nucleoside
gnranrnnanrnrnnnrannrnnnrrannnn

SEQ ID NO: 6 - DNA, 40 bp, Artificial Sequence
  Synthetic 26874-29_4
  misc_feature (1)..(40): Every n is a C-5 modified pyrimidine such as Tyr-dU or Nap-dC - see specification as filed for detailed description of substitutions and preferred embodiments
ccttggganannangngnnnganngnnngganggagaagg

SEQ ID NO: 7 - DNA, 29 bp, Artificial Sequence
  Synthetic 26874-29_20
  misc_feature (1)..(29): Every n is a C-5 modified pyrimidine such as Tyr-dU or Nap-dC - see specification as filed for detailed description of substitutions and preferred embodiments
ggganannangngnnnganngnnnggang

SEQ ID NO: 8 - DNA, 38 bp, Artificial Sequence
  Synthetic 26874-29_6
  misc_feature (1)..(38): Every n is a C-5 modified pyrimidine such as Tyr-dU or Nap-dC - see specification as filed for detailed description of substitutions and preferred embodiments
ttggganannangngnnnganngnnngganggagaagg

SEQ ID NO: 9 - DNA, 36 bp, Artificial Sequence
  Synthetic 26874-29_7
  misc_feature (1)..(36): Every n is a C-5 modified pyrimidine such as Tyr-dU or Nap-dC - see specification as filed for detailed description of substitutions and preferred embodiments
ggganannangngnnnganngnnngganggagaagg

SEQ ID NO: 10 - DNA, 38 bp, Artificial Sequence
  Synthetic 26874-29_12
  misc_feature (1)..(38): Every n is a C-5 modified pyrimidine such as Tyr-dU or Nap-dC - see specification as filed for detailed description of substitutions and preferred embodiments
ccttggganannangngnnnganngnnngganggagaa

SEQ ID NO: 11 - DNA, 36 bp, Artificial Sequence
  Synthetic 26874-29_13
  misc_feature (1)..(36): Every n is a C-5 modified pyrimidine such as Tyr-dU or Nap-dC - see specification as filed for detailed description of substitutions and preferred embodiments
ccttggganannangngnnnganngnnngganggag

SEQ ID NO: 12 - DNA, 34 bp, Artificial Sequence
  Synthetic 26874-29_14
  misc_feature (1)..(34): Every n is a C-5 modified pyrimidine such as Tyr-dU or Nap-dC - see specification as filed for detailed description of substitutions and preferred embodiments
ccttggganannangngnnnganngnnnggangg

SEQ ID NO: 13 - DNA, 32 bp, Artificial Sequence
  Synthetic 26874-29_15
  misc_feature (1)..(32): Every n is a C-5 modified pyrimidine such as Tyr-dU or Nap-dC - see specification as filed for detailed description of substitutions and preferred embodiments
ccttggganannangngnnnganngnnnggan

SEQ ID NO: 14 - DNA, 30 bp, Artificial Sequence
  Synthetic 26874-29_18
  misc_feature (1)..(30): Every n is a C-5 modified pyrimidine such as Tyr-dU or Nap-dC - see specification as filed for detailed description of substitutions and preferred embodiments
ggganannangngnnnganngnnnggangg

SEQ ID NO: 15 - DNA, 29 bp, Artificial Sequence
  Synthetic 26874-29_19
  misc_feature (1)..(29): Every n is a C-5 modified pyrimidine such as Tyr-dU or Nap-dC - see specification as filed for detailed description of substitutions and preferred embodiments
gganannangngnnnganngnnnggangg

SEQ ID NO: 16 - DNA, 28 bp, Artificial Sequence
  Synthetic 26874-29_21
  misc_feature (1)..(28): Every n is a C-5 modified pyrimidine such as Tyr-dU or Nap-dC - see specification as filed for detailed description of substitutions and preferred embodiments
gganannangngnnnganngnnnggang

SEQ ID NO: 17 - DNA, 26 bp, Artificial Sequence
  Synthetic 26874-29_22
  misc_feature (1)..(26): Every n is a C-5 modified pyrimidine such as Tyr-dU or Nap-dC - see specification as filed for detailed description of substitutions and preferred embodiments
nannangngnnnganngnnnggangg

SEQ ID NO: 18 - DNA, 25 bp, Artificial Sequence
  Synthetic 26874-29_23
  misc_feature (1)..(25): Every n is a C-5 modified pyrimidine such as Tyr-dU or Nap-dC - see specification as filed for detailed description of substitutions and preferred embodiments
annangngnnnganngnnnggangg

SEQ ID NO: 19 - DNA, 25 bp, Artificial Sequence
  Synthetic 26874-29_24
  misc_feature (1)..(25): Every n is a C-5 modified pyrimidine such as Tyr-dU or Nap-dC - see specification as filed for detailed description of substitutions and preferred embodiments
nannangngnnnganngnnnggang

SEQ ID NO: 20 - DNA, 24 bp, Artificial Sequence
  Synthetic 26874-29_25
  misc_feature (1)..(24): Every n is a C-5 modified pyrimidine such as Tyr-dU or Nap-dC - see specification as filed for detailed description of substitutions and preferred embodiments
annangngnnnganngnnnggang

SEQ ID NO: 21 - protein, 223 aa, SARS-CoV-2
  misc_feature (1)..(223): Spike RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 22 - protein, 666 aa, SARS-CoV-2
  misc_feature (1)..(666): Spike S1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 23 - protein, 1197 aa, SARS-CoV-2
  misc_feature (1)..(1197): Spike S1 & S2 ECD
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLDAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 24 - protein, 1135 aa, SARS-CoV-2
  misc_feature (1)..(1135): Spike S1 & S2 ECD stable trimer
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKE

SEQ ID NO: 25 - protein, 669 aa, SARS-CoV-2
  misc_feature (1)..(669): Spike S1 D614G
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSNNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 26 - protein, 223 aa, SARS-CoV-2
  misc_feature (1)..(223): Spike RBD N501Y
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 27 - protein, 219 aa, SARS-CoV-2
  misc_feature (1)..(219): Spike RBD E484K
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNK

SEQ ID NO: 28 - DNA, 29 bp, Artificial Sequence
  Synthetic 26874-29_46
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 29 - DNA, 29 bp, Artificial Sequence
  Synthetic 26874-29_47
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 30 - DNA, 29 bp, Artificial Sequence
  Synthetic 26874-29_48
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 31 - DNA, 29 bp, Artificial Sequence
  Synthetic 26874-29_49
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 32 - DNA, 29 bp, Artificial Sequence
  Synthetic 26874-29_50
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 33 - DNA, 29 bp, Artificial Sequence
  Synthetic 26874-29_51
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 34 - DNA, 29 bp, Artificial Sequence
  Synthetic 26874-29_52
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 35 - DNA, 29 bp, Artificial Sequence
  Synthetic 26874-29_53
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 36 - DNA, 29 bp, Artificial Sequence
  Synthetic 26874-29_54
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 37 - DNA, 29 bp, Artificial Sequence
  Synthetic 26874-29_55
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 38 - DNA, 29 bp, Artificial Sequence
  Synthetic 26874-29_56
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 39 - DNA, 29 bp, Artificial Sequence
  Synthetic 26874-29_57
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 40 - DNA, 29 bp, Artificial Sequence
  Synthetic 26874-29_58
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 41 - DNA, 29 bp, Artificial Sequence
  Synthetic 26874-29_59
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 42 - DNA, 29 bp, Artificial Sequence
  Synthetic 26874-29_60
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 43 - DNA, 29 bp, Artificial Sequence
  Synthetic 26874-29_61
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 44 - DNA, 29 bp, Artificial Sequence
  Synthetic 26874-29_62
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 45 - DNA, 29 bp, Artificial Sequence
  Synthetic 26874-29_63
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 46 - DNA, 29 bp, Artificial Sequence
  Synthetic 26874-29_64
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 47 - DNA, 29 bp, Artificial Sequence
  Synthetic 26874-29_65
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 48 - DNA, 29 bp, Artificial Sequence
  Synthetic 26874-29_66
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 49 - DNA, 29 bp, Artificial Sequence
  Synthetic 26874-29_68
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 50 - DNA, 29 bp, Artificial Sequence
  Synthetic 26874-29_70
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 51 - DNA, 29 bp, Artificial Sequence
  Synthetic 26874-29_72
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 52 - DNA, 29 bp, Artificial Sequence
  Synthetic 26874-29_74
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 53 - DNA, 29 bp, Artificial Sequence
  Synthetic 26874-29_76
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 54 - DNA, 29 bp, Artificial Sequence
  Synthetic 26874-29_77
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 55 - DNA, 29 bp, Artificial Sequence
  Synthetic 26874-29_78
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 56 - DNA, 29 bp, Artificial Sequence
  Synthetic 26874-29_79
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 57 - DNA, 29 bp, Artificial Sequence
  Synthetic 26874-29_80
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 58 - DNA, 29 bp, Artificial Sequence
  Synthetic 26874-29_81
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 59 - DNA, 29 bp, Artificial Sequence
  Synthetic 26874-29_82
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 60 - DNA, 29 bp, Artificial Sequence
  Synthetic 26874-29_83
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 61 - DNA, 29 bp, Artificial Sequence
  Synthetic 26874-29_84
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 62 - DNA, 29 bp, Artificial Sequence
  Synthetic 26874-29_85
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 63 - DNA, 29 bp, Artificial Sequence
  Synthetic 26874-29_86
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 64 - DNA, 29 bp, Artificial Sequence
  Synthetic 26874-29_87
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 65 - DNA, 29 bp, Artificial Sequence
  Synthetic 26874-29_88
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 66 - DNA, 29 bp, Artificial Sequence
  Synthetic 26874-29_89
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 67 - DNA, 29 bp, Artificial Sequence
  Synthetic 26874-29_90
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 68 - DNA, 29 bp, Artificial Sequence
  Synthetic 26874-29_91
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 69 - DNA, 29 bp, Artificial Sequence
  Synthetic 26874-29_92
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 70 - DNA, 29 bp, Artificial Sequence
  Synthetic 26874-29_93
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 71 - DNA, 29 bp, Artificial Sequence
  Synthetic 26874-29_94
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 72 - DNA, 29 bp, Artificial Sequence
  Synthetic 26874-29_95
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 73 - DNA, 29 bp, Artificial Sequence
  Synthetic 26874-29_96
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
gggahannangngnnnganngnnnggang

SEQ ID NO: 74 - DNA, 29 bp, Artificial Sequence
  Synthetic 26874-29_97
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 75 - DNA, 29 bp, Artificial Sequence
  Synthetic 26874-29_98
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
gggananhangngnnnganngnnnggang

SEQ ID NO: 76 - DNA, 29 bp, Artificial Sequence
  Synthetic 26874-29_99
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 77 - DNA, 29 bp, Artificial Sequence
  Synthetic 26874-29_100
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangnghnnganngnnnggang

SEQ ID NO: 78 - DNA, 29 bp, Artificial Sequence
  Synthetic 26874-29_101
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 79 - DNA, 29 bp, Artificial Sequence
  Synthetic 26874-29_102
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangnghnnganngnnnggang

SEQ ID NO: 80 - DNA, 29 bp, Artificial Sequence
  Synthetic 26874-29_103
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 81 - DNA, 29 bp, Artificial Sequence
  Synthetic 26874-29_104
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnhnggang

SEQ ID NO: 82 - DNA, 29 bp, Artificial Sequence
  Synthetic 26874-29_105
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 83 - DNA, 29 bp, Artificial Sequence
  Synthetic 26874-29_106
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 84 - DNA, 29 bp, Artificial Sequence
  Synthetic 26874-29_107
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 85 - DNA, 29 bp, Artificial Sequence
  Synthetic 26874-29_108
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 86 - DNA, 29 bp, Artificial Sequence
  Synthetic 26874-29_109
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 87 - DNA, 29 bp, Artificial Sequence
  Synthetic 26874-29_110
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 88 - DNA, 29 bp, Artificial Sequence
  Synthetic 26874-29_112
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 89 - DNA, 29 bp, Artificial Sequence
  Synthetic 26874-29_113
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 90 - DNA, 29 bp, Artificial Sequence
  Synthetic 26874-29_114
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 91 - DNA, 29 bp, Artificial Sequence
  Synthetic 26874-29_115
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 92 - DNA, 29 bp, Artificial Sequence
  Synthetic 26874-29_116
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 93 - DNA, 29 bp, Artificial Sequence
  Synthetic 26874-29_117
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 94 - DNA, 29 bp, Artificial Sequence
  Synthetic 26874-29_118
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 95 - DNA, 29 bp, Artificial Sequence
  Synthetic 26874-29_119
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 96 - DNA, 29 bp, Artificial Sequence
  Synthetic 26874-29_120
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 97 - DNA, 29 bp, Artificial Sequence
  Synthetic 26874-29_121
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 98 - DNA, 29 bp, Artificial Sequence
  Synthetic 26874-29_122
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 99 - DNA, 29 bp, Artificial Sequence
  Synthetic 26874-29_123
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 100 - DNA, 29 bp, Artificial Sequence
  Synthetic 26874-29_124
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 101 - DNA, 29 bp, Artificial Sequence
  Synthetic 26874-29_125
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 102 - DNA, 29 bp, Artificial Sequence
  Synthetic 26874-29_126
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 103 - DNA, 29 bp, Artificial Sequence
  Synthetic 26874-29_127
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
gggacannangngnnnganngnnnggang

SEQ ID NO: 104 - DNA, 29 bp, Artificial Sequence
  Synthetic 26874-29_128
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 105 - DNA, 29 bp, Artificial Sequence
  Synthetic 26874-29_130
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 106 - DNA, 29 bp, Artificial Sequence
  Synthetic 26874-29_132
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 107 - DNA, 29 bp, Artificial Sequence
  Synthetic 26874-29_134
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 108 - DNA, 29 bp, Artificial Sequence
  Synthetic 26874-29_136
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 109 - DNA, 29 bp, Artificial Sequence
  Synthetic 26874-29_138
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 110 - DNA, 29 bp, Artificial Sequence
  Synthetic 26874-29_140
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 111 - DNA, 29 bp, Artificial Sequence
  Synthetic 26874-29_142
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 112 - DNA, 29 bp, Artificial Sequence
  Synthetic 26874-29_144
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 113 - DNA, 29 bp, Artificial Sequence
  Synthetic 26874-29_146
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 114 - DNA, 29 bp, Artificial Sequence
  Synthetic 26874-29_147
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 115 - DNA, 29 bp, Artificial Sequence
  Synthetic 26874-29_148
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 116 - DNA, 29 bp, Artificial Sequence
  Synthetic 26874-29_149
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 117 - DNA, 29 bp, Artificial Sequence
  Synthetic 26874-29_150
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 118 - DNA, 29 bp, Artificial Sequence
  Synthetic 26874-29_151
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 119 - DNA, 29 bp, Artificial Sequence
  Synthetic 26874-29_152
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 120 - DNA, 29 bp, Artificial Sequence
  Synthetic 26874-29_153
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 121 - DNA, 29 bp, Artificial Sequence
  Synthetic 26874-29_154
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 122 - DNA, 29 bp, Artificial Sequence
  Synthetic 26874-29_155
  misc_feature (1)..(29): Every n is a C-5 modified pyrimidine such as TyrdU, NapdC, ThdU, O, MOEdU, 2NapdU, BTdU, POPdU, 2NEdU, iBudU, MBndU, BPEdU, FBndU, NapdU, TrpdU, BndU, PBndU, NEdU, PEdU, BFdU, or PPdU
  misc_feature (1)..(29): See specification as filed for detailed description of substitutions and preferred embodiments
ggganannangngnnnganngnnnggang

SEQ ID NO: 123 - protein, 1270 aa, SARS-CoV-2
  misc_feature (1)..(1270): Spike S1 & S2 ECD B1.1.7 variant (Alpha)
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQPYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVANNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 124 - protein, 1270 aa, SARS-CoV-2
  misc_feature (1)..(1270): Spike S1 & S2 ECD B1.351 variant (Beta)
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 125 - protein, 1273 aa, SARS-CoV-2
  misc_feature (1)..(1273): Spike S1 & S2 ECD P.1 variant (Gamma)
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 126 - protein, 1271 aa, SARS-CoV-2
  misc_feature (1)..(1271): Spike S1 & S2 ECD B.1.617.2 variant (Delta)
MFVFLVLLPLVSSQCVNLRTRTQLPPAYTNSFTRGVYYPDKVFRSSVLHSTQDLFLPFFSNVTWFHAIHFSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 127 - protein, 1269 aa, SARS-CoV-2
  misc_feature (1)..(1269): Spike S1 & S2 ECD B.1.1.529 variant (Omicron)
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 128 - DNA, 29 bp, Artificial Sequence
  Synthetic OH-26874-29_181
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 129 - DNA, 29 bp, Artificial Sequence
  Synthetic OH-26874-29_182
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 130 - DNA, 29 bp, Artificial Sequence
  Synthetic OH-26874-29_183
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 131 - DNA, 29 bp, Artificial Sequence
  Synthetic OH-26874-29_184
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 132 - DNA, 29 bp, Artificial Sequence
  Synthetic OH-26874-29_185
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 133 - DNA, 29 bp, Artificial Sequence
  Synthetic OH-26874-29_186
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 134 - DNA, 29 bp, Artificial Sequence
  Synthetic OH-26874-29_187
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 135 - DNA, 29 bp, Artificial Sequence
  Synthetic OH-26874-29_188
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 136 - DNA, 29 bp, Artificial Sequence
  Synthetic OH-26874-29_189
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 137 - DNA, 29 bp, Artificial Sequence
  Synthetic OH-26874-29_190
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganarnangngnnnganngnnnggang

SEQ ID NO: 138 - DNA, 29 bp, Artificial Sequence
  Synthetic OH-26874-29_191
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangrgnnnganngnnnggang

SEQ ID NO: 139 - DNA, 29 bp, Artificial Sequence
  Synthetic OH-26874-29_192
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnrnganngnnnggang

SEQ ID NO: 140 - DNA, 29 bp, Artificial Sequence
  Synthetic OH-26874-29_193
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnrganngnnnggang

SEQ ID NO: 141 - DNA, 29 bp, Artificial Sequence
  Synthetic OH-26874-29_194
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnngarngnnnggang

SEQ ID NO: 142 - DNA, 29 bp, Artificial Sequence
  Synthetic OH-26874-29_195
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganrgnnnggang

SEQ ID NO: 143 - DNA, 29 bp, Artificial Sequence
  Synthetic OH-26874-29_196
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngrnnggang

SEQ ID NO: 144 - DNA, 29 bp, Artificial Sequence
  Synthetic OH-26874-29_197
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnrggang

SEQ ID NO: 145 - DNA, 29 bp, Artificial Sequence
  Synthetic OH-26874-29_198
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggarg

SEQ ID NO: 146 - DNA, 29 bp, Artificial Sequence
  Synthetic OH-26874-29_199
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 147 - DNA, 29 bp, Artificial Sequence
  Synthetic OH-26874-29_200
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 148 - DNA, 29 bp, Artificial Sequence
  Synthetic OH-26874-29_201
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 149 - DNA, 29 bp, Artificial Sequence
  Synthetic OH-26874-29_202
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 150 - DNA, 29 bp, Artificial Sequence
  Synthetic OH-26874-29_203
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 151 - DNA, 29 bp, Artificial Sequence
  Synthetic OH-26874-29_204
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 152 - DNA, 29 bp, Artificial Sequence
  Synthetic OH-26874-29_205
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 153 - DNA, 29 bp, Artificial Sequence
  Synthetic OH-26874-29_206
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 154 - DNA, 29 bp, Artificial Sequence
  Synthetic OH-26874-29_207
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 155 - DNA, 29 bp, Artificial Sequence
  Synthetic OH-26874-29_208
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 156 - DNA, 29 bp, Artificial Sequence
  Synthetic OH-26874-29_209
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 157 - DNA, 29 bp, Artificial Sequence
  Synthetic OH-26874-29_210
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 158 - DNA, 29 bp, Artificial Sequence
  Synthetic OH-26874-29_211
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 159 - DNA, 29 bp, Artificial Sequence
  Synthetic OH-26874-29_212
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 160 - DNA, 29 bp, Artificial Sequence
  Synthetic OH-26874-29_213
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 161 - DNA, 29 bp, Artificial Sequence
  Synthetic OH-26874-29_214
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 162 - DNA, 29 bp, Artificial Sequence
  Synthetic OH-26874-29_215
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 163 - DNA, 29 bp, Artificial Sequence
  Synthetic OH-26874-29_216
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 164 - DNA, 29 bp, Artificial Sequence
  Synthetic OH-26874-29_314
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 165 - DNA, 29 bp, Artificial Sequence
  Synthetic OH-26874-29_315
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 166 - DNA, 29 bp, Artificial Sequence
  Synthetic OH-26874-29_316
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 167 - DNA, 29 bp, Artificial Sequence
  Synthetic OH-26874-29_317
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 168 - DNA, 29 bp, Artificial Sequence
  Synthetic OH-26874-29_318
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 169 - DNA, 30 bp, Artificial Sequence
  Synthetic OH-26874-29_319
  misc_feature (1)..(30): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(30): See specification as filed for detailed description of substitutions and preferred embodiments
ggganannangngnnngannngnnnggang

SEQ ID NO: 170 - DNA, 29 bp, Artificial Sequence
  Synthetic OH-26874-29_320
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 171 - DNA, 29 bp, Artificial Sequence
  Synthetic OH-26874-29_321
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 172 - DNA, 29 bp, Artificial Sequence
  Synthetic OH-26874-29_322
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 173 - DNA, 29 bp, Artificial Sequence
  Synthetic OH-26874-29_323
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 174 - DNA, 29 bp, Artificial Sequence
  Synthetic OH-26874-29_324
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 175 - DNA, 29 bp, Artificial Sequence
  Synthetic OH-26874-29_325
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 176 - DNA, 29 bp, Artificial Sequence
  Synthetic OH-26874-29_326
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 177 - DNA, 29 bp, Artificial Sequence
  Synthetic OH-26874-29_327
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 178 - DNA, 29 bp, Artificial Sequence
  Synthetic OH-26874-29_328
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 179 - DNA, 29 bp, Artificial Sequence
  Synthetic OH-26874-29_329
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 180 - DNA, 29 bp, Artificial Sequence
  Synthetic OH-26874-29_330
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 181 - DNA, 29 bp, Artificial Sequence
  Synthetic OH-26874-29_331
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 182 - DNA, 29 bp, Artificial Sequence
  Synthetic OH-26874-29_332
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 183 - DNA, 29 bp, Artificial Sequence
  Synthetic OH-26874-29_333
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 184 - DNA, 29 bp, Artificial Sequence
  Synthetic OH-26874-29_334
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 185 - DNA, 29 bp, Artificial Sequence
  Synthetic OH-26874-29_335
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 186 - DNA, 29 bp, Artificial Sequence
  Synthetic OH-26874-29_336
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 187 - DNA, 29 bp, Artificial Sequence
  Synthetic OH-26874-29_337
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 188 - DNA, 29 bp, Artificial Sequence
  Synthetic OH-26874-29_338
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 189 - DNA, 29 bp, Artificial Sequence
  Synthetic OH-26874-29_339
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 190 - DNA, 29 bp, Artificial Sequence
  Synthetic OH-26874-29_340
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
ggganannangngnnnganngnnnggang

SEQ ID NO: 191 - RNA, 29 nt, Artificial Sequence
  Synthetic OH-26874-29_354
  misc_feature (1)..(29): Every n is a C-5 modified pyrimidine such as Tyr-dU, PP-dC, Bn-dC, Nap-dC, 2Nap-dC, Tyr-dC, PBn-dC, DPP-dC, and DCPE-dC
  misc_feature (1)..(29): See specification as filed for detailed description of substitutions and preferred embodiments
gggauacuaugcguccgaccgcucggacg

SEQ ID NO: 192 - DNA, 50 bp, Artificial Sequence
  Synthetic 26860-16_3
  misc_feature (1)..(50): Every n is a C-5 modified pyrimidine such as Tyr-dU or Nap-dC - see specification as filed for detailed description of substitutions and preferred embodiments
gcatcggaangaangncngccnngcacnggngcggnccganngngaggga

SEQ ID NO: 193 - DNA, 48 bp, Artificial Sequence
  Synthetic 26860-16_5
  misc_feature (1)..(48): Every n is a C-5 modified pyrimidine such as Tyr-dU or Nap-dC - see specification as filed for detailed description of substitutions and preferred embodiments
atcggaangaangncngccnngcacnggngcggnccganngngaggga

SEQ ID NO: 194 - DNA, 46 bp, Artificial Sequence
  Synthetic 26860-16_6
  misc_feature (1)..(46): Every n is a C-5 modified pyrimidine such as Tyr-dU or Nap-dC - see specification as filed for detailed description of substitutions and preferred embodiments
cggaangaangncngccnngcacnggngcggnccganngngaggga

SEQ ID NO: 195 - DNA, 44 bp, Artificial Sequence
  Synthetic 26860-16_7
  misc_feature (1)..(44): Every n is a C-5 modified pyrimidine such as Tyr-dU or Nap-dC - see specification as filed for detailed description of substitutions and preferred embodiments
gaangaangncngccnngcacnggngcggnccganngngaggga

SEQ ID NO: 196 - DNA, 46 bp, Artificial Sequence
  Synthetic 26860-16_11
  misc_feature (1)..(46): Every n is a C-5 modified pyrimidine such as Tyr-dU or Nap-dC - see specification as filed for detailed description of substitutions and preferred embodiments
gcatcggaangaangncngccnngcacnggngcggnccganngnga

SEQ ID NO: 197 - DNA, 44 bp, Artificial Sequence
  Synthetic 26860-16_12
  misc_feature (1)..(44): Every n is a C-5 modified pyrimidine such as Tyr-dU or Nap-dC - see specification as filed for detailed description of substitutions and preferred embodiments
gcatcggaangaangncngccnngcacnggngcggnccganngn

SEQ ID NO: 198 - DNA, 42 bp, Artificial Sequence
  Synthetic 26860-16_13
  misc_feature (1)..(42): Every n is a C-5 modified pyrimidine such as Tyr-dU or Nap-dC - see specification as filed for detailed description of substitutions and preferred embodiments
gcatcggaangaangncngccnngcacnggngcggnccgann

SEQ ID NO: 199 - DNA, 40 bp, Artificial Sequence
  Synthetic 26860-16_14
  misc_feature (1)..(40): Every n is a C-5 modified pyrimidine such as Tyr-dU or Nap-dC - see specification as filed for detailed description of substitutions and preferred embodiments
gcatcggaangaangncngccnngcacnggngcggnccga

SEQ ID NO: 200 - DNA, 38 bp, Artificial Sequence
  Synthetic 26860-16_15
  misc_feature (1)..(38): Every n is a C-5 modified pyrimidine such as Tyr-dU or Nap-dC - see specification as filed for detailed description of substitutions and preferred embodiments
atcggaangaangncngccnngcacnggngcggnccga

SEQ ID NO: 201 - DNA, 37 bp, Artificial Sequence
  Synthetic 26860-16_16
  misc_feature (1)..(37): Every n is a C-5 modified pyrimidine such as Tyr-dU or Nap-dC - see specification as filed for detailed description of substitutions and preferred embodiments
tcggaangaangncngccnngcacnggngcggnccga

SEQ ID NO: 202 - DNA, 36 bp, Artificial Sequence
  Synthetic 26860-16_17
  misc_feature (1)..(36): Every n is a C-5 modified pyrimidine such as Tyr-dU or Nap-dC - see specification as filed for detailed description of substitutions and preferred embodiments
atcggaangaangncngccnngcacnggngcggncc

SEQ ID NO: 203 - DNA, 35 bp, Artificial Sequence
  Synthetic 26860-16_18
  misc_feature (1)..(35): Every n is a C-5 modified pyrimidine such as Tyr-dU or Nap-dC - see specification as filed for detailed description of substitutions and preferred embodiments
tcggaangaangncngccnngcacnggngcggncc

SEQ ID NO: 204 - DNA, 34 bp, Artificial Sequence
  Synthetic 26860-16_19
  misc_feature (1)..(34): Every n is a C-5 modified pyrimidine such as Tyr-dU or Nap-dC - see specification as filed for detailed description of substitutions and preferred embodiments
atcggaangaangncngccnngcacnggngcggn

SEQ ID NO: 205 - DNA, 33 bp, Artificial Sequence
  Synthetic 26860-16_20
  misc_feature (1)..(33): Every n is a C-5 modified pyrimidine such as Tyr-dU or Nap-dC - see specification as filed for detailed description of substitutions and preferred embodiments
tcggaangaangncngccnngcacnggngcggn

SEQ ID NO: 206 - DNA, 35 bp, Artificial Sequence
  Synthetic 26860-16_28
  misc_feature (1)..(35): Every n is a C-5 modified pyrimidine such as Tyr-dU or Nap-dC - see specification as filed for detailed description of substitutions and preferred embodiments
tcggaatgaatgtctgccttgcactggtgcggtcc

SEQ ID NO: 207 - DNA, 50 bp, Artificial Sequence
  Synthetic 26860-75_3
  misc_feature (1)..(50): Every n is a C-5 modified pyrimidine such as Tyr-dU or Nap-dC - see specification as filed for detailed description of substitutions and preferred embodiments
gcatcancncccagcnaangagngncngaannggacnggnccncngggga

SEQ ID NO: 208 - DNA, 48 bp, Artificial Sequence
  Synthetic 26860-75_5
  misc_feature (1)..(48): Every n is a C-5 modified pyrimidine such as Tyr-dU or Nap-dC - see specification as filed for detailed description of substitutions and preferred embodiments
atcancncccagcnaangagngncngaannggacnggnccncngggga

SEQ ID NO: 209 - DNA, 46 bp, Artificial Sequence
  Synthetic 26860-75_6
  misc_feature (1)..(46): Every n is a C-5 modified pyrimidine such as Tyr-dU or Nap-dC - see specification as filed for detailed description of substitutions and preferred embodiments
cancncccagcnaangagngncngaannggacnggnccncngggga

SEQ ID NO: 210 - DNA, 44 bp, Artificial Sequence
  Synthetic 26860-75_7
  misc_feature (1)..(44): Every n is a C-5 modified pyrimidine such as Tyr-dU or Nap-dC - see specification as filed for detailed description of substitutions and preferred embodiments
ncncccagcnaangagngncngaannggacnggnccncngggga

SEQ ID NO: 211 - DNA, 42 bp, Artificial Sequence
  Synthetic 26860-75_8
  misc_feature (1)..(42): Every n is a C-5 modified pyrimidine such as Tyr-dU or Nap-dC - see specification as filed for detailed description of substitutions and preferred embodiments
ncccagcnaangagngncngaannggacnggnccncngggga

SEQ ID NO: 212 - DNA, 40 bp, Artificial Sequence
  Synthetic 26860-75_9
  misc_feature (1)..(40): Every n is a C-5 modified pyrimidine such as Tyr-dU or Nap-dC - see specification as filed for detailed description of substitutions and preferred embodiments
ccagcnaangagngncngaannggacnggnccncngggga

SEQ ID NO: 213 - DNA, 48 bp, Artificial Sequence
  Synthetic 26860-75_10
  misc_feature (1)..(48): Every n is a C-5 modified pyrimidine such as Tyr-dU or Nap-dC - see specification as filed for detailed description of substitutions and preferred embodiments
gcatcancncccagcnaangagngncngaannggacnggnccncnggg

SEQ ID NO: 214 - DNA, 46 bp, Artificial Sequence
  Synthetic 26860-75_11
  misc_feature (1)..(46): Every n is a C-5 modified pyrimidine such as Tyr-dU or Nap-dC - see specification as filed for detailed description of substitutions and preferred embodiments
gcatcancncccagcnaangagngncngaannggacnggnccncng

SEQ ID NO: 215 - DNA, 40 bp, Artificial Sequence
  Synthetic 26860-75_14
  misc_feature (1)..(40): Every n is a C-5 modified pyrimidine such as Tyr-dU or Nap-dC - see specification as filed for detailed description of substitutions and preferred embodiments
gcatcancncccagcnaangagngncngaannggacnggn

SEQ ID NO: 216 - DNA, 46 bp, Artificial Sequence
  Synthetic 26860-75_15
  misc_feature (1)..(46): Every n is a C-5 modified pyrimidine such as Tyr-dU or Nap-dC - see specification as filed for detailed description of substitutions and preferred embodiments
atcancncccagcnaangagngncngaannggacnggnccncnggg

SEQ ID NO: 217 - DNA, 45 bp, Artificial Sequence
  Synthetic 26860-75_16
  misc_feature (1)..(45): Every n is a C-5 modified pyrimidine such as Tyr-dU or Nap-dC - see specification as filed for detailed description of substitutions and preferred embodiments
tcancncccagcnaangagngncngaannggacnggnccncnggg

SEQ ID NO: 218 - DNA, 45 bp, Artificial Sequence
  Synthetic 26860-75_17
  misc_feature (1)..(45): Every n is a C-5 modified pyrimidine such as Tyr-dU or Nap-dC - see specification as filed for detailed description of substitutions and preferred embodiments
atcancncccagcnaangagngncngaannggacnggnccncngg

SEQ ID NO: 219 - DNA, 44 bp, Artificial Sequence
  Synthetic 26860-75_18
  misc_feature (1)..(44): Every n is a C-5 modified pyrimidine such as Tyr-dU or Nap-dC - see specification as filed for detailed description of substitutions and preferred embodiments
tcancncccagcnaangagngncngaannggacnggnccncngg

SEQ ID NO: 220 - DNA, 44 bp, Artificial Sequence
  Synthetic 26860-75_26
  misc_feature (1)..(44): Every n is a C-5 modified pyrimidine such as Tyr-dU or Nap-dC - see specification as filed for detailed description of substitutions and preferred embodiments
tcatctcccagctaatgagtgtctgaattggactggtcctctgg

SEQ ID NO: 221 - DNA, 40 bp, Artificial Sequence
  Synthetic 26876-3_4
  misc_feature (1)..(40): Every n is a C-5 modified pyrimidine such as Tyr-dU or Nap-dC - see specification as filed for detailed description of substitutions and preferred embodiments
ccttgnaggnggnananggnannnnananngagnggaagg

SEQ ID NO: 222 - DNA, 38 bp, Artificial Sequence
  Synthetic 26876-3_5
  misc_feature (1)..(38): Every n is a C-5 modified pyrimidine such as Tyr-dU or Nap-dC - see specification as filed for detailed description of substitutions and preferred embodiments
ttgnaggnggnananggnannnnananngagnggaagg

SEQ ID NO: 223 - DNA, 36 bp, Artificial Sequence
  Synthetic 26876-3_6
  misc_feature (1)..(36): Every n is a C-5 modified pyrimidine such as Tyr-dU or Nap-dC - see specification as filed for detailed description of substitutions and preferred embodiments
gnaggnggnananggnannnnananngagnggaagg

SEQ ID NO: 224 - DNA, 34 bp, Artificial Sequence
  Synthetic 26876-3_7
  misc_feature (1)..(34): Every n is a C-5 modified pyrimidine such as Tyr-dU or Nap-dC - see specification as filed for detailed description of substitutions and preferred embodiments
aggnggnananggnannnnananngagnggaagg

SEQ ID NO: 225 - DNA, 32 bp, Artificial Sequence
  Synthetic 26876-3_8
  misc_feature (1)..(32): Every n is a C-5 modified pyrimidine such as Tyr-dU or Nap-dC - see specification as filed for detailed description of substitutions and preferred embodiments
gnggnananggnannnnananngagnggaagg

SEQ ID NO: 226 - DNA, 30 bp, Artificial Sequence
  Synthetic 26876-3_9
  misc_feature (1)..(30): Every n is a C-5 modified pyrimidine such as Tyr-dU or Nap-dC - see specification as filed for detailed description of substitutions and preferred embodiments
ggnananggnannnnananngagnggaagg

SEQ ID NO: 227 - DNA, 38 bp, Artificial Sequence
  Synthetic 26876-3_10
  misc_feature (1)..(38): Every n is a C-5 modified pyrimidine such as Tyr-dU or Nap-dC - see specification as filed for detailed description of substitutions and preferred embodiments
ccttgnaggnggnananggnannnnananngagnggaa

SEQ ID NO: 228 - DNA, 36 bp, Artificial Sequence
  Synthetic 26876-3_11
  misc_feature (1)..(36): Every n is a C-5 modified pyrimidine such as Tyr-dU or Nap-dC - see specification as filed for detailed description of substitutions and preferred embodiments
ccttgnaggnggnananggnannnnananngagngg

SEQ ID NO: 229 - DNA, 34 bp, Artificial Sequence
  Synthetic 26876-3_12
  misc_feature (1)..(34): Every n is a C-5 modified pyrimidine such as Tyr-dU or Nap-dC - see specification as filed for detailed description of substitutions and preferred embodiments
ccttgnaggnggnananggnannnnananngagn

SEQ ID NO: 230 - DNA, 32 bp, Artificial Sequence
  Synthetic 26876-3_13
  misc_feature (1)..(32): Every n is a C-5 modified pyrimidine such as Tyr-dU or Nap-dC - see specification as filed for detailed description of substitutions and preferred embodiments
ccttgnaggnggnananggnannnnanannga

SEQ ID NO: 231 - DNA, 30 bp, Artificial Sequence
  Synthetic 26876-3_14
  misc_feature (1)..(30): Every n is a C-5 modified pyrimidine such as Tyr-dU or Nap-dC - see specification as filed for detailed description of substitutions and preferred embodiments
ccttgnaggnggnananggnannnnanann

SEQ ID NO: 232 - DNA, 30 bp, Artificial Sequence
  Synthetic 26876-3_15
  misc_feature (1)..(30): Every n is a C-5 modified pyrimidine such as Tyr-dU or Nap-dC - see specification as filed for detailed description of substitutions and preferred embodiments
aggnggnananggnannnnananngagngg

SEQ ID NO: 233 - DNA, 29 bp, Artificial Sequence
  Synthetic 26876-3_16
  misc_feature (1)..(29): Every n is a C-5 modified pyrimidine such as Tyr-dU or Nap-dC - see specification as filed for detailed description of substitutions and preferred embodiments
aggnggnananggnannnnananngagng

SEQ ID NO: 234 - DNA, 29 bp, Artificial Sequence
  Synthetic 26876-3_17
  misc_feature (1)..(29): Every n is a C-5 modified pyrimidine such as Tyr-dU or Nap-dC - see specification as filed for detailed description of substitutions and preferred embodiments
ggnggnananggnannnnananngagngg

SEQ ID NO: 235 - DNA, 28 bp, Artificial Sequence
  Synthetic 26876-3_18
  misc_feature (1)..(28): Every n is a C-5 modified pyrimidine such as Tyr-dU or Nap-dC - see specification as filed for detailed description of substitutions and preferred embodiments
ggnggnananggnannnnananngagng

SEQ ID NO: 236 - DNA, 27 bp, Artificial Sequence
  Synthetic 26876-3_20
  misc_feature (1)..(27): Every n is a C-5 modified pyrimidine such as Tyr-dU or Nap-dC - see specification as filed for detailed description of substitutions and preferred embodiments
ggnggnananggnannnnananngagn

SEQ ID NO: 237 - DNA, 26 bp, Artificial Sequence
  Synthetic 26876-3_21
  misc_feature (1)..(26): Every n is a C-5 modified pyrimidine such as Tyr-dU or Nap-dC - see specification as filed for detailed description of substitutions and preferred embodiments
gnggnananggnannnnananngagn

SEQ ID NO: 238 - DNA, 28 bp, Artificial Sequence
  Synthetic 26876-3_36
  misc_feature (1)..(28): Every n is a C-5 modified pyrimidine such as Tyr-dU or Nap-dC - see specification as filed for detailed description of substitutions and preferred embodiments
ggcggcacatggcacttcatatcgagcg

SEQ ID NO: 239 - DNA, 40 bp, Artificial Sequence
  Synthetic 26876-13_4
  misc_feature (1)..(40): Every n is a C-5 modified pyrimidine such as Tyr-dU or Nap-dC - see specification as filed for detailed description of substitutions and preferred embodiments
ccttgngnaangnannnnannnggnnngaangngagaagg

SEQ ID NO: 240 - DNA, 38 bp, Artificial Sequence
  Synthetic 26876-13_5
  misc_feature (1)..(38): Every n is a C-5 modified pyrimidine such as Tyr-dU or Nap-dC - see specification as filed for detailed description of substitutions and preferred embodiments
ttgngnaangnannnnannnggnnngaangngagaagg

SEQ ID NO: 241 - DNA, 36 bp, Artificial Sequence
  Synthetic 26876-13_6
  misc_feature (1)..(36): Every n is a C-5 modified pyrimidine such as Tyr-dU or Nap-dC - see specification as filed for detailed description of substitutions and preferred embodiments
gngnaangnannnnannnggnnngaangngagaagg

SEQ ID NO: 242 - DNA, 38 bp, Artificial Sequence
  Synthetic 26876-13_10
  misc_feature (1)..(38): Every n is a C-5 modified pyrimidine such as Tyr-dU or Nap-dC - see specification as filed for detailed description of substitutions and preferred embodiments
ccttgngnaangnannnnannnggnnngaangngagaa

SEQ ID NO: 243 - DNA, 36 bp, Artificial Sequence
  Synthetic 26876-13_11
  misc_feature (1)..(36): Every n is a C-5 modified pyrimidine such as Tyr-dU or Nap-dC - see specification as filed for detailed description of substitutions and preferred embodiments
ccttgngnaangnannnnannnggnnngaangngag

SEQ ID NO: 244 - DNA, 34 bp, Artificial Sequence
  Synthetic 26876-13_12
  misc_feature (1)..(34): Every n is a C-5 modified pyrimidine such as Tyr-dU or Nap-dC - see specification as filed for detailed description of substitutions and preferred embodiments
ccttgngnaangnannnnannnggnnngaangng

SEQ ID NO: 245 - DNA, 32 bp, Artificial Sequence
  Synthetic 26876-13_13
  misc_feature (1)..(32): Every n is a C-5 modified pyrimidine such as Tyr-dU or Nap-dC - see specification as filed for detailed description of substitutions and preferred embodiments
ccttgngnaangnannnnannnggnnngaang

SEQ ID NO: 246 - DNA, 30 bp, Artificial Sequence
  Synthetic 26876-13_14
  misc_feature (1)..(30): Every n is a C-5 modified pyrimidine such as Tyr-dU or Nap-dC - see specification as filed for detailed description of substitutions and preferred embodiments
ccttgngnaangnannnnannnggnnngaa

SEQ ID NO: 247 - DNA, 30 bp, Artificial Sequence
  Synthetic 26876-13_15
  misc_feature (1)..(30): Every n is a C-5 modified pyrimidine such as Tyr-dU or Nap-dC - see specification as filed for detailed description of substitutions and preferred embodiments
gngnaangnannnnannnggnnngaangng

SEQ ID NO: 248 - DNA, 29 bp, Artificial Sequence
  Synthetic 26876-13_16
  misc_feature (1)..(29): Every n is a C-5 modified pyrimidine such as Tyr-dU or Nap-dC - see specification as filed for detailed description of substitutions and preferred embodiments
ngnaangnannnnannnggnnngaangng

SEQ ID NO: 249 - DNA, 29 bp, Artificial Sequence
  Synthetic 26876-13_17
  misc_feature (1)..(29): Every n is a C-5 modified pyrimidine such as Tyr-dU or Nap-dC - see specification as filed for detailed description of substitutions and preferred embodiments
gngnaangnannnnannnggnnngaangn

SEQ ID NO: 250 - DNA, 28 bp, Artificial Sequence
  Synthetic 26876-13_18
  misc_feature (1)..(28): Every n is a C-5 modified pyrimidine such as Tyr-dU or Nap-dC - see specification as filed for detailed description of substitutions and preferred embodiments
ngnaangnannnnannnggnnngaangn

SEQ ID NO: 251 - DNA, 28 bp, Artificial Sequence
  Synthetic 26876-13_26
  misc_feature (1)..(28): Every n is a C-5 modified pyrimidine such as Tyr-dU or Nap-dC - see specification as filed for detailed description of substitutions and preferred embodiments
tgcaacgcaccttattcggcttgaatgt

SEQ ID NO: 252 - DNA, 28 bp, Artificial Sequence
  Synthetic aptamer
ggcggcacatggcacttcatatcgagcg

SEQ ID NO: 253 - DNA, 28 bp, Artificial Sequence
  Synthetic aptamer
  misc_feature: See specification as filed for detailed description of substitutions and preferred embodiments
  misc_feature (1)..(1): n is dA, dG or a C-5 modified pyrimidine nucleoside
  misc_feature (3)..(3): n is dA or a C-5 modified pyrimidine nucleoside
  misc_feature (6)..(6): n is a C-5 modified pyrimidine nucleoside
  misc_feature (7)..(7): n is is dA or the C-5 modified pyrimidine Tyr-dU
  misc_feature (8)..(8): n is a C-5 modified pyrimidine nucleoside
  misc_feature (9)..(9): n is is dA or the C-5 modified pyrimidine Tyr-dU
  misc_feature (10)..(10): n is a C-5 modified pyrimidine nucleoside
  misc_feature (13)..(13): n is a C-5 modified pyrimidine nucleoside
  misc_feature (14)..(14): n is is dA or the C-5 modified pyrimidine Tyr-dU
  misc_feature (15)..(15): n is a C-5 modified pyrimidine nucleoside
  misc_feature (16)..(16): n is a C-5 modified pyrimidine nucleoside
  misc_feature (17)..(17): n is a C-5 modified pyrimidine nucleoside
  misc_feature (18)..(18): n is a C-5 modified pyrimidine nucleoside
  misc_feature (19)..(19): n is dA, dG or a C-5 modified pyrimidine nucleoside
  misc_feature (20)..(20): n is is dA or the C-5 modified pyrimidine Tyr-dU
  misc_feature (21)..(21): n is dA, dG or a C-5 modified pyrimidine nucleoside
  misc_feature (22)..(22): n is a C-5 modified pyrimidine nucleoside
  misc_feature (23)..(23): n is a C-5 modified pyrimidine nucleoside
  misc_feature (27)..(27): n is dA or a C-5 modified pyrimidine nucleoside
nrnrrnnnnngrnnnnnnnnnnnrarnr

SEQ ID NO: 254 - DNA, 28 bp, Artificial Sequence
  Synthetic aptamer
tgcaacgcaccttattcggcttgaatgt

SEQ ID NO: 255 - DNA, 28 bp, Artificial Sequence
  Synthetic aptamer
  misc_feature: See specification as filed for detailed description of substitutions and preferred embodiments
  misc_feature (1)..(1): n is Tyr-dU
  misc_feature (3)..(3): n is Nap-dC or Tyr-dU
  misc_feature (4)..(4): n is dA, dG or Tyr-dU
  misc_feature (6)..(6): n is Nap-dC or Tyr-dU
  misc_feature (8)..(8): n is Nap-dC or Tyr-dU
  misc_feature (9)..(9): n is dA or Tyr-dU
  misc_feature (10)..(10): n is Nap-dC or Tyr-dU
  misc_feature (11)..(11): n is Nap-dC or Tyr-dU
  misc_feature (12)..(12): n is dA or Tyr-dU
  misc_feature (13)..(13): n is Tyr-dU
  misc_feature (14)..(14): n is dA or Tyr-dU
  misc_feature (15)..(15): n is Tyr-dU
  misc_feature (16)..(16): n is Tyr-dU
  misc_feature (17)..(17): n is dA, Nap-dC or Tyr-dU
  misc_feature (20)..(20): n is Nap-dC or Tyr-dU
  misc_feature (21)..(21): n is dA, Nap-dC or Tyr-dU
  misc_feature (22)..(22): n is Tyr-dU
  misc_feature (26)..(26): n is dA, Nap-dC, dG or Tyr-dU
  misc_feature (27)..(27): n is dA, dG or Tyr-dU
  misc_feature (28)..(28): n is Nap-dC, dG or Tyr-dU
nrnnrnrnnnnnnnnnnrrnnnrrrnnn

SEQ ID NO: 256 - DNA, 44 bp, Artificial Sequence; identical to SEQ ID NO: 220
  Synthetic aptamer

SEQ ID NO: 257 - DNA, 44 bp, Artificial Sequence
  Synthetic aptamer
  misc_feature: See specification as filed for detailed description of substitutions and preferred embodiments
  misc_feature (1)..(1): n is Nap-dU
  misc_feature (3)..(3): n is dA, dG or Nap-dU
  misc_feature (4)..(4): n is dA, dC or Nap-dU
  misc_feature (6)..(6): n is dA, dC or Nap-dU
  misc_feature (7)..(7): n is dC or Nap-dU
  misc_feature (9)..(9): n is dC or Nap-dU
  misc_feature (10)..(10): n is dA or Nap-dU
  misc_feature (12)..(12): n is dC or Nap-dU
  misc_feature (13)..(13): n is Nap-dU
  misc_feature (16)..(16): n is dC or Nap-dU
  misc_feature (20)..(20): n is Nap-dU
  misc_feature (22)..(22): n is Nap-dU
  misc_feature (24)..(24): n is Nap-dU
  misc_feature (27)..(27): n is dA, dG or Nap-dU
  misc_feature (28)..(28): n is Nap-dU
  misc_feature (29)..(29): n is Nap-dU
  misc_feature (33)..(33): n is dC or Nap-dU
  misc_feature (34)..(34): n is dC or Nap-dU
  misc_feature (37)..(37): n is Nap-dU
  misc_feature (39)..(39): n is dC or Nap-dU
  misc_feature (40)..(40): n is Nap-dU
  misc_feature (42)..(42): n is dC or Nap-dU
ncnncnncnnrnnarnrarnrncnrannnggannrrncnnmngg

SEQ ID NO: 258 - DNA, 35 bp, Artificial Sequence
  Synthetic aptamer
atctaatgaatgtctgccttgcactggtgcgttcc

SEQ ID NO: 259 - DNA, 35 bp, Artificial Sequence
  Synthetic aptamer
  misc_feature: See specification as filed for detailed description of substitutions and preferred embodiments
  misc_feature (1)..(1): n is dA, dC or Nap-dU
  misc_feature (2)..(2): n is dC or Nap-dU
  misc_feature (3)..(3): n is dC, dG or Nap-dU
  misc_feature (4)..(4): n is dA or Nap-dU
  misc_feature (5)..(5): n is dA, dG or Nap-dU
  misc_feature (6)..(6): n is dA or Nap-dU
  misc_feature (7)..(7): n is dA or Nap-dU
  misc_feature (11)..(11): n is Nap-dU
  misc_feature (13)..(13): n is Nap-dU
  misc_feature (15)..(15): n is Nap-dU
  misc_feature (17)..(17): n is dA, dC, dG or Nap-dU
  misc_feature (18)..(18): n is dC or Nap-dU
  misc_feature (19)..(19): n is Nap-dU
  misc_feature (20)..(20): n is Nap-dU
  misc_feature (24)..(24): n is dC or Nap-dU
  misc_feature (25)..(25): n is Nap-dU
  misc_feature (28)..(28): n is Nap-dU
  misc_feature (30)..(30): n is dC or Nap-dU
  misc_feature (31)..(31): n is dC, dG or Nap-dU
  misc_feature (32)..(32): n is dA, dG or Nap-dU
  misc_feature (33)..(33): n is dC or Nap-dU
  misc_feature (34)..(34): n is dA, dC, dG or Nap-dU
  misc_feature (35)..(35): n is dA, dC, dG or Nap-dU
nnnnnnnrarngncnvnnnngcannvgngnnnnnn

SEQ ID NO: 260 - DNA, 20 bp, Artificial Sequence
  Synthetic primer
ggtcgggcacactacgcatc

SEQ ID NO: 261 - DNA, 21 bp, Artificial Sequence
  Synthetic primer
gggaagagaaaggagaagaag

SEQ ID NO: 262 - DNA, 20 bp, Artificial Sequence
  Synthetic primer
ggtcgggcacactacgcatc

SEQ ID NO: 263 - DNA, 21 bp, Artificial Sequence
  Synthetic primer
gggaagagaaaggagaagaag

SEQ ID NO: 264 - protein, 13 aa, Artificial Sequence
  Synthetic amino acid
KCYGVSPTKLNDL

SEQ ID NO: 265 - protein, 11 aa, Artificial Sequence
  Synthetic amino acid
DSKVGGNYNYL

SEQ ID NO: 266 - protein, 17 aa, Artificial Sequence
  Synthetic amino acid
EIYQAGSTPCNGVEGFN

SEQ ID NO: 267 - protein, 18 aa, Artificial Sequence
  Synthetic amino acid
YGFQPTNGVGYQPYRVVV

SEQ ID NO: 268 - protein, 19 aa, Artificial Sequence
  Synthetic amino acid
FELLHAPATVCGPKKSTNL

(19) United States
(12) Patent Application Publication
(10) Pub. No.: US 2024/0218374 A1
(43) Pub. Date: Jul. 4, 2024

(54) Nucleic Acid Compounds That Bind Coronavirus Proteins

(71) Applicant: Somalogic Operating Co., Inc., Boulder, CO (US)

(72) Inventors: Amy D. Gelinas, Boulder, CO (US); Daniel J. Schneider, Arvada, CO (US); Nebojsa Janjic, Boulder, CO (US)

(73) Assignee: Somalogic Operating Co., Inc., Boulder, CO (US)

(21) Appl. No.: 18/285,743

(22) PCT Filed: Apr. 14, 2022
(86) PCT No.: PCT/US2022/024760

§ 371 Date: Oct. 5, 2023

Related U.S. Application Data
(60) Provisional application No. 63/174,747, filed on Apr. 14, 2021.

(51) Int. Cl. C12N 15/115; A61K 31/7115

(52) U.S. Cl. CPC C12N 15/115; A61K 31/7115; C12N 2310/16; C12N 2310/321; C12N 2310/334; C12N 2310/335; C12N 2320/13

(57) ABSTRACT

Described herein are aptamers capable of binding to coronavirus protein(s); compositions comprising a coronavirus protein binding aptamer with a coronavirus protein; and methods of making and using the same.

### CROSS-REFERENCE TO RELATED APPLICATIONS

This application claims the benefit of priority of U.S. Provisional Application No. 63/174,747, filed Apr. 14, 2021, which is incorporated by reference herein in its entirety for any purpose.

### FIELD

The present disclosure relates generally to the field of nucleic acids, and more specifically, to aptamers capable of binding to one or more SARS-CoV-2 protein, compositions comprising a SARS-CoV-2 protein binding aptamer and SARS-CoV-2 protein, methods of detecting SARS-CoV-2 proteins using such aptamers, and methods for treatment and prevention of SARS-CoV-2 infection using such aptamers.

### BACKGROUND

Since its first appearance in the human population at the end of 2019, Covid-19 has rapidly spread across the globe with a massive toll on human health with infection mortality rates of as high as 10% and a crippling impact on world economy. Despite numerous advances, there remains an urgent need for rapid, point-of-care diagnostic tests that do not require complex instrumentation, as well as for better therapeutic treatment options. To contribute chemically distinct, non-protein-based affinity reagents for this global effort, the present disclosure provides the identification of a DNA-based aptamers that target SARS-CoV-2 spike protein. These aptamer reagents target SARS-CoV-2 spike protein receptor binding domain and are capable of binding various constructs of the spike protein which include this domain (spike S1, spike S1 & S2 extracellular domain). The emergence of variant strains of SARS-CoV-2 have identified numerous point mutations within the spike protein. The DNA aptamer reagents identified here maintain high affinity binding to several of these mutant spike proteins as well as the more physiologically relevant spike trimer protein. Moreover, these DNA aptamer reagents inhibits the binding of the spike protein to its cell-surface receptor ACE2.

### SUMMARY

The present disclosure describes aptamers capable of binding to SARS-CoV-2 proteins. The aptamers are shown to be inhibitors of SARS-CoV-2 spike protein binding to the ACE2 receptor and these aptamers are further shown to reduce infectivity of SARS-CoV-2 and therefore can be useful as diagnostic and therapeutic agents. Pharmaceutical compositions comprising SARS-CoV-2 protein binding aptamers; and methods of making and using the same are described.

The following numbered paragraphs 1 through 56 contain statements of broad combinations of the inventive technical features herein disclosed:
1. An aptamer that binds a SARS-CoV-2 protein, wherein the aptamer comprises the sequence 5′-GDRATRXTAHRXRTXHTRAXHRXTXRRAXDDD-3′ (SEQ ID NO: 5) wherein,
A represents dA;
G represents dG;
C, T and X, each, independently, represent a C-5 modified pyrimidine nucleoside;
R is independently selected from a dA or dG;
H is independently selected from a dA, or a C-5 modified pyrimidine nucleoside; and
D is independently selected from a dA, dG or a C-5 modified pyrimidine nucleoside.
2. The aptamer of aspect 1, wherein C independently represents the C-5 modified pyrimidine Nap-dC.
3. The aptamer of aspect 1 or 2, wherein T independently represents the C-5 modified pyrimidine Tyr-dU.
4. The aptamer of any one of aspects 1 to 3, wherein X independently represents a C-5 modified pyrimidine selected from a Nap-dC or Tyr-dU.
5. The aptamer of aspect 4, wherein X independently represents the C-5 modified pyrimidine Nap-dC.
6. The aptamer of aspect 4, wherein X independently represents the C-5 modified pyrimidine Tyr-dU
7. The aptamer of any one of aspects 1 to 6, wherein H independently represents a C-5 modified pyrimidine selected from a Nap-dC or Tyr-dU.
8. The aptamer of aspect 7, wherein H independently represents the C-5 modified pyrimidine Nap-dC.
9. The aptamer of aspect 7, wherein H independently represents the C-5 modified pyrimidine Tyr-dU.
10. The aptamer of any one of aspects 1 to 9, wherein D independently represents the C-5 modified pyrimidine Tyr-dU.
11. The aptamer of aspect 1, wherein the aptamer comprises the sequence 5′-GGGATACTATGCGTCCGACCGCTCGGACGGA-3′ (SEQ ID NO: 4) wherein,
A represents dA;
G represents dG;
C represents Nap-dC; and
T represents Tyr-dU.
12. The aptamer of aspect 1, wherein the aptamer comprises a sequence selected from SEQ ID NOs: 4, 6-20 and 28-122.
13. A heterodimeric aptamer that binds a SARS-CoV-2 protein, wherein the heterodimeric aptamer comprises a first aptamer comprising the sequence 5′-G G G A Y A p Y A Y G p G Y p p G A p p G p Y p G G A p G-3′ (SEQ ID NO: 7), a linkage covalently bonding the first aptamer to a second aptamer, and a second aptamer which binds a SARS-CoV-2 protein at a nonoverlapping binding site relative to the binding site of the first aptamer, wherein,
Y and p, each, independently, represent a C-5 modified pyrimidine nucleoside.
14. The heterodimeric aptamer of aspect 13, wherein Y independently represents the C-5 modified pyrimidine 5-(p-hydroxyphenethyl)-1-aminocarbonyl-2′-deoxyuridine.
15. The heterodimeric aptamer of aspect 13, wherein p independently represents the C-5 modified pyrimidine 5-(1-Naphthylmethyl)aminocarbonyl-2′-deoxycytidine.
16. The heterodimeric aptamer of any one of aspects 13-15, wherein the first aptamer is linked 5′ of the second aptamer.
17. The heterodimeric aptamer of any one of aspects 13-15, wherein the first aptamer is linked 3′ of the second aptamer.
18. The heterodimeric aptamer of any of aspects 13-17, wherein the linkage is a hexaethylene glycol (HEG) linkage.
19. The aptamer of any one of aspects 1-12, wherein the aptamer is a first aptamer, further wherein the first aptamer is covalently linked to a second aptamer that binds a SARS-CoV-2 protein at a nonoverlapping binding site relative to the binding site of the first aptamer.
20. The aptamer of aspect 19, wherein the first aptamer comprises a sequence selected from SEQ ID NOs: 4, 6-20 and 28-122.
21. The aptamer of aspect 20, wherein the first aptamer comprises the nucleotide sequence of SEQ ID NO:4.
22. The aptamer of any one of aspects 19-21, wherein the first aptamer is linked 5′ of the second aptamer.
23. The aptamer of any one of aspects 19-21, wherein the first aptamer is linked 3′ of the second aptamer.
24. The aptamer of any one of aspects 19 to 23, wherein the linkage is a hexaethylene glycol (HEG) linkage.
25. The aptamer of any one of aspects 1 to 24, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.
26. The aptamer of any one of aspects 1 to 25, wherein the SARS-CoV-2 protein comprises an amino acid sequence selected from any one of SEQ ID NOs: 21-27.
27. An aptamer that binds a SARS-CoV-2 protein, wherein the aptamer comprises a sequence that is at least 80%, at least 85%, at least 90%, at least 95%, at least 97%, at least 99%, or 100% identical to a sequence selected from SEQ ID NOs: 4, 6-20 and 28-122.
28. The aptamer of any one of the preceding aspects, wherein the aptamer has a dissociation constant (K_{d}) for the SARS-CoV-2 protein of 2 pM to 10 nM.
29. The aptamer of any one of the preceding aspects, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
30. The aptamer of any one of the preceding aspects, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
31. The aptamer of any one of the preceding aspects, comprising at least one C-5 modified pyrimidine which is 5-(N-naphthylmethylcarboxyamide)-2′-deoxyuridine (NapdU).
32. The aptamer of any one of the preceding aspects, wherein the aptamer comprises at least one 2′-O-methyl modified nucleotide.
33. The aptamer of any one of the preceding aspects, wherein the aptamer is 24 to 100 nucleotides in length, or 30 to 60 nucleotides in length, or 28 to 60 nucleotides in length, or 28 to 50 nucleotides in length, or 28 to 40 nucleotides in length, or 40 to 50 nucleotides in length, or 28 to 32 nucleotides in length.
34. The aptamer of any one of the preceding aspects, wherein the SARS-CoV-2 protein is the SARS-CoV-2 spike receptor binding domain (RBD).
35. The aptamer of aspect 34, wherein the aptamer inhibits binding of the SARS-CoV-2 RBD to an angiotensin-converting enzyme 2 (ACE2) receptor.
36. The aptamer of any one of the preceding aspects, wherein the aptamer inhibits SARS-CoV-2 viral cell membrane fusion with a host cell.
37. The aptamer of any one of the preceding aspects, wherein the aptamer inhibits SARS-CoV-2 viral infection of human cells.
38. The aptamer of aspect 37, wherein the IC₅₀ of the aptamer is less than 2.0E-9 (M).
39. A composition comprising the aptamer of any one of the preceding aspects and a SARS-CoV-2 protein.
40. A pharmaceutical composition comprising a therapeutically effective amount of the aptamer of any one of aspects 1 to 38 and at least one pharmaceutically acceptable excipient.
41. A method of treating or preventing a SARS-CoV-2 infection in a human, comprising administering a therapeutically effective amount of the aptamer of any one of aspects 1 to 38 or the pharmaceutical composition of aspect 40 to the human.
42. A method for detecting the presence of SARS-CoV-2 in a sample, comprising contacting the sample with the aptamer of any one of aspects 1 to 38.
43. The method of aspect 42, wherein the sample is in vitro.
44. A method for selecting an aptamer having binding affinity for a SARS-CoV-2 protein comprising: (a) contacting a candidate mixture with a SARS-CoV-2 protein, wherein the candidate mixture comprises modified nucleic acids in which one, several or all pyrimidines in at least one, or each, nucleic acid of the candidate mixture comprises a C-5 modified pyrimidine nucleoside; (b) exposing the candidate mixture to a slow off-rate enrichment process, wherein nucleic acids having a slow rate of dissociation from the target molecule relative to other nucleic acids in the candidate mixture bind the SARS-CoV-2 protein, forming nucleic acid-target molecule complexes; (c) partitioning slow off-rate nucleic acids from the candidate mixture; (d) amplifying the slow off-rate nucleic acids to yield a mixture of nucleic acids enriched in nucleic acid sequences that are capable of binding to the SARS-CoV-2 protein with a slow off-rate, whereby a slow off-rate aptamer to the SARS-CoV-2 protein molecule is selected.
45. The method of aspect 44, wherein each nucleic acid is, independently, from 24 to 100 nucleotides in length, or from 30 to 60 nucleotides in length, or from 28 to 60 nucleotides in length, or from 40 to 50 nucleotides in length, or 28 nucleotides in length.
46. The method of aspect 44 or 45, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
47. The method of any one of aspects 44 to 46, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
48. The method of any one of aspects 44 to 47, wherein at least one C-5 modified pyrimidine is 5-(N-naphthylmethylcarboxyamide)-2′-deoxyuridine (NapdU).
49. The method of any one of aspects 44 to 48, wherein a plurality of nucleic acids in the mixture comprise at least one 2′-O-methyl modified nucleotide.
50. The method of any one of aspects 44 to 49, wherein a plurality of nucleic acids in the mixture comprise a linker selected from a 3-carbon-spacer, a hexaethylene glycol linker, a polyethylene glycol linker or any combination thereof.
51. The method of any one of aspects 44 to 50, wherein the SARS-CoV-2 protein is selected from SARS-CoV-2 spike receptor binding domain (RBD), SARS-CoV-2 spike 51, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.
52. A method for inhibiting binding of a SARS-CoV-2 protein to an angiotensin-converting enzyme 2 (ACE2) receptor, comprising contacting the SARS-CoV-2 protein with the aptamer of any one of aspects 1-38.
53. The method of aspect 52, wherein the SARS-CoV-2 protein is in a sample in vitro.
54. The method of aspect 52, wherein the SARS-CoV-2 protein is in a human.
55. The method of any one of aspects 52 to 54, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.
56. The method of any one of aspects 52 to 55, wherein the SARS-CoV-2 protein is the SARS-CoV-2 RBD.
57. An aptamer that binds a SARS-CoV-2 protein, wherein the aptamer comprises the sequence 5′-DRHRRXWXWTGRXWXXTXDWDTXRARHR-3′ (SEQ ID NO: 253) or 5′-TRXDRXRXWXXWTWTTHRRXHTRRRNDB-3′ (SEQ ID NO: 255)
   wherein
A is dA;
G is dG;
each C is independently, and for each occurrence, is a C-5 modified pyrimidine nucleoside;
each T is independently, and for each occurrence, is a C-5 modified pyrimidine nucleoside;
each R is independently, and for each occurrence, is dA or dG; each W is independently, and for each occurrence, is dA or a C-5 modified pyrimidine nucleoside;
each X is independently, and for each occurrence, is a C-5 modified pyrimidine nucleoside;
each H is independently, and for each occurrence, is dA or a C-5 modified pyrimidine nucleoside;
each D is independently, and for each occurrence, is dA, dG or C-5 modified pyrimidine nucleoside;
each B is independently, and for each occurrence, is dG or a C-5 modified pyrimidine dU; and each N is independently, and for each occurrence, is dA, dG or a C-5 modified pyrimidine nucleoside.
58. The aptamer of aspect 57, wherein C is the C-5 modified pyrimidine Nap-dC.
59. The aptamer of aspect 57 or 58, wherein T is the C-5 modified pyrimidine Tyr-dU.
60. The aptamer of any one of aspects 57 to 59, wherein W is dA or the C-5 modified pyrimidine Tyr-dU.
61. The aptamer of any one of aspects 57 to 60, wherein X is the C-5 modified pyrimidine Nap-dC or Tyr-dU.
62. The aptamer of any one of aspects 57 to 61, wherein H is dA or the C-5 modified pyrimidine Nap-dC or Tyr-dU
63. The aptamer of any one of aspects 57 to 62, wherein D is dA, dG or the C-5 modified pyrimidine Tyr-dU.
64. The aptamer of any one of aspects 57 to 63, wherein B is dG or the C-5 modified pyrimidine Nap-dC or Tyr-dU.
65. The aptamer of any one of aspects 57 to 64, wherein N is dA, dG or the C-5 modified pyrimidine Nap-dC or Tyr-dU.
66. The aptamer of aspect 57 wherein the aptamer comprises the sequence 5′-GGCGGCACATGGCACTTCATATCGAGCG-3′ (SEQ ID NO: 252) or the sequence 5′-TGCAACGCACCTTATTCGGCTTGAATGT-3′ (SEQ ID NO: 254) wherein,
A represents dA;
G represents dG;
C represents Nap-dC; and
T represents Tyr-dU.
67. An aptamer that binds a SARS-CoV-2 protein, wherein the aptamer comprises the sequence 5′-TCDHCHXCXWRXTARXRARTRTCTRADTTGGAXXRRTCXTMXGG-3′ (SEQ ID NO: 257) or 5′-HXBWDWWRARTGTCTVNXTTGCAXTVGTGXBDXNN (SEQ ID NO: 259)-3′, wherein
A is dA;
G is dG;
C is dC;
each T is independently, and for each occurrence, is a C-5 modified pyrimidine nucleoside;
each R is independently, and for each occurrence is dA or dG;
each M is independently, and for each occurrence is dA or dC;
each W is independently, and for each occurrence is dA or a C-5 modified pyrimidine nucleoside;
X is independently, and for each occurrence is dC or a C-5 modified pyrimidine nucleoside;
H is independently, and for each occurrence is dA, dC or a C-5 modified pyrimidine nucleoside;
D is independently, and for each occurrence is dA, dG or a C-5 modified pyrimidine nucleoside;
V is independently, and for each occurrence is dA, dC or dG;
B is independently, and for each occurrence is dC, dG or a C-5 modified pyrimidine nucleoside; and
N is independently, and for each occurrence is dA, dC, dG or a C-5 modified pyrimidine nucleoside.
68. The aptamer of aspect 67, wherein T is the C-5 modified pyrimidine NapdU.
69. The aptamer of aspect 67 or 68, wherein R is dA or dG.
70. The aptamer of any one of aspects 67 to 69, wherein M is dA or dC.
71. The aptamer of any one of aspects 67 to 70, where W is dA or the C-5 modified pyrimidine Nap-dU.
72. The aptamer of any one of aspects 67 to 71, wherein X is dC or the C-5 modified pyrimidine Nap-dU.
73. The aptamer of any one of aspects 67 to 72, wherein H is dA, dC or the C-5 modified pyrimidine Nap-dU.
74. The aptamer of any one of aspects 67 to 73, wherein D is dA, dG or the C-5 modified pyrimidine Nap-dU.
75. The aptamer of any one of aspects 67 to 74, wherein V is dA, dC or dG.
76. The aptamer of any one of aspects 67 to 75, wherein B is dC, dG or the C-5 modified pyrimidine Nap-dU.
77. The aptamer of any one of aspects 67 to 76, wherein N is dA, dC, dG or the C-5 modified pyrimidine Nap-dU.
78. An aptamer that binds a SARS-CoV-2 protein, wherein the aptamer comprises a sequence that is at least 80%, at least 85%, at least 90%, at least 95%, at least 97%, at least 99%, or 100% identical to a sequence selected from SEQ ID NOs: 128-252, 254, 256 and 258.
79. The aptamer of any one of aspects, 57 to 78, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
80. The aptamer of any one of aspects 57 to 78, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).
81. The aptamer of any one of aspects 57 to 80, wherein the SARS-CoV-2 protein is selected from SARS-CoV-2 spike receptor binding domain (RBD), SARS-CoV-2 spike S1, spike S1 & S2 extracellular domain (ECD), and spike S1 & S2 ECD stable trimer.
82. The aptamer of any one of the preceding aspects 57 to 81, wherein the aptamer inhibits binding of the SARS-CoV-2 RBD to an angiotensin-converting enzyme 2 (ACE2) receptor.
83. The aptamer of any one of the preceding aspects 57 to 82, wherein the aptamer inhibits SARS-CoV-2 viral cell membrane fusion with a host cell.
84. The aptamer of any one of the preceding aspects 57 to 83, wherein the aptamer inhibits SARS-CoV-2 viral infection of human cells.
85. A composition comprising the aptamer of any one of the preceding aspects 57 to 84.
86. A pharmaceutical composition comprising a therapeutically effective amount of the aptamer of any one of claims 57 to 84 and at least one pharmaceutically acceptable excipient.
87. A method of treating or preventing a SARS-CoV-2 infection in a subject, comprising administering a therapeutically effective amount of the aptamer of any one of claims 57 to 84 or the pharmaceutical composition of claim 86 to the subject.
88. A method for detecting the presence of SARS-CoV-2 in a sample, comprising contacting the sample with the aptamer of any one of claims 57 to 84.
89. The method of aspect 88, wherein the sample is in vitro.
90. The method of any one of aspects 87 to 89, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K),), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.
91. A method for inhibiting binding of a SARS-CoV-2 protein to an angiotensin-converting enzyme 2 (ACE2) receptor, comprising contacting the SARS-CoV-2 protein with the aptamer of any one of claims 57 to 84.
92. The method of aspect 91, wherein the SARS-CoV-2 protein is in a sample in vitro.
93. The method of aspect 91, wherein the SARS-CoV-2 protein is in vivo.

### BRIEF DESCRIPTION OF THE DRAWINGS

FIG. 1 shows the consensus sequence of aptamer 26874-29_4 (SEQ ID NO: 5). Row (A) indicates the frequency at which dA is observed in the C1 aptamer family at each of the 31 conserved positions. Row (C) indicates the frequency at which Nap-dC is observed in the C1 aptamer family at each of the 31 conserved positions. Row (G) indicates the frequency at which dG is observed in the C1 aptamer family at each of the 31 conserved positions. Row (T) indicates the frequency at which Tyr-dU is observed in the C1 aptamer family at each of the 31 conserved positions. Row (preferred) indicates the highest frequency nucleotide at each of the 31 conserved positions. Row (Consensus) indicates the consensus sequence for the aptamer, where: A=dA; G=dG; C=Nap-dC; T=Tyr-dU; R=dA or dG; X=Nap-dC or Tyr-dU; H=dA, Nap-dC or Tyr-dU; and D=dA, dG or Tyr-dU.

FIGS. 2A-2H illustrate dose-dependent binding of aptamer 26874-29_4 and aptamer 26874-29_20 to various forms of the SARS-CoV-2 spike protein. For each binding curve, the fraction of aptamer-protein complex was plotted as a function of spike protein concentration. Equilibrium binding constants (K_{d} values) were determined by fitting the data to a four-parameter sigmoid dose-response model. FIG. 2A shows 26874-29_4 binding SARS-CoV-2 spike receptor binding domain (RBD); FIG. 2B shows 26874-29_4 binding SARS-CoV-2 spike S1; FIG. 2C shows 26874-29_4 binding SARS-CoV-2 spike S1 & S2 extracellular domain (ECD); FIG. 2D shows 26874-29_20 binding SARS-CoV-2 spike S1 & S2 ECD; FIG. 2E shows 26874-29_20 binding SARS-CoV-2 spike S1 & S2 ECD stable trimer; FIG. 2F shows 26874-29_20 binding SARS-CoV-2 spike S1 aspartic acid 614 to glycine mutant (D614G); FIG. 2G shows 26874-29_4 binding spike RBD asparagine 501 to tyrosine mutant (N501Y); FIG. 2H shows 26874-29_20 binding spike RBD glutamic acid 484 to lysine mutant (E484K).

FIG. 3 illustrates dose-dependent inhibition of the spike RBD-ACE2 interaction by aptamer 26874-29_4 in a 96-well plate-based assay. Percent spike RBD activity was plotted as a function of 26874-29_4 concentration relative to no aptamer control.

FIGS. 4A-4B illustrate dose-dependent neutralization of authentic SARS-CoV-2 virus by 26874-29_4 (FIG. 4A) and 26874-29_20 (FIG. 4B) in a foci reduction neutralization assay. The percentage of foci forming units with the test aptamers relative to control was plotted as a function of aptamer concentration. The IC50 values were determined by fitting the data to a variable slope dose vs response curve. The calculated IC50 values from triplicate measurements for 26874-29_4 and 26874-29_20 are 1.2 nM and 1.1 nM, respectively.

FIGS. 5A-5C illustrate exemplary 5-position modified uracils and cytosines that may be incorporated into aptamers.

FIGS. 6A-6B illustrate exemplary modifications that may be present at the 5-position of uracil. The chemical structure of the C-5 modification includes the exemplary amide linkage that links the modification to the 5-position of the uracil. The 5-position moieties shown include a benzyl moiety (e.g., Bn, PE, and a PP), a naphthyl moiety (e.g., Nap, 2Nap, NE, 2NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), an indole moiety (e.g, Trp) and a hydroxypropyl moiety (e.g., Thr).

FIGS. 7A-7B illustrate exemplary C-5 modified pyrimidine containing nucleosides and modifications that may be present at the 5-position of cytosine. The chemical structure of the C-5 modification includes the exemplary amide linkage that links the modification to the 5-position of the cytosine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

FIGS. 8A-8B illustrate heterodimer constructs of aptamer 26874-29_20 covalently linked to a second aptamer sequence through a flexible hexaethylene glycol linker (HEG). (FIG. 8A) Aptamer 26874-29_20 is located on the 5′ end of the heterodimer with a second aptamer sequence on the 3′ end. (FIG. 8B) Aptamer 26874-29_20 is located on the 3′ end of a heterodimer with a second aptamer sequence on the 5′ end.

FIG. 9 shows the consensus sequence of aptamer 26876-3_18 (SEQ ID NO: 235). Row (A) indicates the frequency at which dA is observed in the aptamer family at each of the 28 conserved positions. Row (C) indicates the frequency at which Nap-dC is observed in the aptamer family at each of the 28 conserved positions. Row (G) indicates the frequency at which dG is observed in the aptamer family at each of the 28 conserved positions. Row (T) indicates the frequency at which Tyr-dU is observed in the aptamer family at each of the 28 conserved positions. Row (preferred) indicates the highest frequency nucleotide at each of the 28 conserved positions. Row (Consensus) indicates the consensus sequence for the aptamer, where: A=dA; G=dG; C=Nap-dC; T=Tyr-dU; R=dA or dG; W=dA or Tyr-dU; X=Nap-dC or Tyr-dU; H=dA, Nap-dC or Tyr-dU; and D=dA, dG or Tyr-dU.

FIG. 10 shows the consensus sequence of aptamer 26876-13_4 (SEQ ID NO: 239). Row (A) indicates the frequency at which dA is observed in the aptamer family at each of the 28 conserved positions. Row (C) indicates the frequency at which Nap-dC is observed in the aptamer family at each of the 28 conserved positions. Row (G) indicates the frequency at which dG is observed in the aptamer family at each of the 28 conserved positions. Row (T) indicates the frequency at which Tyr-dU is observed in the aptamer family at each of the 28 conserved positions. Row (preferred) indicates the highest frequency nucleotide at each of the 28 conserved positions. Row (Consensus) indicates the consensus sequence for the aptamer, where: A=dA; G=dG; C=Nap-dC; T=Tyr-dU; R=dA or dG; W=dA or Tyr-dU; X=Nap-dC or Tyr-dU; H=dA, Nap-dC or Tyr-dU; D=dA, dG or Tyr-dU; B=Nap-dC, dG or Tyr-dU; and N=dA, Nap-dC, dG or Tyr-dU.

FIG. 11 shows the consensus sequence of aptamer 26860-75_3 (SEQ ID NO: 207). Row (A) indicates the frequency at which dA is observed in the aptamer family at each of the 44 conserved positions. Row (C) indicates the frequency at which dC is observed in the aptamer family at each of the 44 conserved positions. Row (G) indicates the frequency at which dG is observed in the aptamer family at each of the 44 conserved positions. Row (T) indicates the frequency at which Nap-dU is observed in the aptamer family at each of the 44 conserved positions. Row (preferred) indicates the highest frequency nucleotide at each of the 44 conserved positions. Row (Consensus) indicates the consensus sequence for the aptamer, where: A=dA; G=dG; C=dC; T=Nap-dU; R=dA or dG; M=dA or dC; W=dA or Nap-dU; X=dC or Nap-dU; H=dA, dC or Nap-dU; and D=dA, dG or Nap-dU.

FIG. 12 shows the consensus sequence of aptamer 26860-16_18 (SEQ ID NO: 203). Row (A) indicates the frequency at which dA is observed in the aptamer family at each of the 35 conserved positions. Row (C) indicates the frequency at which dC is observed in the aptamer family at each of the 35 conserved positions. Row (G) indicates the frequency at which dG is observed in the aptamer family at each of the 35 conserved positions. Row (T) indicates the frequency at which Nap-dU is observed in the aptamer family at each of the 35 conserved positions. Row (preferred) indicates the highest frequency nucleotide at each of the 35 conserved positions. Row (Consensus) indicates the consensus sequence for the aptamer, where: A=dA; G=dG; C=dC; T=Nap-dU; R=dA or dG; M=dA or dC; W=dA or Nap-dU; X=dC or Nap-dU; H=dA, dC or Nap-dU; D=dA, dG or Nap-dU; V=dA, dC or dG; B=dC, dG or Nap-dU; and N=dA, dC, dG or Nap-dU.

FIG. 13A-13E shows affinities of partially truncated select SOMAmer reagents arising from selections with five spike protein variants. SOMAmers were screened for binding their respective SELEX targets; spike S1/S2 ECD (13A), spike S1 domain (13B), spike S2 domain (13C), spike receptor binding domain (13D), and the spike S1 nCoV-1/nCoV-2 toggle (13E), either as 50-mers for single modifications (40N random region plus five nucleotides from both the 5′ and 3′ primer regions), or as 40-mers for dual-modified sequences (30N random region plus five nucleotides from both the 5′ and 3′ primer regions). Equilibrium binding dissociation constants (K_{d}) were measured in a filter binding assay with radiolabeled SOMAmer. Line in box plot represent the median of K_{d} values.

FIG. 14 shows inhibition of binding of the spike protein to ACE2 receptor by select SOMAmers and antibodies. SOMAmer reagents show a dose-dependent inhibition of spike-ACE2 over a concentration range of 10⁻⁷ to 10⁻¹² M with half-maximal inhibitory concentrations (IC50) in the same range as commercially available spike antibodies. Percent activity was determined by calculating the fraction of the positive control signal (ACE2 plus spike RBD). Data were fit to a four-parameter dose response curve.

FIG. 15A-15B show SOMAmer reagents in a foci neutralization assay. (15A) Normalized infectivity data in the presence of 92 SOMAmer reagents tested at a single concentration of 10 nM. Thirteen reagents having greater than 20% reduction in plaque count (within the green shading) were subject to a full titration. Titration of the thirteen reagents in the foci neutralization assay showed a range of effects with SL26876-13_4 (15B) having no neutralizing effect, SL26874-29_4 (15C) being the most potent, and SL26845-63_3 (15D) having a biphasic profile. Assay repeated three times in triplicate and data were plotted in GraphPad Prism and fit to either a biphasic curve or a four-parameter dose-response curve.

FIG. 16A-16J show titration of SOMAmer reagents in foci reduction assay showed a range of effects. (16A) SL1110 and (16B) SL1107 are biphasic, having both high and low potency components. (16C) SL1109, (16D) SL1106, (16E) SL1105, (16F) SL1112, (16G) SL1113, and (16H) SL1108 have a neutralizing effect and inhibition curves with Hill slopes of −0.7 to −1. (16I) SL1114 and (16J) SL1116 have no neutralization effect. SOMAmer reagents in panels (16A) through (16H) were either selected to or have binding affinity for spike RBD. SOMAmers in panels (16I) and (16J) were selected to spike S2. Assay was repeated three times in triplicate and data were plotted in GraphPad Prism and fit to either a biphasic curve or a four-parameter dose-response curve.

FIG. 17A-17D show the results of a foci-reduction neutralization assay for 26860-75_3 (17A), 26860-16_3 (17B), 26876-13_4 (17C), 26876-3_4 (17D).

FIG. 18A-18B show competition of neutralization assay leads with radiolabeled SL1111 ligand and unlabeled competitors. (18A) SOMAmer competitors directly compete with SL1111 for binding spike S1/S2 protein. (18B) Six SOMAmer leads do not displace SL1111 when binding spike S1/S2 protein. Data were fit to a one-site competition model of competitor concentration vs fraction no competitor.

FIG. 19A-19D show SL1111_20 Epitope Mapping with Structurally Characterized Antibodies. (19A). Competition Elisa results show that SL1111_20 partially blocks binding to spike RBD for a subset of class 3 and 4 antibodies but does not compete with any class 1 or 2 antibodies. Data presented as mean±SEM. (19B) Class 3 and 4 antibody epitopes on spike RBD affected by prior equilibration of RBD with SL111_20. (19C) Class 3 and 4 antibody epitopes on spike RBD that are non-competitive with SL1111_20. (19D) Representation of class 1 and 2 antibody epitopes not competitive with SL1111_20. Epitopes were determined by defining residues on spike RBD within 4 Å of the antibodies. Residues were then mapped onto the spike RBD/ACE2 crystal structure, PDB 6M0J. PDB structures used for determining RBD epitopes: VHH72, 6WAQ; CR3022, 6YLA; REGN10987, 6XDG; EY6A, 6ZER; S309, 7R6W; cc121, 6XC2; REGN10933, 6XDG.

FIG. 20A-20B shows potential contact residues of SL1111_20 on spike RBD. (20A) Amino acid sequence of spike RBD protein (SE ID NO: 21) with mutations from the VOCs indicated below. Potential contact residues of SL1111_20 determined from known crystal structures of antibodies bound to spike RBD in combination with the competition Elisa data is shown above. Potential contact residues of SL1111_20 were determined by identifying unique residues on spike RBD found only in antibodies blocked by SL1111_20. X indicates deletion. (20B) Contact sites of mABs inhibited by SL1111.

FIG. 21A-21C show serum stability of SARS-CoV-2 spike reagents and unmodified analogs. (21A) Modified lead SOMAmers and their corresponding dT and dT/dC analogs were incubated in 90% pooled human serum and sample was removed at various time points (3 hours to 96 hours) and analyzed via denaturing PAGE. Sample timepoints from (21A) were plotted as the percent intact reagent and fit to a one phase exponential decay model to determine half life for duel-modified sequences and their dT/dC analogs (21B), and single-modified sequences and their dT analogs (21C).

FIG. 22 shows SL1111_20 Potently neutralizes prototype authentic SARS-CoV-2 (B Victoria, circle), Delta (triangle), Gamma (inverted triangle) and Omicron (square) VOCs. SL1111_20 shows enhanced neutralization activity against Omicron, Gamma and Delta variants compared to the prototype Clade B, VIC01.

FIG. 23 shows K_{d} values and affinity ratios for truncated SOMAmer reagents binding recombinant wt spike S1/S2 monomeric protein, spike S1/S2 stable trimer protein and spike S1/S2 trimer variants.

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aptamer: The term aptamer, as used herein, refers to a non-naturally occurring nucleic acid that has a desirable action on a target molecule. Desirable actions include, but are not limited to, binding of the target, enhancing the activity of the target, and inhibiting the activity of the target. An aptamer may also be referred to as a “nucleic acid ligand.” In some embodiments, an aptamer is a SOMAmer. As used herein, the term “aptamer” includes aptamers and pharmaceutically acceptable salts thereof, unless specifically indicated otherwise.

In some embodiments, an aptamer specifically binds spike through a mechanism which is independent of Watson/Crick base pairing or triple helix formation, and wherein the aptamer does not have the known physiological function of being bound by spike. In some embodiments, aptamers that bind spike include nucleic acids that are identified from a candidate mixture of nucleic acids, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers that bind spike are identified. It is recognized that affinity interactions are a matter of degree; however, in this context, an aptamer that “specifically binds” its target means that the aptamer binds to its target with a much higher degree of affinity than it binds to other, non-target, components in a mixture or sample. An “aptamer” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may comprise DNA, RNA, both DNA and RNA, and modified versions of either or both, and may be single stranded, double stranded, or contain double stranded or triple stranded regions, or any other three-dimensional structures.

Bioactivity: The term bioactivity, as used herein, refers to one or more intercellular, intracellular or extracellular process (e.g., cell-cell binding, ligand-receptor binding, cell signaling, etc.) which can impact physiological or pathophysiological processes.

C-5 Modified Pyrimidine: C-5 modified pyrimidine, as used herein, refers to a pyrimidine with a modification at the C-5 position. Examples of a C-5 modified pyrimidine include those described in U.S. Pat. Nos. 5,719,273 and 5,945,527. Certain nonlimiting examples of C-5 modified pyrimidines are provided herein.

Spike Aptamer: “spike aptamer”, as used herein, refers to an aptamer that is capable of binding to a spike protein.

Modified: As used herein, the terms “modify”, “modified”, “modification”, and any variations thereof, when used in reference to an oligonucleotide, means that the oligonucleotide comprises at least one non-natural moiety, such as at least one non-natural sugar moiety, at least one non-natural internucleoside linkage, at least one non-natural nucleotide base moiety, and/or at least one moiety that does not naturally occur in oligonucleotides (such as, for example, a 3 carbon spacer or a hexaethylene glycol (HEG)). In some embodiments, at least one of the four constituent nucleotide bases (i.e., A, G, T/U, and C) of the oligonucleotide is a modified nucleotide. In some such embodiments, the modified nucleotide comprises a base moiety that is more hydrophobic than the naturally-occurring base. In some embodiments, the modified nucleotide confers nuclease resistance to the oligonucleotide. In some embodiments, when an aptamer comprises one or more modified nucleotides that comprise hydrophobic base moieties, the aptamer binds to its target, such as a protein, through predominantly hydrophobic interactions. In some embodiments, such hydrophobic interactions result in high binding efficiency and stable co-crystal complexes. A pyrimidine with a substitution at the C-5 position is an example of a modified nucleotide. Modifications can also include 3′ and 5′ modifications, such as capping. Other modifications can include substitution of one or more of the naturally occurring nucleotides with an analog, internucleoside modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers, in some embodiments, ranging from about 10 to about 80 kDa, PEG polymers, in some embodiments, ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Modulate: As used herein, “modulate” means to alter, either by increasing or decreasing, the level, stability, processing, and/or activity of a target.

Nucleic Acid: As used herein, “nucleic acid,” “oligonucleotide,” and “polynucleotide” are used interchangeably to refer to a polymer of nucleotides and include DNA, RNA, DNA/RNA hybrids and modified versions of such entities. The terms “polynucleotide,” “oligonucleotide,” and “nucleic acid” include double- or single-stranded molecules as well as triple-helical molecules. The term nucleic acid includes aptamers, but is not limited thereto (i.e., the term includes other polymers of nucleotides).

Nuclease: As used herein, the term “nuclease” refers to an enzyme capable of cleaving the phosphodiester bond between nucleotide subunits of an oligonucleotide. As used herein, the term “endonuclease” refers to an enzyme that cleaves phosphodiester bond(s) at a site internal to the oligonucleotide. As used herein, the term “exonuclease” refers to an enzyme which cleaves phosphodiester bond(s) linking the end nucleotides of an oligonucleotide. Biological fluids typically contain a mixture of both endonucleases and exonucleases.

Nuclease Resistant: As used herein, the terms “nuclease resistant” and “nuclease resistance” refer to the reduced ability of an oligonucleotide to serve as a substrate for an endo- or exonuclease, such that, when contacted with such an enzyme, the oligonucleotide is either not degraded or is degraded more slowly or to a lesser extent than a control oligonucleotide of similar length and sequence but lacking one or more modifications of the oligonucleotide whose nuclease resistance is being measured.

Nucleotide: As used herein, the term “nucleotide” refers to a ribonucleotide or a deoxyribonucleotide, or a modified form thereof. Nucleotides include species that include purines (e.g., adenine, hypoxanthine, guanine, and the like) as well as pyrimidines (e.g., cytosine, uracil, thymine, and the like). When a base is indicated as “A”, “C”, “G”, “U”, or “T”, it is intended to encompass both ribonucleotides and deoxyribonucleotides, and modified forms thereof.

Pharmaceutically Acceptable: Pharmaceutically acceptable, as used herein, means approved by a regulatory agency of a federal or a state government or listed in the U.S.

Pharmacopoeia or other generally recognized pharmacopoeia for use in animals and, more particularly, in humans.

Pharmaceutically Acceptable Salt: Pharmaceutically acceptable salt of a compound (e.g., aptamer), as used herein, refers to a product that contains the compound and one or more additional pharmaceutically-acceptable atoms or groups bound to the compound through ionic bond(s). In some embodiments, a pharmaceutically acceptable salt is produced by contacting the compound with an acid or a base. A pharmaceutically acceptable salt may include, but is not limited to, acid addition salts including hydrochlorides, hydrobromides, phosphates, sulphates, hydrogen sulphates, alkylsulphonates, arylsulphonates, arylalkylsulfonates, acetates, benzoates, citrates, maleates, fumarates, succinates, lactates, and tartrates; alkali metal cations such as Li, Na, K, alkali earth metal salts such as Mg or Ca, or organic amine salts.

Pharmaceutical Composition: Pharmaceutical composition, as used herein, refers to a formulation comprising a compound (such as an aptamer) in a form suitable for administration to an individual. A pharmaceutical composition is typically formulated to be compatible with its intended route of administration. Examples of routes of administration include, but are not limited to, intravitreal, enteral and parenteral, including, e.g., subcutaneous injection or infusion, intravenous injection or infusion, intra-articular injection, intra-artery injection and infusion, intra-aqueous humor injection and implantation, and intra-vitreous injection and implantation.

Protein: As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the purified protein is obtained, or substantially free from chemical precursors or other chemicals when chemically synthesized.

The terms “SARS-CoV-2 spike”, “SARS-CoV-2 spike protein”, “spike protein” and “spike” may be used interchangeably throughout and have the same meaning.

SELEX: The term SELEX, as used herein, refers to generally to the selection for nucleic acids that interact with a target molecule in a desirable manner, for example binding with high affinity to a protein; and the amplification of those selected nucleic acids. SELEX may be used to identify aptamers with high affinity to a specific target molecule. The term SELEX and “SELEX process” may be used interchangeably. In some embodiments, methods of selecting aptamers that bind to spike are provided, comprising: (a) preparing a candidate mixture of nucleic acids, wherein the candidate mixture comprises modified nucleic acids in which at least one pyrimidine in at least one, or in each, nucleic acid of the candidate mixture is chemically modified at the C5-position; (b) contacting the candidate mixture with spike, wherein nucleic acids having an increased affinity to spike relative to other nucleic acids in the candidate mixture bind spike, forming nucleic acid-spike complexes; (c) partitioning the increased affinity nucleic acids from the remainder of the candidate mixture; and (d) amplifying the increased affinity nucleic acids to yield a mixture of nucleic acids enriched in nucleic acid sequences that are capable of binding to spike with increased affinity, whereby an aptamer that binds to spike is identified. In certain embodiments, the method further includes performing a slow off-rate enrichment process.

Sequence Identity: Sequence identity, as used herein, in the context of two or more nucleic acid sequences is a function of the number of identical nucleotide positions shared by the sequences (i.e., % identity=number of identical positions/total number of positions in the shorter of the two sequences being compared×100), taking into account the number of gaps, and the length of each gap that needs to be introduced to optimize alignment of two or more sequences.

The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley-Interscience (1987)). As used herein, when describing the percent identity of a nucleic acid, such as an aptamer, the sequence of which is at least, for example, about 95% identical to a reference nucleobase sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleobase sequence or anywhere between those terminal positions, interspersed either individually among nucleotides in the reference sequence or in one or more contiguous groups within the reference sequence.

SOMAmer: As used herein, a “SOMAmer” or Slow Off-Rate Modified Aptamer refers to an aptamer (including an aptamers comprising at least one nucleotide with a hydrophobic modification) with an off-rate (t_{1/2}) of ≥30 minutes, ≥60 minutes, ≥90 minutes, ≥120 minutes, ≥150 minutes, ≥180 minutes, ≥210 minutes, or ≥240 minutes. In some embodiments, SOMAmers are generated using the improved SELEX methods described in U.S. Pat. No. 7,947,447, entitled “Method for Generating Aptamers with Improved Off-Rates”.

Target Molecule: Target molecule (or target), as used herein, refers to any compound or molecule having a three-dimensional chemical structure other than a polynucleotide upon which an aptamer can act in a desirable manner. Non-limiting examples of a target molecule include a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc. Virtually any chemical or biological effector may be a suitable target. Molecules of any size can serve as targets. A target can also be modified in certain ways to enhance the likelihood or strength of an interaction between the target and the nucleic acid. A target may also include any minor variation of a particular compound or molecule, such as, in the case of a protein, for example, minor variations in its amino acid sequence, disulfide bond formation, glycosylation, lipidation, acetylation, phosphorylation, or any other manipulation or modification, including conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. In some embodiments, the target molecule is SARS-CoV-2 spike protein.

Therapeutically Effective Amount: As used herein, the term “therapeutically effective amount” generally means the amount necessary to ameliorate at least one symptom of a disorder or condition to be prevented, reduced, or treated as described herein. The phrase “therapeutically effective amount” as it relates to the aptamers of the present disclosure means the aptamer dosage that provides the specific pharmacological response for which the aptamer is administered in a significant number of individuals in need of such treatment. It is emphasized that a therapeutically effective amount of an aptamer that is administered to a particular individual in a particular instance will not always be effective in treating the conditions/diseases described herein, even though such dosage is deemed to be a therapeutically effective amount by those of skill in the art.

The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean+20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g., “or”) should be understood to mean either one, both, or any combination thereof of the alternatives

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### EXEMPLARY EMBODIMENTS

In some embodiments, an aptamer that binds a SARS-CoV-2 protein comprises a sequence that is at least 80%, at least 85%, at least 90%, at least 95%, at least 97%, at least 99%, or 100% identical to a sequence selected from SEQ ID NOs: 4, 6-20 and 28-122. In some embodiments, an aptamer that binds a SARS-CoV-2 protein comprises a sequence that is at least 80%, at least 85%, at least 90%, at least 95%, at least 97%, at least 99%, or 100% identical to a sequence selected from SEQ ID NOs:128-252, 254, 256 and 258.

In some embodiments, the aptamer is from 35 to 60 nucleotides in length, or from 35 to 50 nucleotides in length, or from 40 to 50 nucleotides in length. In some embodiments, the aptamer is from 25 to 60 nucleotides in length, or from 28 to 50 nucleotides in length, or from 30 to 50 nucleotides in length.

In some embodiments, the spike protein that the aptamer binds is a SARS-CoV-2 spike protein. In certain aspects, the aptamer binds the SARS-CoV-2 spike receptor binding domain (RBD). In certain aspects, the aptamer binds the SARS-CoV-2 spike S1. In certain aspects, the aptamer binds the SARS-CoV-2 spike S1 & S2 extracellular domain (ECD). In certain aspects, the aptamer binds the SARS-CoV-2 S1 & S2 ECD stable trimer. In certain aspects, the aptamer binds the SARS-CoV-2 spike S1 aspartic acid 614 to glycine mutant (D614G). In certain aspects, the aptamer binds the SARS-CoV-2 spike RBD asparagine 501 to tyrosine mutant (N501Y). In certain aspects, the aptamer binds the SARS-CoV-2 spike RBD glutamic acid 484 to lysine mutant (E484K). In certain aspects, the aptamer binds a SARS-CoV-2 spike protein of a variant SARS-CoV-2 selected from B1.1.7 variant (Alpha), B1.351 variant (Beta), ECD P.1 variant (Gamma), B.1.617.2 variant (Delta), and B.1.1.529 variant (Omicron).

In any of the embodiments described herein, the aptamer may be from 35 to 60 nucleotides in length, or from 35 to 50 nucleotides in length, or from 40 to 50 nucleotides in length. In any of the embodiments described herein, the aptamer may be from 25 to 60 nucleotides in length, or from 28 to 50 nucleotides in length, or from 30 to 50 nucleotides in length.

In some embodiments, the spike aptamer may include up to 100 nucleotides, up to 95 nucleotides, up to 90 nucleotides, up to 85 nucleotides, up to 80 nucleotides, up to 75 nucleotides, up to 70 nucleotides, up to 65 nucleotides, up to 60 nucleotides, up to 55 nucleotides, up to 50 nucleotides, up to 45 nucleotides, up to 40 nucleotides, up to 35 nucleotides, or up to 30 nucleotides.

In another aspect this disclosure, the spike aptamer may have a dissociation constant (K_{d}) for spike of about 10 nM or less. In another exemplary embodiment, the spike aptamer has a dissociation constant (K_{d}) for the spike protein of about 15 nM or less. In yet another exemplary embodiment, the spike aptamer has a dissociation constant (K_{d}) for the spike protein of about 20 nM or less. In yet another exemplary embodiment, the spike aptamer has a dissociation constant (K_{d}) for the spike protein of about 25 nM or less. In yet another exemplary embodiment, the spike aptamer has a dissociation constant (K_{d}) for the spike protein of about 30 nM or less. In yet another exemplary embodiment, the spike aptamer has a dissociation constant (K_{d}) for the spike protein of about 35 nM or less. In yet another exemplary embodiment, the spike aptamer has a dissociation constant (K_{d}) for the spike protein of about 40 nM or less. In yet another exemplary embodiment, the spike aptamer has a dissociation constant (K_{d}) for the spike protein of about 45 nM or less. In yet another exemplary embodiment, the spike aptamer has a dissociation constant (K_{d}) for the spike protein of about 50 nM or less. In yet another exemplary embodiment, the spike aptamer has a dissociation constant (K_{d}) for the spike protein in a range of about 2 pM to about 10 nM (or 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 50 pM, 60 pM, 70 pM, 80 pM, 90 pM, 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1000 pM, 2 nM, 3 nM, 4 nM, 5 nM, 6 nM, 7 nM, 8 nM, 9 nM or 10 nM). In yet another exemplary embodiment, the spike aptamer has a dissociation constant (K_{d}) for the spike protein in a range of at least 2 pM (or at least 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 50 pM, 60 pM, 70 pM, 80 pM, 90 pM, 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1000 pM, 2 nM, 3 nM, 4 nM, 5 nM, 6 nM, 7 nM, 8 nM, 9 nM or 10 nM). A suitable dissociation constant can be determined with a binding assay using a multi-point titration and fitting the equation y=(max−min)(Protein)/(K_{d}+Protein)+min as described in Example 2. It is to be understood that the determination of dissociation constants is highly dependent upon the conditions under which they are measured and thus these numbers may vary significantly with respect to factors such as equilibration time, etc.

In any of the embodiments described herein, the aptamer, nucleic acid molecule comprises nucleotides of DNA, RNA or a combination thereof.

SELEX

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to affect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e., the SOMAmer) for detecting and quantifying a non-nucleic acid target, thus allowing the wide variety of nucleic acid technologies, including amplification, to be applied to a broader range of desired targets, including protein targets.

Embodiments of the SELEX process in which the target is a peptide are described in U.S. Pat. No. 6,376,190, entitled “Modified SELEX Processes Without Purified Protein.” In the instant case, the target is the RIG-I-Protein.

### Chemical Modifications in Aptamers

Aptamers may contain modified nucleotides that improve its properties and characteristics. Non-limiting examples of such improvements include, in vivo stability, stability against degradation, binding affinity for its target, and/or improved delivery characteristics.

Examples of such modifications include chemical substitutions at the ribose and/or phosphate and/or base positions of a nucleotide. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

Specific examples of nucleosides comprising a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) as illustrated immediately below.

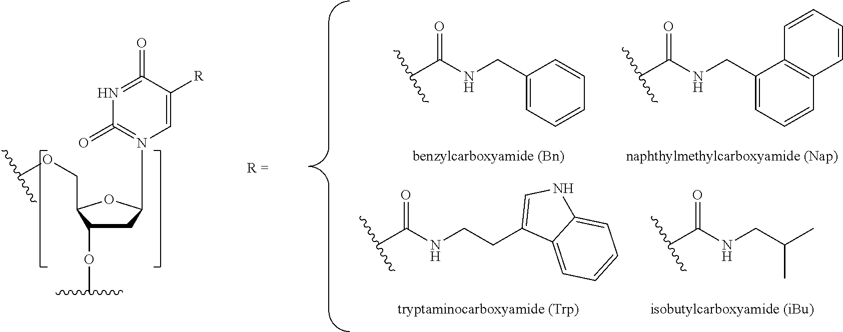

Chemical modifications of a C-5 modified pyrimidine can also be combined with, singly or in any combination, 2′-position sugar modifications, modifications at exocyclic amines, and substitution of 4-thiouridine and the like.

Representative C-5 modified pyrimidine containing nucleosides include. 5-(N-benzylcarboxy amide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethyl carboxyamide)-2′-fluorouridine or 5-(N-[1-(2,3-dihydroxypropyl)]carboxyamide)-2′-deoxyuridine), 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethyl carboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

If present, a modification to the nucleotide structure can be imparted before or after assembly of the polynucleotide. A sequence of nucleotides can be interrupted by non-nucleotide components. A polynucleotide can be further modified after polymerization, such as by conjugation with a labeling component.

Further, C-5 modified pyrimidine containing nucleotides include the following:

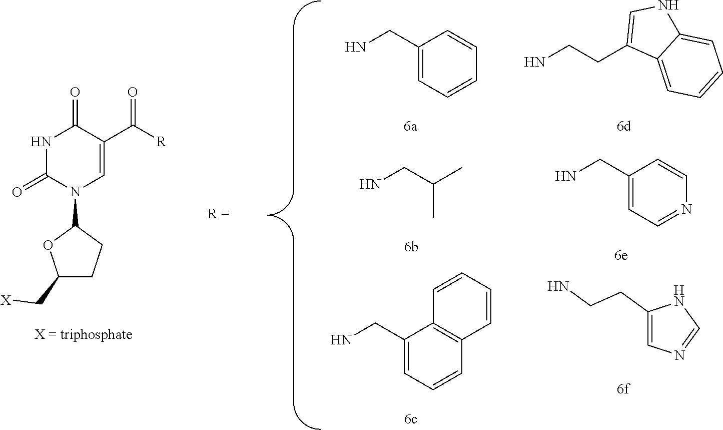

In some embodiments, the modified nucleotide confers nuclease resistance to the oligonucleotide. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleoside modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(0)S (“thioate”),

P(S)S (“dithioate”), (0)NR₂ (“amidate”), P(O)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

The present disclosure further provides for a formulation comprising a nucleic acid sequence selected from SEQ ID NOs: 4, 6-20 and 28-122. The present disclosure further provides for a formulation comprising a nucleic acid sequence selected from sequence selected from SEQ ID NOs:128-252, 254, 256 and 258.

In another aspect, each C-5 modified pyrimidine containing nucleoside is independently selected from:

5-(N-benzylcarboxamide)-2′-deoxyuridine (BndU), 5-(N-benzylcarboxamide)-2′-O-methyluridine (2′-OMe-Bn-U), 5-(N-benzylcarboxamide)-2′-fluorouridine (2′-F-Bn-U), 5-(N-phenethylcarboxamide)-2′-deoxyuridine (PEdU), 5-(N-phenethylcarboxamide)-2′-O-methyluridine (2′-OMe-PE-U), 5-(N-phenethylcarboxamide)-2′-fluorouridine (2′-F-PE-U), 5-(N-thiophenylmethylcarboxamide)-2′-deoxyuridine (ThdU), 5-(N-thiophenylmethylcarboxamide)-2′-O-methyluridine (2′-OMe-Th-U), 5-(N-thiophenylmethylcarboxamide)-2′-fluorouridine (2′-F-Th-U), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxamide)-2′-deoxyuridine (iBudU), 5-(N-isobutylcarboxamide)-2′-O-methyluridine (2′-OMe-iBu-U), 5-(N-isobutylcarboxamide)-2′-fluorouridine (2′-F-iBu-U), 5-(N-tyrosylcarboxamide)-2′-deoxyuridine (TyrdU), 5-(N-tyrosylcarboxamide)-2′-O-methyluridine (2′-OMe-Tyr-U), 5-(N-tyrosylcarboxamide)-2′-fluorouridine (2′-F-Tyr-U), 5-(N-3,4-methylenedioxybenzylcarboxamide)-2′-deoxyuridine (MBndU), 5-(N-3,4-methylenedioxybenzylcarboxamide)-2′-O-methyluridine (2′-OMe-MBn-U), 5-(N-3,4-methylenedioxybenzylcarboxamide)-2′-fluorouridine (2′-F-MBn-U), 5-(N-4-fluorobenzylcarboxamide)-2′-deoxyuridine (FBndU), 5-(N-4-fluorobenzylcarboxamide)-2′-O-methyluridine (2′-OMe-FBn-U), 5-(N-4-fluorobenzylcarboxamide)-2′-fluorouridine (2′-F-FBn-U), 5-(N-3-phenylpropylcarboxamide)-2′-deoxyuridine (PPdU), 5-(N-3-phenylpropylcarboxamide)-2′-O-methyluridine (2′-OMe-PP-U), 5-(N-3-phenylpropylcarboxamide)-2′-fluorouridine (2′-F—PP-U), 5-(N-imidizolylethylcarboxamide)-2′-deoxyuridine (ImdU), 5-(N-imidizolylethylcarboxamide)-2′-O-methyluridine (2′-OMe-Im-U), 5-(N-imidizolylethylcarboxamide)-2′-fluorouridine (2′-F-Im-U), 5-(N-tryptaminocarboxamide)-2′-deoxyuridine (TrpdU), 5-(N-tryptaminocarboxamide)-2′-O-methyluridine (2′-OMe-Trp-U), 5-(N-tryptaminocarboxamide)-2′-fluorouridine (2′-F-Trp-U), 5-(N—R-threoninylcarboxamide)-2′-deoxyuridine (ThrdU), 5-(N—R-threoninylcarboxamide)-2′-O-methyluridine (2′-OMe-Thr-U), 5-(N—R-threoninylcarboxamide)-2′-fluorouridine (2′-F-Thr-U), 5-(N-[1-(3-trimethylamonium) propyl]carboxamide)-2′-deoxyuridine chloride, 5-(N-[1-(3-trimethylamonium) propyl]carboxamide)-2′-O-methyluridine chloride, 5-(N-[1-(3-trimethylamonium) propyl]carboxamide)-2′-fluorouridine chloride, 5-(N-naphthylmethylcarboxamide)-2′-deoxyuridine (NapdU), 5-(N-naphthylmethylcarboxamide)-2′-O-methyluridine (2′-OMe-Nap-U), 5-(N-naphthylmethylcarboxamide)-2′-fluorouridine (2′-F-Nap-U), 5-(N-[1-(2,3-dihydroxypropyl)]carboxamide)-2′-deoxyuriine), 5-(N-[1-(2,3-dihydroxypropyl)]carboxamide)-2′-O-methyluridine), 5-(N-[1-(2,3-dihydroxypropyl)]carboxamide)-2′-fluorouridine), 5-(N-2-naphthylmethylcarboxamide)-2′-deoxyuridine (2NapdU), 5-(N-2-naphthylmethylcarboxamide)-2′-O-methyluridine (2′-OMe-2Nap-U), 5-(N-2-naphthylmethylcarboxamide)-2′-fluorouridine (2′-F-2Nap-U), 5-(N-1-naphthylethylcarboxamide)-2′-deoxyuridine (NEdU), 5-(N-1-naphthylethylcarboxamide)-2′-O-methyluridine (2′-OMe-NE-U), 5-(N-1-naphthylethylcarboxamide)-2′-fluorouridine (2′-F-NE-U), 5-(N-2-naphthylethylcarboxamide)-2′-deoxyuridine (2NEdU), 5-(N-2-naphthylethylcarboxamide)-2′-O-methyluridine (2′-OMe-2NE-U), 5-(N-2-naphthylethylcarboxamide)-2′-fluorouridine (2′-F-2NE-U), 5-(N-3-benzofuranylethylcarboxamide)-2′-deoxyuridine (BFdU), 5-(N-3-benzofuranylethylcarboxamide)-2′-O-methyluridine (2′-OMe-BF-U), 5-(N-3-benzofuranylethylcarboxamide)-2′-fluorouridine (2′-F—BF-U), 5-(N-3-benzothiophenylethylcarboxamide)-2′-deoxyuridine (BTdU), 5-(N-3-benzothiophenylethylcarboxamide)-2′-O-methyluridine (2′-OMe-BT-U), 5-(N-3-benzothiophenylethylcarboxamide)-2′-fluorouridine (2′-F-BT-U), 5-(N-benzylcarboxamide)-2′-deoxycytidine (BndC); 5-(N-benzylcarboxamide)-2′-O-methylcytidine (2′-OMe-Bn-C); 5-(N-benzylcarboxamide)-2′-fluorocytidine (2′-F-Bn-C); 5-(N-2-phenylethylcarboxamide)-2′-deoxycytidine (PEdC); 5-(N-2-phenylethylcarboxamide)-2′-O-methylcytidine (2′-OMe-PE-C); 5-(N-2-phenylethylcarboxamide)-2′-fluorocytidine (2′-F-PE-C); 5-(N-3-phenylpropylcarboxamide)-2′-deoxycytidine (PPdC); 5-(N-3-phenylpropylcarboxamide)-2′-O-methylcytidine (2′-OMe-PP-C); 5-(N-3-phenylpropylcarboxamide)-2′-fluorocytidine (2′-F—PP-C); 5-(N-1-naphthylmethylcarboxamide)-2′-deoxycytidine (NapdC); 5-(N-1-naphthylmethylcarboxamide)-2′-O-methylcytidine (2′-OMe-Nap-C); 5-(N-1-naphthylmethylcarboxamide)-2′-fluorocytidine (2′-F-Nap-C); 5-(N-2-naphthylmethylcarboxamide)-2′-deoxycytidine (2NapdC); 5-(N-2-naphthylmethylcarboxamide)-2′-O-methylcytidine (2′-OMe-2Nap-C); 5-(N-2-naphthylmethylcarboxamide)-2′-fluorocytidine (2′-F-2Nap-C); 5-(N-1-naphthyl-2-ethylcarboxamide)-2′-deoxycytidine (NEdC); 5-(N-1-naphthyl-2-ethylcarboxamide)-2′-O-methylcytidine (2′-OMe-NE-C); 5-(N-1-naphthyl-2-ethylcarboxamide)-2′-fluorocytidine (2′-F-NE-C); 5-(N-2-naphthyl-2-ethylcarboxamide)-2′-deoxycytidine (2NEdC); 5-(N-2-naphthyl-2-ethylcarboxamide)-2′-O-methylcytidine (2′-OMe-2NE-C); 5-(N-2-naphthyl-2-ethylcarboxamide)-2′-fluorocytidine (2′-F-2NE-C); 5-(N-tyrosylcarboxamide)-2′-deoxycytidine (TyrdC); 5-(N-tyrosylcarboxamide)-2′-O-methylcytidine (2′-OMe-Tyr-C); and 5-(N-tyrosylcarboxamide)-2′-fluorocytidine (2′-F-Tyr-C).

In another aspect, the C-5 modified pyrimidine containing nucleoside is independently selected from:

5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In another aspect, the C-5 modified pyrimidine containing nucleoside is 5-(N-3-phenylpropylcarboxyamide)-2′-deoxyuridine (PPdU).

In another aspect, the two or more nucleic acid molecules of the formulation are each, independently, from 35 to 60 nucleotides in length, or from 35 to 50 nucleotides in length, or from 40 to 50 nucleotides in length; or further comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 additional nucleotides.

### Pharmaceutical Compositions Comprising Aptamers

In some embodiments, pharmaceutical compositions comprising at least one aptamer described herein and at least one pharmaceutically acceptable carrier are provided. Suitable carriers are described in “Remington: The Science and Practice of Pharmacy, Twenty-first Edition,” published by Lippincott Williams & Wilkins, which is incorporated herein by reference.

The aptamers described herein can be utilized in any pharmaceutically acceptable dosage form, including, but not limited to, injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the aptamers described herein can be formulated: (a) for administration selected from any of intravitreal, oral, pulmonary, intravenous, intraarterial, intrathecal, intra-articular, rectal, ophthalmic, colonic, parenteral, intracisternal, intravaginal, intraperitoneal, local, buccal, nasal, and topical administration; (b) into a dosage form selected from any of liquid dispersions, gels, aerosols, ointments, creams, tablets, sachets and capsules; (c) into a dosage form selected from any of lyophilized formulations, dry powders, fast melt formulations, controlled release formulations, delayed release formulations, extended release formulations, pulsatile release formulations, and mixed immediate release and controlled release formulations; or (d) any combination thereof.

Solutions or suspensions used for parenteral, intradermal, or subcutaneous application can comprise one or more of the following components: (1) a sterile diluent such as water for injection, saline solution, fixed oils, polyethylene glycols, glycerine, propylene glycol or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates or phosphates; and (5) agents for the adjustment of tonicity such as sodium chloride or dextrose. The pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition should be sterile and should be fluid to the extent that easy syringability exists. The pharmaceutical composition should be stable under the conditions of manufacture and storage and should be preserved against the contaminating action of microorganisms such as bacteria and fungi.

The term “stable”, as used herein, means remaining in a state or condition that is suitable for administration to a subject.

The carrier can be a solvent or dispersion medium, including, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, and inorganic salts such as sodium chloride, in the composition. Prolonged absorption of the injectable compositions can be brought about by including in the composition an agent which delays absorption, for example, aluminum monostearate and gelatin.

Sterile injectable solutions can be prepared by incorporating the active reagent (e.g., an aptamer) in an appropriate amount in an appropriate solvent with one or a combination of ingredients enumerated above, as desired, followed by filtered sterilization. Generally, dispersions are prepared by incorporating at least one aptamer into a sterile vehicle that contains a basic dispersion medium and any other desired ingredient. In the case of sterile powders for the preparation of sterile injectable solutions, exemplary methods of preparation include vacuum drying and freeze-drying, both of which will yield a powder of an aptamer plus any additional desired ingredient from a previously sterile-filtered solution thereof.

In some embodiments, an aptamer is formulated for intravitreal injection. Suitable formulations for intravitreal administration are described, e.g., in “Remington: The Science and Practice of Pharmacy, Twenty-first Edition,” published by Lippincott Williams & Wilkins. Ocular drug delivery is discussed, e.g., in Rawas-Qalaji et al. (2012) Curr. Eye Res. 37: 345; Bochot et al. (2012) J. Control Release 161:628; Yasukawa et al. (2011) Recent Pat. Drug Deliv. Formul. 5: 1; and Doshi et al. (2011) Semin. Ophthalmol. 26: 104. In some embodiments, a pharmaceutical composition comprising an aptamer is administered by intravitreal injection once per week, once per two weeks, once per three weeks, once per four weeks, once per five weeks, once per six weeks, once per seven weeks, once per eight weeks, once per nine weeks, once per 10 weeks, once per 11 weeks, once per 12 weeks, or less often than once per 12 weeks.

Oral compositions generally include an inert diluent or an edible carrier. They can be enclosed, for example, in gelatin capsules or compressed into tablets. For the purpose of oral therapeutic administration, the aptamer can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

For administration by inhalation, the compounds are delivered in the form of an aerosol spray from a pressured container or dispenser that contains a suitable propellant, e.g., a gas such as carbon dioxide, a nebulized liquid, or a dry powder from a suitable device. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams, as generally known in the art. The reagents can also be prepared in the form of suppositories (e.g., with conventional suppository bases such as cocoa butter and other glycerides) or retention enemas for rectal delivery.

In some embodiments, an aptamer is prepared with a carrier that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of an aptamer may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

In some cases, it may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of an aptamer calculated to produce the desired therapeutic effect in association with the required pharmaceutical carrier. The specification for the dosage unit forms of aptamers described herein are dictated by and directly dependent on the characteristics of the particular aptamer and the particular therapeutic effect to be achieved, and the limitations inherent in the art of compounding such an active agent for the treatment of individuals.

Pharmaceutical compositions comprising at least one aptamer can include one or more pharmaceutical excipients. Examples of such excipients include, but are not limited to, binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and cross-linked polyvinylpyrrolidone, microcrystalline cellulose, such as Avicel PH101 and Avicel PHI 02, silicified microcrystalline cellulose (ProSolv SMCC™), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, and including magnesium stearate, colloidal silicon dioxide, such as Aerosil 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel PH101 and Avicel PHI 02; lactose such as lactose monohydrate, lactose anhydrous, and Pharmatose DCL21; dibasic calcium phosphate such as Emcompress; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In various embodiments, the formulations described herein are substantially pure. As used herein, “substantially pure” means the active ingredient (e.g., an aptamer) is the predominant species present (i.e., on a molar basis it is more abundant than any other individual species in the composition). In some embodiments, a substantially purified fraction is a composition wherein the active ingredient comprises at least about 50 percent (on a molar basis) of all macromolecular species present. Generally, a substantially pure composition will include more than about 80% of all macromolecular species present in the composition. In various embodiments, a substantially pure composition will include at least about 85%, at least about 90%, at least about 95%, or at least about 99% of all macromolecular species present in the composition. In various embodiments, the active ingredient is purified to homogeneity (contaminant species cannot be detected in the composition by conventional detection methods) wherein the composition consists essentially of a single macromolecular species.

### Kits Comprising Aptamers

The present disclosure provides kits comprising any of the aptamers described herein. Such kits can comprise, for example, (1) at least one aptamer; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial or similar apparatus for holding and/or mixing the kit components; and (3) delivery apparatus.

The following examples are provided to illustrate certain particular features and/or embodiments. These examples should not be construed to limit the disclosure to the particular features or embodiments described.

### EXAMPLES

The following examples are presented in order to more fully illustrate some embodiments of the invention. They should in no way be construed, however, as limiting the broad scope of the invention. Those of ordinary skill in the art can readily adopt the underlying principles of this discovery to design various compounds without departing from the spirit of the current invention.

Throughout the Figures and Examples, aptamers may be identified by an “Aptamer ID” or a corresponding “SL” number as shown in Table 1 below.

TABLE 1

| | Corresponding Identification in |
|---|---|
| Aptamer ID | Certain Figures and Examples |
| 26876-3_18 | SL1114_18 |
| 26876-3_4 | SL1114 |
| 26876-3_36 | SL1114_36 |
| 26876-13_18 | SL1115_18 |
| 26876-13_26 | SL1115_26 |
| 26874-29_20 | SL1111_20 |
| 26874-29_4 | SL1111 |
| 26874-29_354 | SL1111_354 |
| 26860-16_18 | SL1108_18 |
| 26860-16_3 | SL1108 |
| 26860-16_28 | SL1108_28 |
| 26860-75_18 | SL1107_18 |
| 26860-75_3 | SL1107 |
| 26860-75_26 | SL1107_26 |
| 26845-63_3 | SL1104 |
| OH-26846-54_3 | SL1105 |
| OH-26847-272_3 | SL1106 |
| OH-26860-26_3 | SL1109 |
| OH-26863-76_3 | SL1110 |
| OH-26875-46_4 | SL1112 |
| OH-26875-150_4 | SL1113 |
| OH-26876-40_4 | SL1116 |

### Example 1: Selection and Identification of Aptamers Having Binding Specificity to Coronavirus Proteins

This example provides the representative method for the selection and production of DNA aptamers to the coronavirus spike protein.

### Preparation of Candidate Mixture

A candidate mixture of partially randomized ssDNA oligonucleotides was prepared by polymerase extension of a DNA primer annealed to a biotinylated ssDNA template (shown in Table 2 below). The candidate mixture contained a 30-nucleotide randomized cassette containing dATP, dGTP, Nap-dCTP and 5-(p-hydroxyphenethyl)-1-aminocarbonyl deoxyuridine triphosphate (Tyr-dUTP).

TABLE 2
Sequences of Template and Primers

| Oligo- | | SEQ |
|---|---|---|
| nucleotide | | ID |
| Designation | Sequence (5′ → 3′) | NO: |
| Template 1 | AB′AB′ TTT TTT TTC | 1 |
| | TCT CTT CCT CCC TTT | |
| | TTC CTT C- (N)₃₀-CAA | |
| | GGT AGG TCG GCG | |
| | TCA CT | |
| Primer 1 | ATA TAT ATA GTG | 2 |
| | ACG CCG ACC TAC | |
| | CTT G | |
| Primer 2 | AB′AB′ TTT TTT TTC | 3 |
| | TCT CTT CCT CCC TTT | |
| | TTC CTT C | |

B′ = biotin

One thousand seventy five microliters of a 50% slurry of Streptavidin Plus UltraLink Resin (PIERCE) was washed once with 10 mL of 20 mM sodium hydroxide (NaOH), twice with 10 mL of SB18T0.01 (40 mM HEPES (4-(2-Hydroxyethyl)piperazine-1-ethanesulfonic acid) buffer adjusted to pH 7.5 with NaOH, 102 mM NaCl, 5 mM KCl, 5 mM MgCl2 and 0.01% TWEEN 20) and twice with 10 mL of 16 mM NaCl. Resin was resuspended in a final volume of 1.075 mL 16 mM NaCl. Forty three nanomoles of template 1 (SEQ ID NO: 1) possessing two biotin residues (designated as B′ in the sequence) and 30 randomized positions (designated as N30 in the sequence) were added to the washed UltraLink SA beads and rotated at room temperature for 90 minutes. The beads were then washed three times with SB18T0.01 and two times with 16 mM NaCl. Between each wash, the beads were recovered by centrifugation. The beads, now containing the captured template, were suspended in 2.15 mL of extension reaction buffer [containing 64.5 nmol of primer 1 (SEQ ID NO: 2), 1×SQ20 buffer (120 mM Tris-HCl, pH7.8, 10 mM KCl, 7 mM MgSO₄, 6 mM (NH₄)₂SO₄, 0.001% BSA and 0.1% Triton X-100), 269 units of KOD XL DNA Polymerase (EMD MILLIPORE), and 1 mM each of dATP, dGTP, Nap-dCTP and Tyr-dUTP. The beads were allowed to incubate at 68° C. for 2 hours. The beads were then washed one time with SB18T0.01 and three times with 16 mM NaCl. The aptamer library was eluted from the beads with 21 mL of 20 mM NaOH. The eluted library and immediately neutralized with 320 μL of 1N HCl and 100 μL HEPES pH 7.5 and 2 μL 10% TWEEN-20. The library was concentrated with an AMICON Ultracel YM-10 filter to approximately 0.62 mL and the concentration of library determine by ultraviolet absorbance spectroscopy.

### Immobilization of Target Protein

His-tagged generated target protein was immobilized on His-tag Dynabeads (Thermo Fisher) paramagnetic beads (MyOne SA, Invitrogen, or hereinafter referred to as His beads) for SELEX (Rounds 1 through 10). Beads (40 mgs) were prepared by washing three times with 20 mL of SB18T0.01. Finally, the beads were suspended at 2.5 mgs/mL in SB18T0.01 and stored at 4° C. until use.

Aptamer Selection with Slow Off-Rate Enrichment Process

A total of ten rounds of the SELEX process were completed with selection for affinity and slow off-rate. Prior to each round a counter selection was performed to reduce background and to reduce the likelihood of obtaining aptamers with nonspecific binding to protein. Counter selections were performed as follows.

For round 1, 100 μL of the DNA candidate mixture containing approximately 1 nmole of DNA in SB18T0.01 was heated at 95° C. for 5 minutes and then cooled to 70° C. for 5 minutes, then to 48° C. for 5 minutes and then transferred to a 37° C. block for 5 minutes. The sample was then combined with 10 μL of protein competitor mixture (0.1% HSA, 10 pM casein, and 10 pM prothrombin in SB18T0.01), and 0.025 mg (10 μL) of His beads coated with HEXA-His (Anaspec, catalog #24420) and incubated at 37° C. for 10 minutes with mixing. Beads were removed by magnetic separation.

For Rounds 2-10, a 65 μL aliquot of the DNA candidate mixture obtained from the previous round (65% of eDNA obtained from previous round) was mixed with 16 μL of 5×SB18T0.01. The sample was heated to 95° C. for 3 minutes and cooled to 37° C. at a rate of 0.1° C./second. The sample was then combined with 9 μL of protein competitor mixture (0.1% HSA, 10 μM casein, and 10 μM prothrombin in SB18T0.01), and 0.025 mg (10 uL) His beads and incubated at 37° C. for 10 minutes with mixing. Beads were removed by magnetic separation.

Following the first counter selection the target protein was pre-immobilized on His beads for the Round 1 selection process. To accomplish this, 0.125 mg of protein His beads were mixed with 50 pmoles of target protein and incubated for 30 minutes at 37° C. Unbound target was removed by washing the beads with SB18T0.01. The counter-selected-DNA candidate mixture (100 μL) was added to the beads and incubated at 37° C. for 60 minutes with mixing. No slow off-rate enrichment process was employed in the first round and beads were simply washed 5 times with 100 μL SB18T0.01. Following the washes, the bound aptamer was eluted from the beads by adding 170 μL of 2 mM NaOH, and incubating at 37° C. for 5 minutes with mixing. The aptamer-containing-eluate (170 μL) was transferred to a new tube after magnetic separation of the beads and the solution neutralized by addition of 40 μL of neutralization buffer (500 mM Tris-HCl pH 7.5, 8 mM HCl).

For Rounds 2-10, selections were performed with the DNA candidate mixture and target protein as described below while, in parallel, an identical selection was performed with the DNA candidate mixture, but without the target protein. Comparison of the Ct values obtained from PCR for the sample with target protein (signal S) and sample without target protein (background B) were used as a guide to reduce the target concentration in the next round. If the delta Ct value was greater than 4, but less than 8, the target protein was reduced three-fold in the next round. If the delta Ct value was greater than 8, the target was reduced 10-fold in the next round.

For Round 2, labeled target protein (5 pmoles in 10 μL) was mixed with 40 μL of counter selected DNA candidate mixture and incubated at 37° C. for 15 minutes. A slow off-rate enrichment process was begun by adding 50 μL of 10 mM dextran sulfate followed by the immediate addition of 0.0125 mg of His beads. This was allowed to incubate for 15 minutes at 37° C. with mixing. Beads were then washed 5 times with 100 μL of SB18T0.01. The aptamer strand was eluted from the beads by adding 100 μL of sodium perchlorate, and incubating at 37° C. for 10 minutes with mixing. Beads were removed by magnetic separation and 100 μL of aptamer eluate was transferred to a new tube.

Round 3 through 10 were performed as described for Round 2 except the amount of target protein was 5 pmoles for rounds 3 and 4, 1.6 pmoles for round 5, 0.5 pmoles for round 6, 0.16 pmoles for rounds 7 and 8, 0.05 pmoles for rounds 9 and 10. The dextran sulfate was added 15 minutes (rounds 3 and 4), 30 minutes (rounds 5 and 6), 45 minutes (rounds 8 through 10) prior to the addition of His beads.

Rounds 9 through 10, protein and counter selected DNA candidate mixture incubated at 37° C. for 10 seconds prior to the slow off-rate enrichment process.

For rounds 2 through 10, following the perchlorate elution, 100 μL of the aptamer eluate was captured with 0.0625 mg (25 μL) of SA beads pre-bound with primer 2 (SEQ ID NO: 3), herein after referred to as Primer Beads, and incubated at 50° C. for 10 minutes with shaking followed by a 25° C. incubation for 10 minutes with shaking. The beads were washed 2 times with 100 μL SB18T0.01 and 1 time with 16 mM NaCl. Bound aptamer was eluted with 120 μL water and incubated at 75° C. for 2 minutes. Beads were removed by magnetic separation and 120 μL of aptamer eluate was transferred to a new tube. Primer beads were prepared by resuspending 15 mg SA beads (1.5 mL of 10 mg/mL SA beads washed once with 2 mL 20 mM NaOH, twice with 2 mL SB18T0.01) in 0.5 mL 1 M NaCl, 0.01% tween-20 and adding 7 nmoles primer 2 (SEQ ID NO: 3). The mixture was incubated at 37° C. for 1 hour. Following incubation, the beads were washed 2 times with 1 mL SB18T0.01 and 2 times with 1 mL 16 mM NaCl. Beads were resuspended to 2.5 mg/ml in 5 M NaCl, 0.01% tween-20.

### Aptamer Amplification and Purification

Selected aptamer DNA from each round was amplified and quantified by QPCR. 48 μL DNA was added to 12 μL QPCR Mix (10×KOD DNA Polymerase Buffer; Novagen #71157, diluted to 5×, 25 mM MgCl2, 5 pM forward PCR primer (Primer 1, SEQ ID NO:2), 5 pM biotinylated reverse PCR primer (Primer 2, SEQ ID NO:3), 5×SYBR Green I, 0.075 U/μL KOD XL DNA Polymerase, and 1 mM each dATP, dCTP, dGTP, and dTTP) and thermal cycled in a Bio-Rad MyIQ QPCR instrument with the following protocol: 1 cycle of 96° C. for 15 seconds and 68° C. for 30 minutes; followed by 25 cycles of 96° C. for 15 seconds, 68° C. for 1 minute. Quantification was done with the instrument software and the number of copies of DNA selected, with and without target protein, was compared to determine signal/background ratios.

Following amplification, the PCR product was captured on SA beads via the biotinylated antisense strand. 25 mL SA beads (10 mg/mL) were washed once with 25 mL 20 mM NaOH, twice with 25 mL SB18T0.01, resuspended in 25 mL SB18T0.01, and stored at 4° C. 25 μL SA beads (10 mg/mL in SB18T0.01) were added to 50 μL double-stranded QPCR products and incubated at 25° C. for 5 minutes with mixing. The “sense” strand was eluted from the beads by adding 100 μL 20 mM NaOH, and incubating at 25° C. for 1 minute with mixing. The eluted strand was discarded and the beads were washed 2 times with SB18T0.01 and once with 16 mM NaCl.

Aptamer sense strand containing Tyr-dUTP and Nap-dCTP was prepared by primer extension from the immobilized antisense strand. The beads were suspended in 40 μL primer extension reaction mixture (1× Primer Extension Buffer (120 mM Tris-HCl pH 7.8, 10 mM KCl, 7 mM MgSO₄, 6 mM (NH₄)2SO₄, 0.1% TRITON X-100 and 0.001% bovine serum albumin), 4 μM forward primer (Primer 1, SEQ ID NO: 2), 0.5 mM each dATP, Nap-dCTP, dGTP, and Tyr-dUTP, and 0.075 U/μL KOD XL DNA Polymerase) and incubated at 68° C. for 45 minutes with mixing. The beads were washed 2 times with SB18T0.01, 1 time with 16 mM NaCl and the aptamer strand was eluted from the beads by adding 85 μL of 20 mM NaOH, and incubating at 37° C. for 2 minute with mixing. 83 μL aptamer eluate was transferred to a new tube after magnetic separation, neutralized with 20 μL of 80 mM HCl, buffered with 5 μL of 0.1 M HEPES, pH 7.5.

### Selection Stringency and Feedback

The relative target protein concentration of the selection step was lowered each round in response to the QPCR signal (A Ct) following the rule below:

If ΔCt<4, [P]₍ᵢ₊₁₎=[P]₍ᵢ₎

If 4≤ΔCt<8, [P]₍ᵢ₊₁₎=[P]₍ᵢ₎/3.2

If ΔCt≥8, [P]₍ᵢ₊₁₎=[P]₍ᵢ₎/10
Where [P]=protein concentration and i=current round number.

After each selection round, the convergence state of the enriched DNA mixture was determined. 10 μL double-stranded QPCR product was diluted to 200 μL with 4 mM MgCl2 containing 1×SYBR Green I. Samples were analyzed for convergence using a Cot analysis which measures the hybridization time for complex mixtures of double stranded oligonucleotides. Samples were thermal cycled with the following protocol: 3 cycles of 98° C. for 1 minute, 85° C. for 1 minute; 2 cycles of 98° C. for 1 minute, then 85° C. for 30 minutes. During the 30 minutes at 85° C., fluorescent images were measured at 5-second intervals. The fluorescence intensity was plotted as a function of the logarithm of time, and an increased rate of hybridization with each SELEX round was observed, indicating sequence convergence.

### Enriched Pool Sequencing & Aptamer Identification

After 10 rounds of SELEX, the converged pools from round 8 and round 10 were sequenced. Sequence preparation was performed as follows. The SELEX pool was converted to AGCT by PCR. To create a pooled sequencing template, AGCT samples were amplified again in PCR1 by real time qPCR using SELEX library-specific primers containing dual unique barcode and partial Illumina adaptor sequences. PCR1 cycling was observed in real time and stopped manually when all samples were in exponential amplification having exceeded 1,000 RFU (before plateau). Individual PCR1 products were next visualized on a 10% TBE Urea gel (Invitrogen) to confirm product size and purity. PCR1 products were next purified twice using Ampure beads (Beckman Coulter) and pooled to achieve an equimolar mixture based on quantification by each sample's qPCR1 endpoint RFU. The pooled PCR1 mixture was amplified in PCR2 with a single universal primer pair to complete Illumina adaptor addition, and the PCR2 pooled product purified by Ampure beads and visualized on a 10% TBE Urea gel for purity. The final sample (sequencing template) was quantified using Quant-iT™ PicoGreen® dsDNA Reagent (Life Technologies) and Qubit (ThermoFisher Scientific) assays and a Tape Station (Agilent) analysis for purity. Samples were sequenced on an iSeq Instrument (Illumina). Raw sequence data was filtered computationally for valid dual barcode combinations resulting in 42,060-126,140 valid sequences per pool, 1,000 of which were randomly selected and analyzed for convergence using custom software that determines sequence counts/copy number and identifies common convergence patterns using a local-alignment algorithm. Sequences with the greatest representation/copy number in the pool and at least one sequence from every convergence pattern were chosen for further characterization.

### Aptamer Synthesis

For determination of the binding potential, individual aptamers were prepared by solid phase synthesis. The modified deoxyuridine-5-carboxamide amidite reagent used for solid-phase synthesis was prepared by: condensation of 5′-O-(4,4′-dimethoxytrityl)-5-trifluoroethoxycarbonyl-2′-deoxyuridine (Nomura et al. (1997) Nucl. Acids Res. 25:2784) with the appropriate [1-naphthylmethylamine] primary amine (RNH₂, 1.2 eq; Et₃N, 3 eq.; acetonitrile; 60° C.; 4 h); 3′-O-phophitidylation with 2-cyanoethyl-N,N-diisopropylchlorophosphoramidite (1.2 eq.; iPr₂EtN, 3 eq.; CH₂Cl₂; −10 to 0° C.; 4 h); and purification by flash chromatography on neutral silica gel (Still, et al. (1978) J. Org. Chem. 43:2923). Aptamers were prepared by solid phase synthesis using the phosphoramidite method (Beaucage and Caruthers (1981) Tetrahedron Lett. 22:1859) with some adjustments to the protocol to account for the unique base modifications described herein. Detritylation was accomplished with 10% dichloroacetic acid in toluene for 45 seconds; coupling was achieved with 0.1 M phosphoramidites in 1:1 acetonitrile:dichloromethane activated by 5-benzylmercaptotetrazole and allowed to react 3 times for 5 minutes; capping and oxidation were performed according to instrument vendor recommendations. Deprotection was affected with gaseous ammonia or methylamine under optimized pressure, time, and temperature in a Parr stainless steel reactor. Products were eluted with dI water into suitable 96-well plates, statistically sampled (N+1) for LCMS characterization, quantified by UV spectrophotometry, and tested for protein binding affinity in buffered aqueous solution.

### Example 2: Selection and Identification of Aptamers Having Binding Specificity to Coronavirus Proteins

This example provides the representative method for the selection and production of DNA aptamers with diverse compositions to bind multiple epitopes of the SARS-CoV-2 spike protein.

To obtain coverage of a wide range of epitopes, five different monomeric constructs of the spike protein were targeted: four from SARS-CoV-2 (S1 domain, S2 domain, both S1 and S2 domains (the entire ectodomain), and the receptor binding domain (RBD)), and one (S1 domain) from the original SARS coronavirus (SARS-CoV), which was used in alternating rounds of selection with the S1 domain from SARS-CoV-2 using a toggle strategy to obtain ligands that recognize conserved epitopes. For each of these five proteins, separate SELEX experiments were performed with five chemically diverse random DNA libraries containing different 5-position modifications on pyrimidine bases: 1-naphthylmethyl (Nap), 3-indole-2-ethyl (Trp), phenylbenzyl (PBn) or diphenylpropyl (DPP) side chains as single modifications on all dU residues within a 40N random region, or double modifications comprising hydroxyphenyl-2-ethyl (Tyr) side chain on all dU residues and Nap side chain on all dC residues within a 30N random region.

### Selection of SOMAmer Reagents

SOMAmer reagents targeting recombinant spike proteins, spike S1 and S2 ECD (SinoBiological, Cat #40589-V08B1), spike S1 subunit SARS-CoV-1 (SinoBiological, Cat #40150-V08B1), spike S2 ECD (SinoBiological Cat #40590-V08B), spike S1 Receptor Binding Domain (RBD) (Creative Biomart, Cat #Spike-190V) and spike S1 subunit SARS-CoV-2 (Creative Biomart, Cat #Spike-191V) were discovered via the SELEX process^{17,27} from libraries containing either a 40-nucleotide random region in which dT was substituted with TrpdU, NapdU, PBndU, or DPPdU, or a 30-nucleotide random region in which dT was substituted for TyrdU and dC was substituted for NapdC. The 40-nucleotide random region was flanked by a 20-nucleotide forward primer (5′ GGTCGGGCACACTACGCATC) (SEQ ID NO: 260) and a 21-nucleotide reverse primer (5′ GGGAAGAGAAAGGAGAAGAAG) (SEQ ID NO: 261) while the 30-nucleotide library random region was flanked by a 20-nucleotide forward primer (5′ GGTCGGGCACACTACGCATC) (SEQ ID NO: 262) and a 21-nucleotide reverse primer (5′ GGGAAGAGAAAGGAGAAGAAG) (SEQ ID NO: 263). The total length of each SELEX library was either 89 (40N library) or 79 (30N library) nucleotides including an eight-nucleotide poly-dA region on the 3′ end. SELEX was performed in 1XSB18T buffer (40 mM HEPES, pH 7.5, 102 mM NaCl, 5 mM KCl, 5 mM MgCl₂, 0.05% Tween-20. To preferentially select for modified aptamers with slow off-rates, a kinetic challenge was included, whereby protein/DNA complexes were incubated at 37° C. in the presence of the polyanionic competitor, dextran sulfate. The duration of the kinetic challenge was increased from 30 seconds in round 2 to 15 minutes in rounds 3-4, 30 minutes in rounds 5-6 and 45 minutes in rounds 7-8. Simultaneous to the polyanionic competitor challenge, the protein concentrations were lowered.

SELEX was executed as follows. SELEX libraries were heat/cooled at 95° C. for 5 minutes, and then cooled to 37° C. at 0.1° C./sec. Following heat/cool, libraries were incubated at 37° C. for 10 minutes with Protein Competitor Buffer (10 uM prothrombin, 10 uM casein, 0.01% human serum albumin) and 25 ug Hexa-His bound His-tag Dyna beads (Invitrogen, Cat #101-04D) for counter selection. After 10 minutes the supernatant was removed and transferred to a clean plate. For round one of SELEX only, 50 pmoles of protein was immobilized 125 ug His-tag Dyna beads and transferred to the counter selected library and incubated at 37° C. for one hour with shaking. For rounds 2-8, proteins were in solution during library incubation at 37° C. for 10 minutes with no shaking. Prior to capturing protein-DNA complexes with His-tag Dyna beads for rounds 2-8, the kinetic challenge was initiated with 5 mM dextran sulfate (final concentration). Beads were then washed five times in 1XSB18T buffer. Elution was achieved with 2 mM NaOH and neutralized with HCl and buffered to pH 7.5 with Tris (round 1) or perchlorate elution buffer (40 mM PIPES pH 6.8, 1 mM EDTA, 0.05% Triton) followed by QPCR for 25 cycles, or until samples plateaued. After QPCR, DNA was captured on Dynal MyOne streptavidin beads and the sense strand eluted with 20 mM NaOH and discarded. The modified nucleotide sense strand was prepared with the appropriate nucleotide composition by primer extension from the immobilized antisense strand. After 8 rounds of SELEX, the converged pools were sequenced.

As some intended uses of spike protein SOMAmer reagents may require fast on-rates, two additional rounds of SELEX were performed subsequent to round 8, as described above with the following exceptions. The target proteins were incubated with the SELEX libraries for 10 seconds before the addition of polyanionic competitor dextran sulfate. Additionally, all protein concentrations were reduced 0.5 log from their round 8 concentrations. Round 10 pools were sequenced and compared to the round 8 sequencing results.

### Modified Aptamer Synthesis

The modified deoxyuridine-5-carboxamide phosphoramidite reagents used for solid-phase synthesis were prepared by: condensation of 5′-O-(4,4′-dimethoxytrityl)-5-trifluoroethoxycarbonyl-2′-deoxyuridine with the appropriate primary amine; 3′-O-phosphitylation with 2-cyanoethyl-N,N,N′,N′-tetraisopropylphosphene; and purification by flash chromatography on neutral silica gel as described in Example 1.

The modified deoxycytidine-5-carboxamide phosphoramidite reagents used for solid-phase synthesis were prepared using a four-step synthetic strategy from 5′-OH-5-iodo-2′-deoxycytidine condensed with the appropriate primary amine; N-protection of the 3-amino position of cytidine; 0-protection of the 5′ alcohol with 5,5′-dimethowxytrityl; 3′-O-phosphitylation with 2-cyanoethyl-N,N,N′,N′-tetraisopropylphosphene; and purification by flash chromatography on neutral silica gel as described in Example 1.

Modified aptamers were produced by conventional solid phase oligonucleotide synthesis using the phosphoramidite method. (Beaucage, S. L. C., et al. Tetrahedron Letters 22, 1859-1862 (1981). The aptamers were synthesized at the 50 nmole scale in a plate-based system on a Mermade 192×DNA synthesizer with some adjustments to the protocol to account for the unique base modifications contained therein. Detritylation was accomplished with 10% dichloroacetic acid in toluene; coupling was achieved with 0.1 M phosphoramidites in straight acetonitrile or a mix of acetonitrile:dichloromethane activated by 5-benzylmercaptotetrazole and allowed to react 3 times; capping and oxidation were performed according to instrument vendor recommendations. The resulting oligonucleotides were deprotected and cleaved from the controlled pore glass (CPG) by placing the plate containing synthesis columns in a reactor and incubating them with gaseous methylamine using an optimized time, pressure and temperature.

Deprotection by-products were removed by washing the columns containing the cleaved aptamers and spent CPG with high organic washes drawn through the bed via a vacuum manifold, then dried thoroughly. The desired product was next eluted from the CPG using high purity water via the vacuum manifold and collected into Matrix tubes. The resulting products underwent no additional purification and were characterized by LCMS using and Agilent Technologies Ultra Performance Liquid Chromatograph (1290 Infinity II), fitted with a single quadrupole mass spectrometer (Agilent Technologies 6130) and protein binding affinity in buffered aqueous solution.

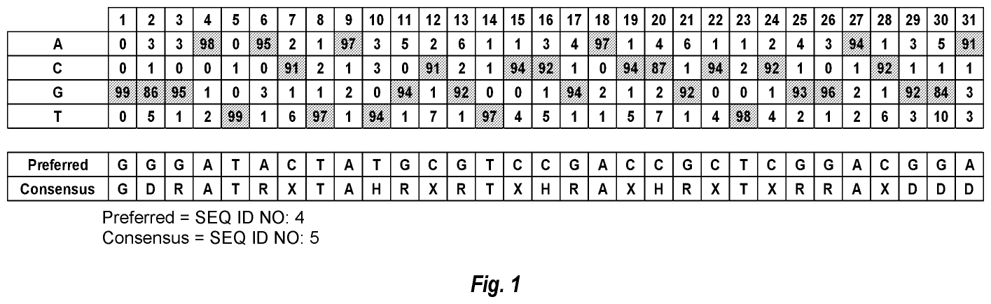

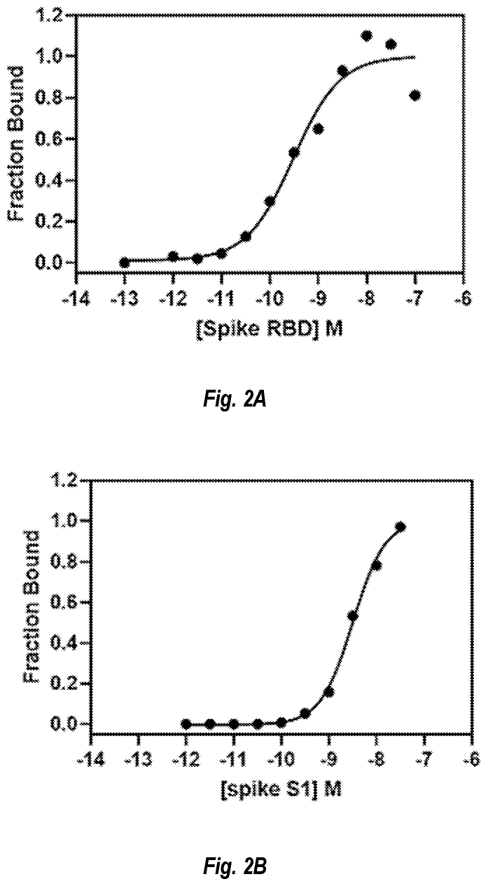

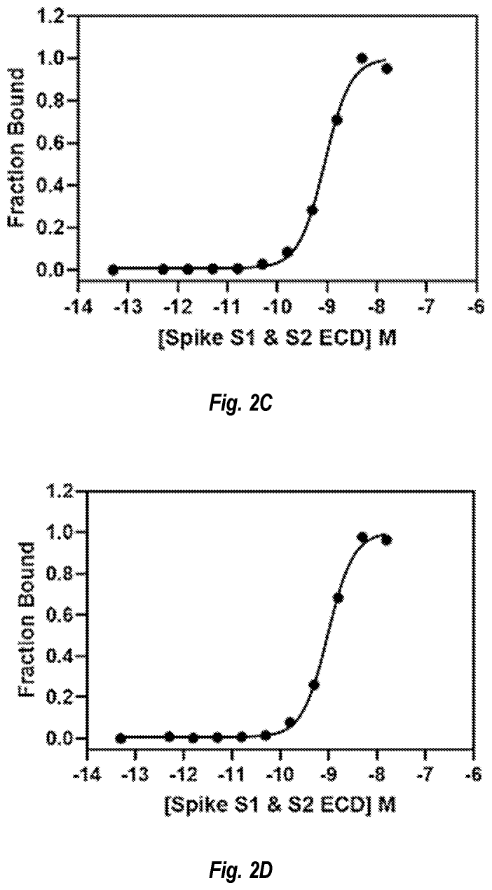

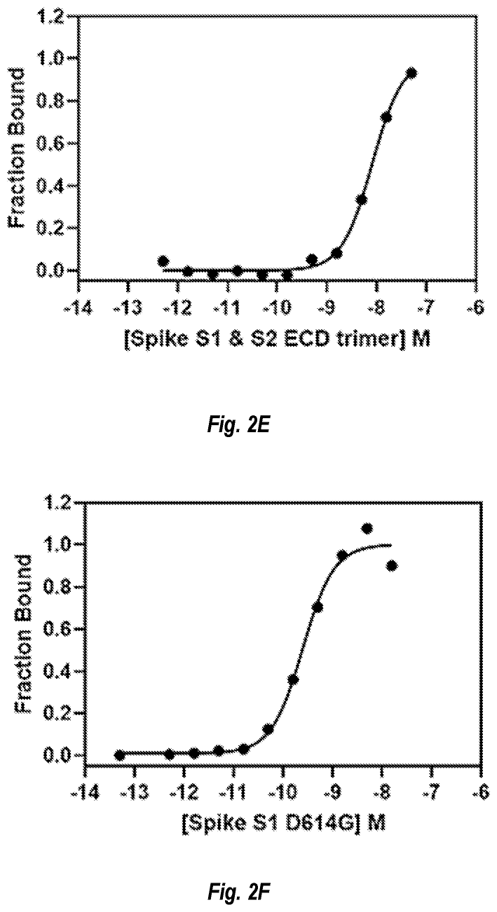

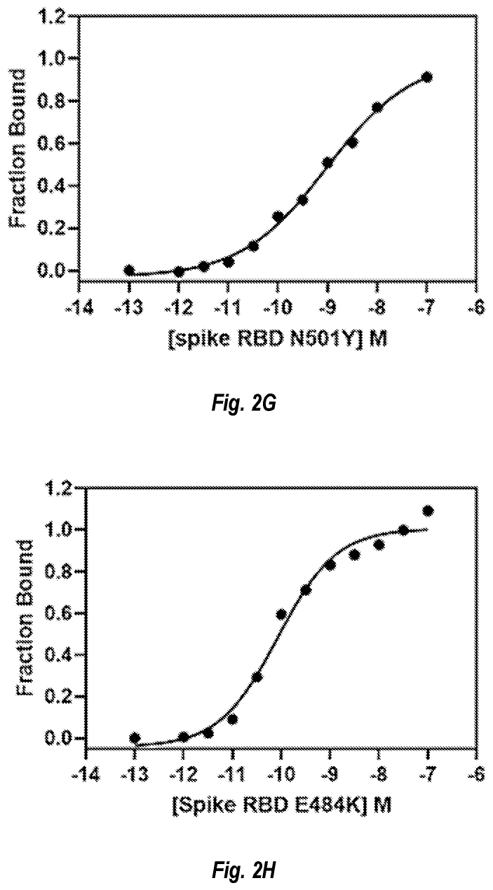

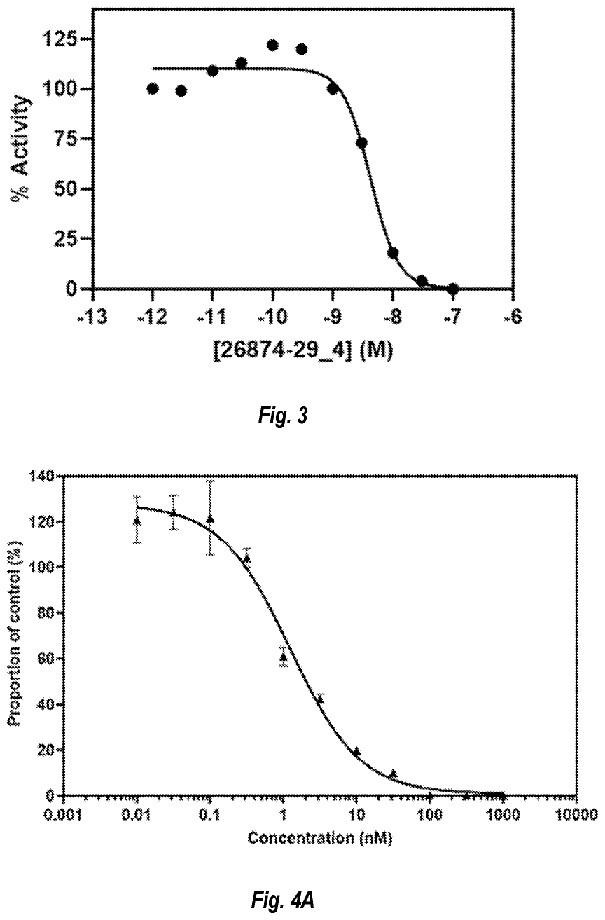

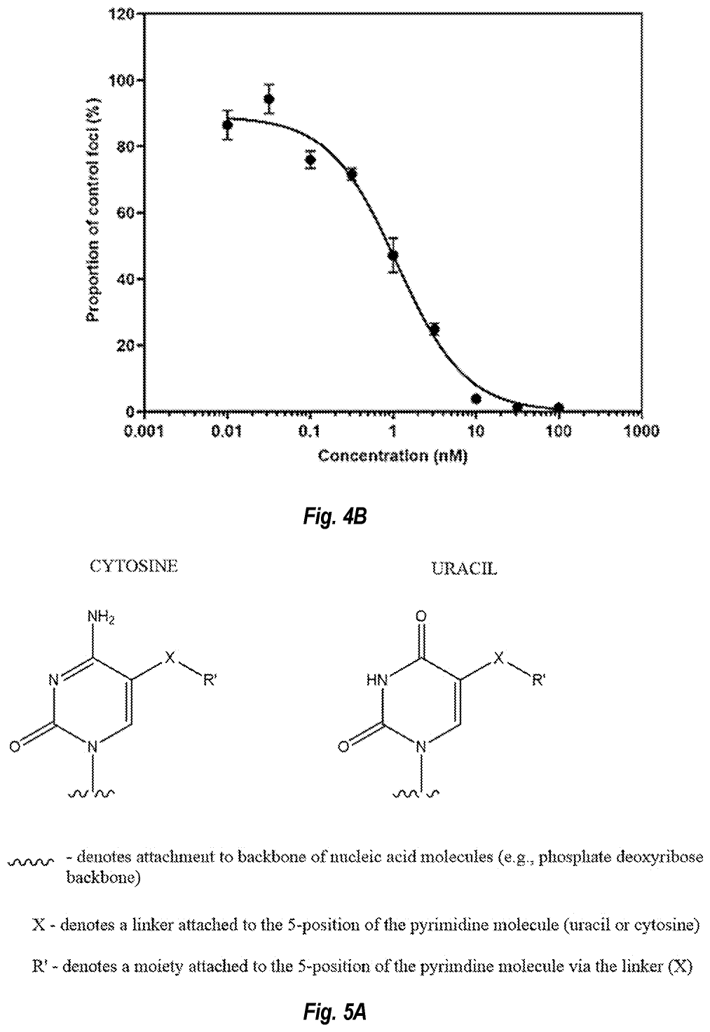

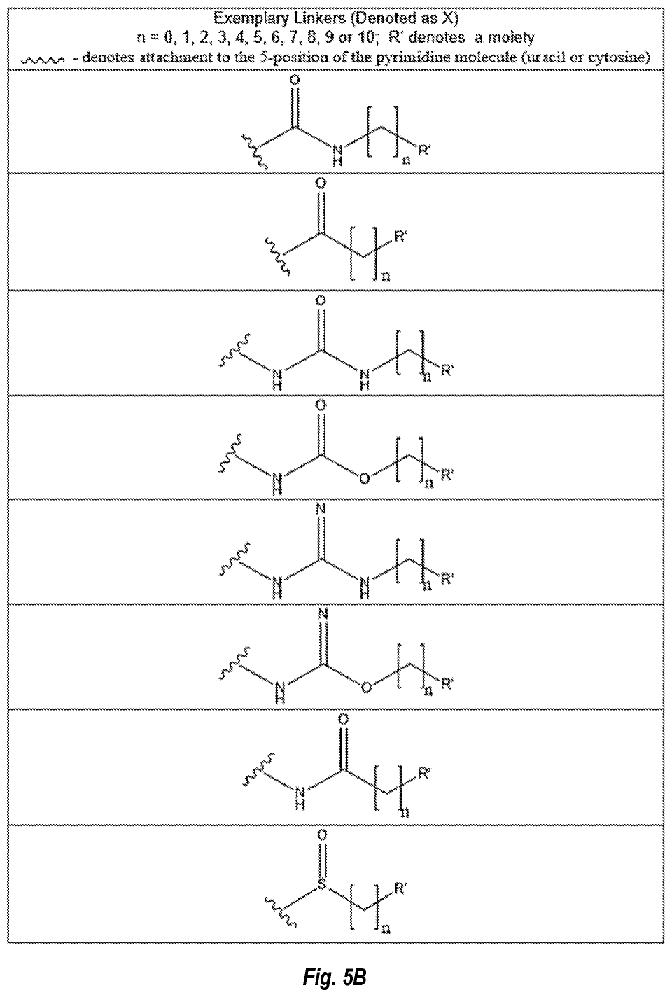

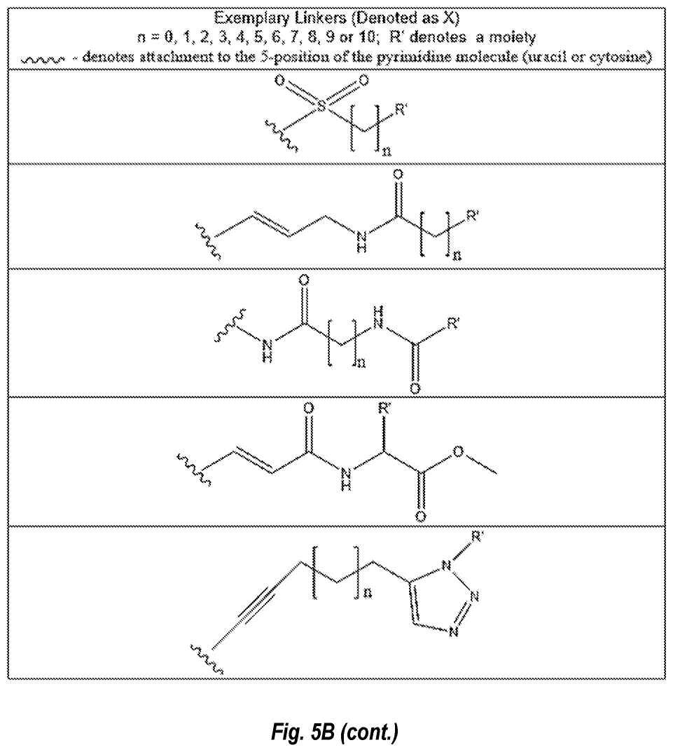

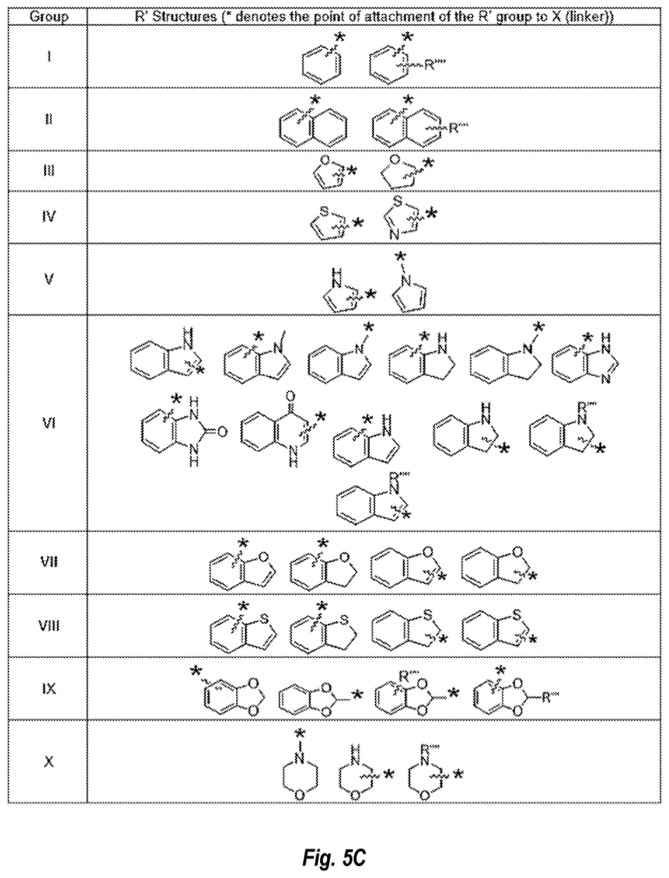

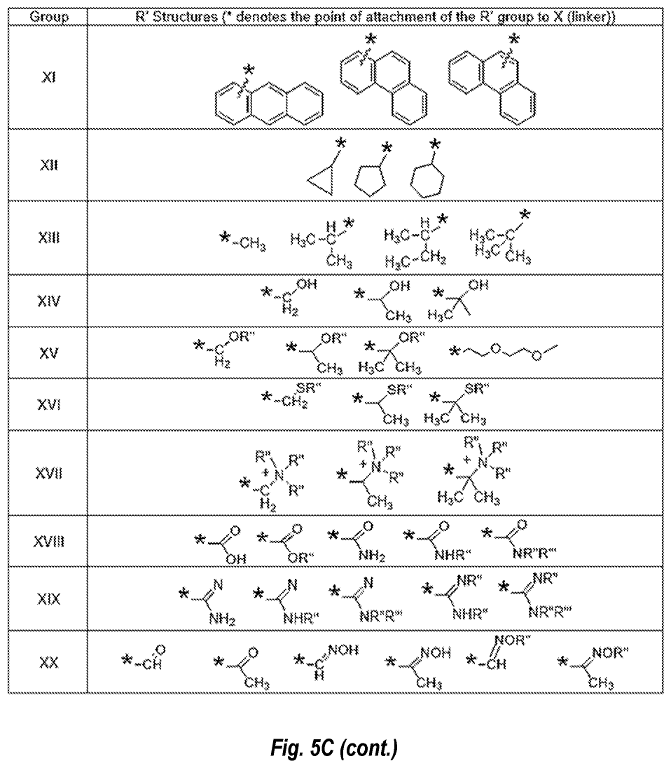

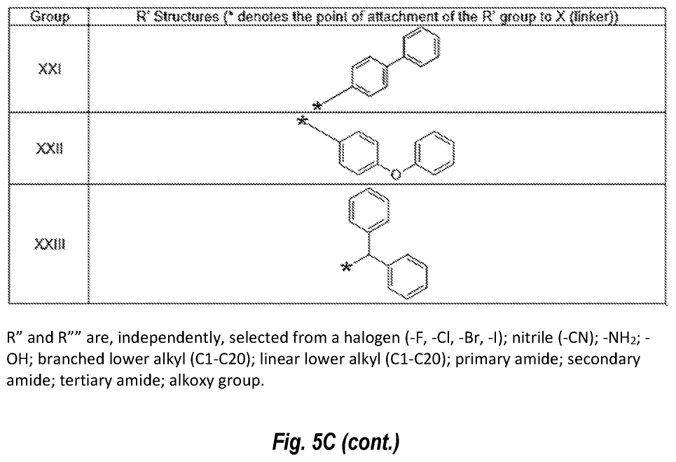

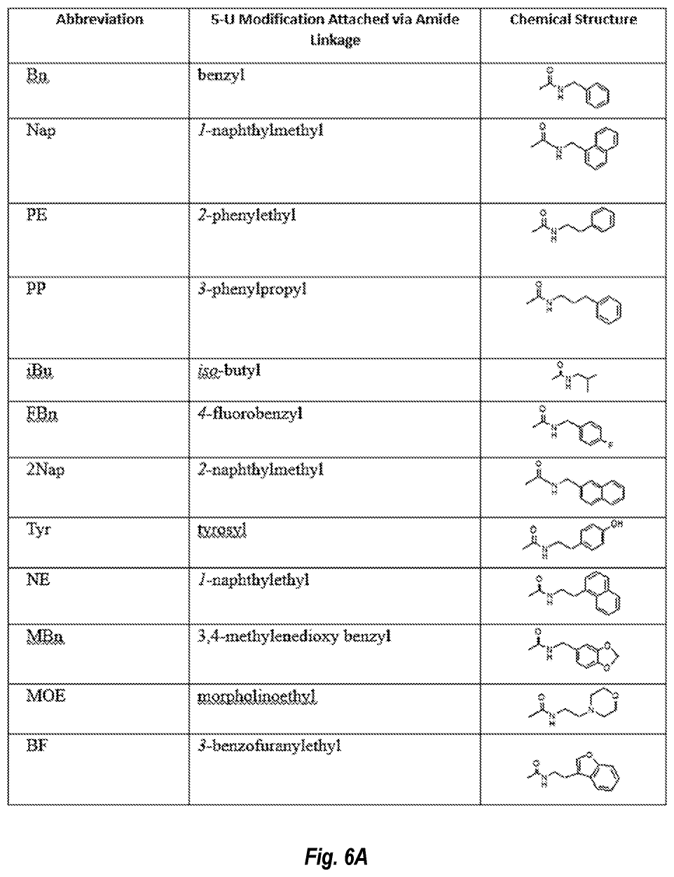

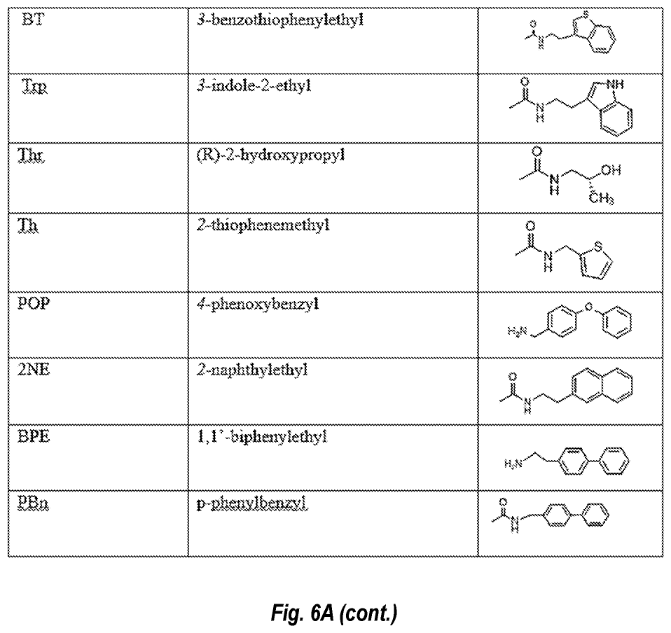

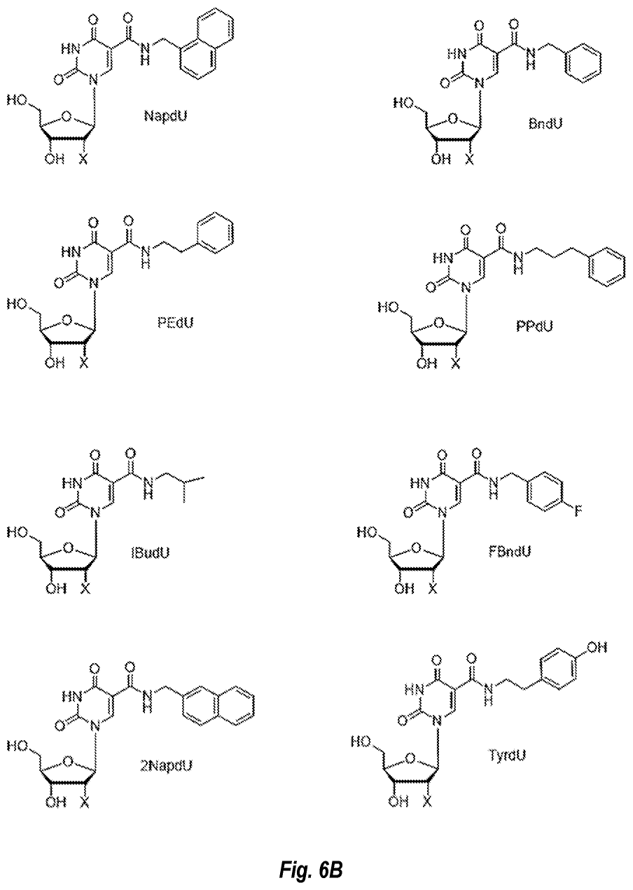

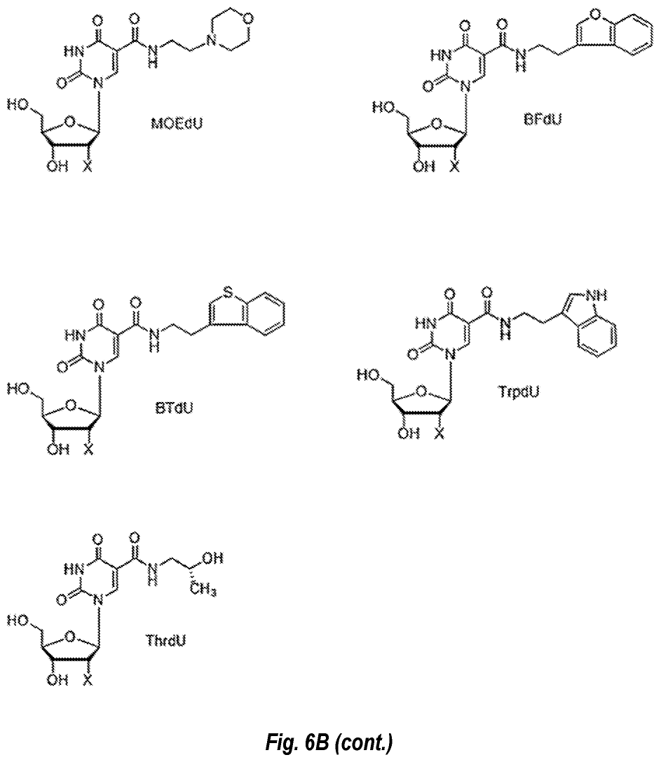

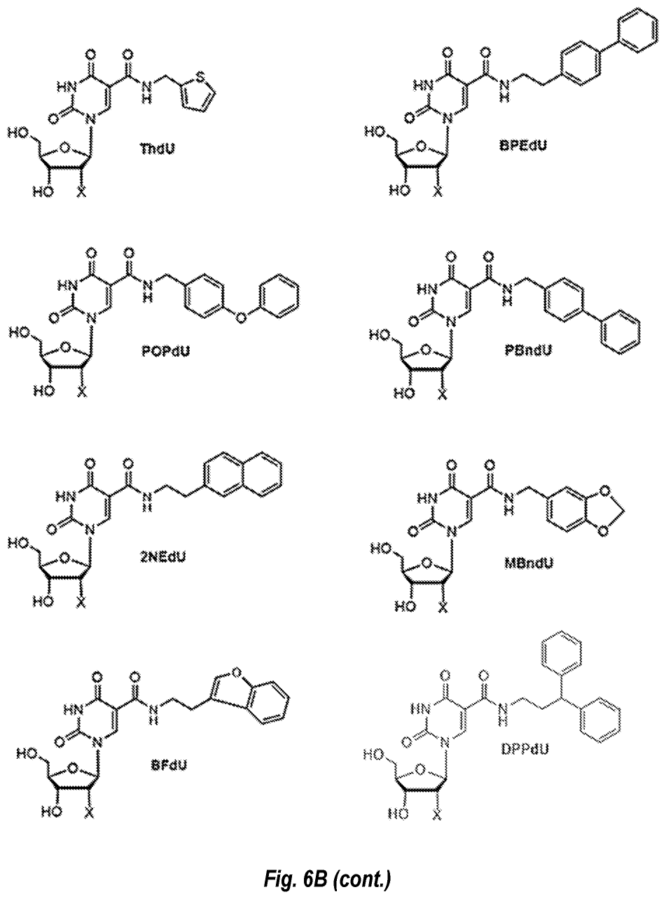

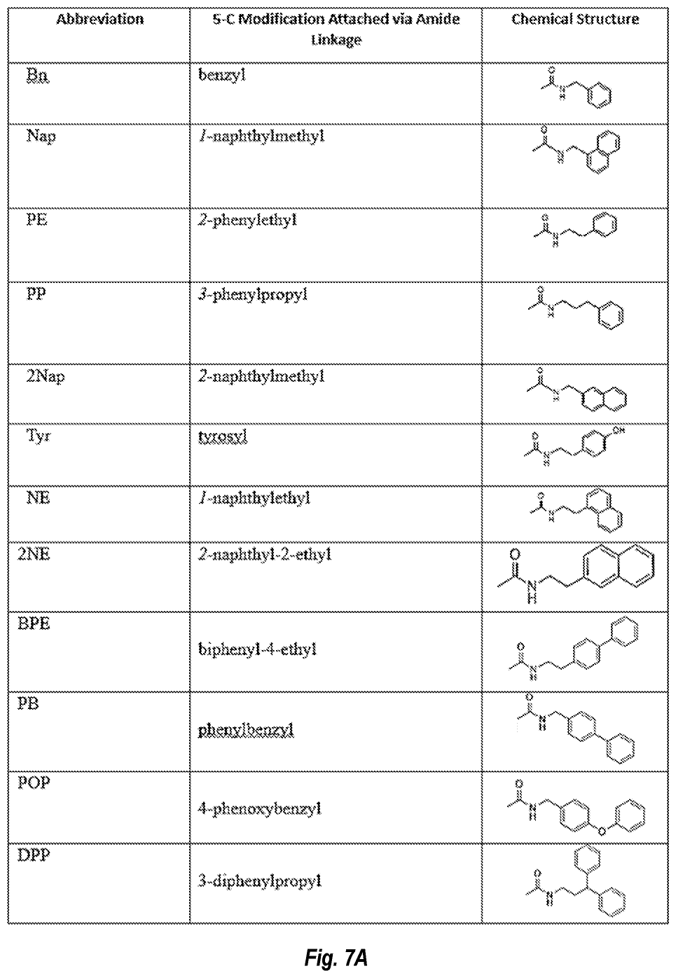

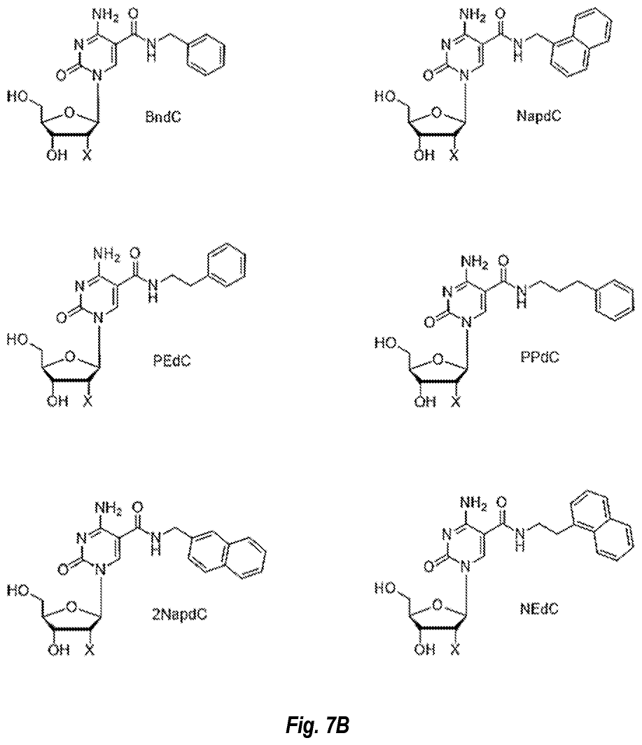

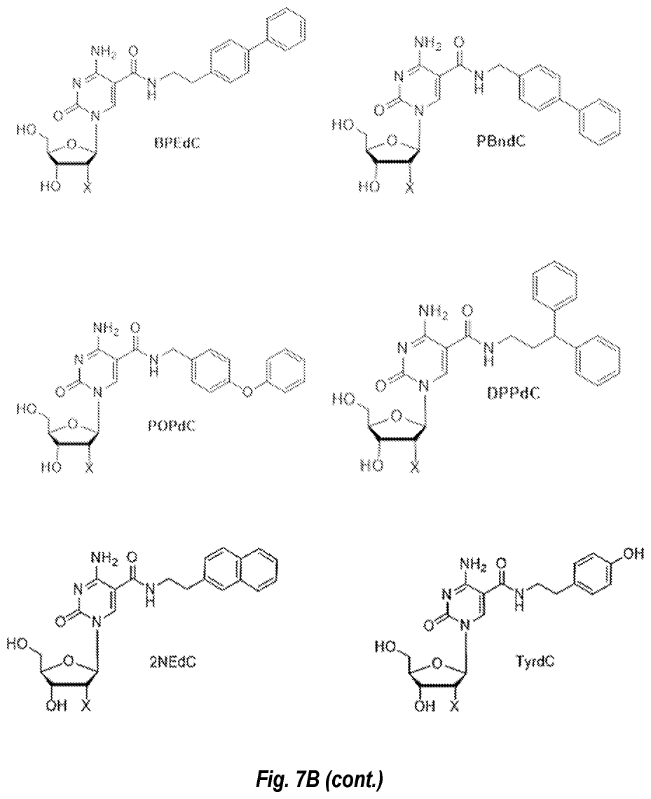

One of the sequences that was chosen for further characterization was 26874-29, and additional sequencing studies were conducted on the sequence pool from which this sequence was selected. 436 copies of this sequence, or closely related sequences, were identified from the sequence pool and used to define a preferred sequence and a consensus sequence for a spike aptamer. As illustrated in FIG. 1 the referred sequence is:

(SEQ ID NO: 4)
GGGATACTATGCGTCCGACCGCTCGGACGGA

and the consensus sequence is:

(SEQ ID NO: 5)
GDRATRXTAHRXRTXHRAXHRXTXRRAXDDD

wherein
A represents dA;
G represents dG;
C represents Nap-dC;
T represents Tyr-dU;
R is independently selected from a dA or dG;
X is independently selected from a Nap-dC or Tyr-dU;
H is independently selected from a dA, Nap-dC or Tyr-dU; and
D is independently selected from a dA, dG or Tyr-dU.

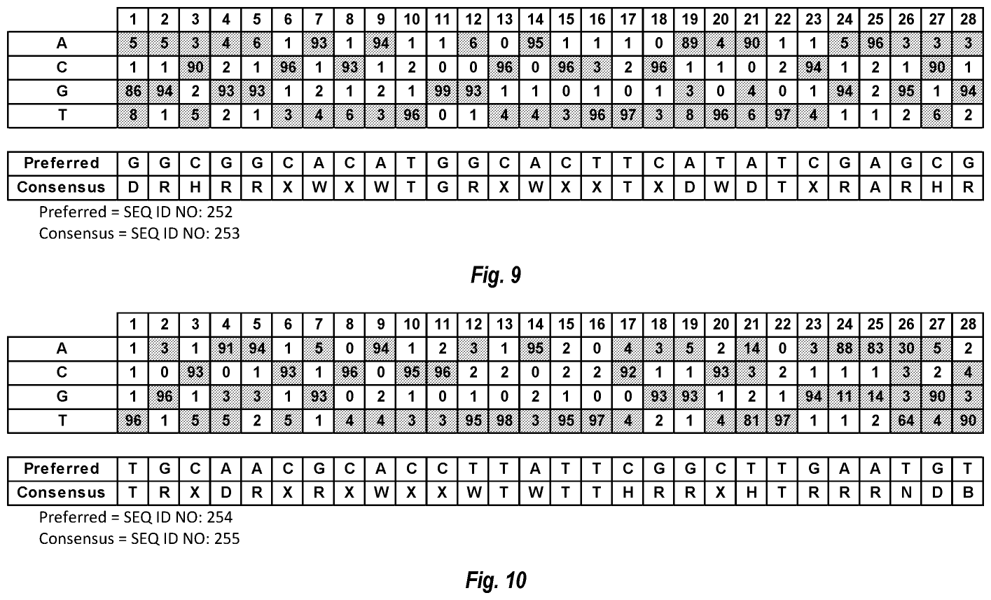

Another sequence that was chosen for further characterization was 26876-3, and additional sequencing studies were conducted on the sequence pool from which this sequence was selected. 191 copies of this sequence, or closely related sequences, were identified from a sequence pool and used to define a preferred sequence and a consensus sequence for a spike aptamer. A nucleotide frequency >2.5% was required at each position to define the consensus sequence. As illustrated in FIG. 9, the preferred sequence is:

(SEQ ID NO: 252)
GGCGGCACATGGCACTTCATATCGAGCG

and the consensus sequence is:

(SEQ ID NO: 253)
DRHRRXWXWTGRXWXXTXDWDTXRARHR

wherein
A represents dA;
G represents dG;
C represents Nap-dC;
T represents Tyr-dU;
R is independently selected from a dA or dG;
W is independently selected from dA or Tyr-dU;
X is independently selected from Nap-dC or Tyr-dU;
H is independently selected from dA, Nap-dC or Tyr-dU;
D is independently selected from dA, dG or Tyr-dU

Another sequence that was chosen for further characterization was 26876-13, and additional sequencing studies were conducted on the sequence pool from which this sequence was selected. 585 copies of this sequence, or closely related sequences, were identified from a sequence pool and used to define a preferred sequence and a consensus sequence for a spike aptamer. A nucleotide frequency >2.5% was required at each position to define the consensus sequence. As illustrated in FIG. 10, the preferred sequence is:

(SEQ ID NO: 254)
TGCAACGCACCTTATTCGGCTTGAATGT

and the consensus sequence is:

(SEQ ID NO: 255)
TRXDRXRXWXXWTWTTHRRXHTRRRNDB

wherein
A represents dA;
G represents dG;
C represents Nap-dC;
T represents Tyr-dU;
R is independently selected from a dA or dG;
W is independently selected from dA or Tyr-dU;
X is independently selected from Nap-dC or Tyr-dU;
H is independently selected from dA, Nap-dC or Tyr-dU;
D is independently selected from dA, dG or Tyr-dU;
B is independently selected from Nap-dC, dG or Tyr-dU;
N is independently selected from dA, Nap-dC, dG or Tyr-dU

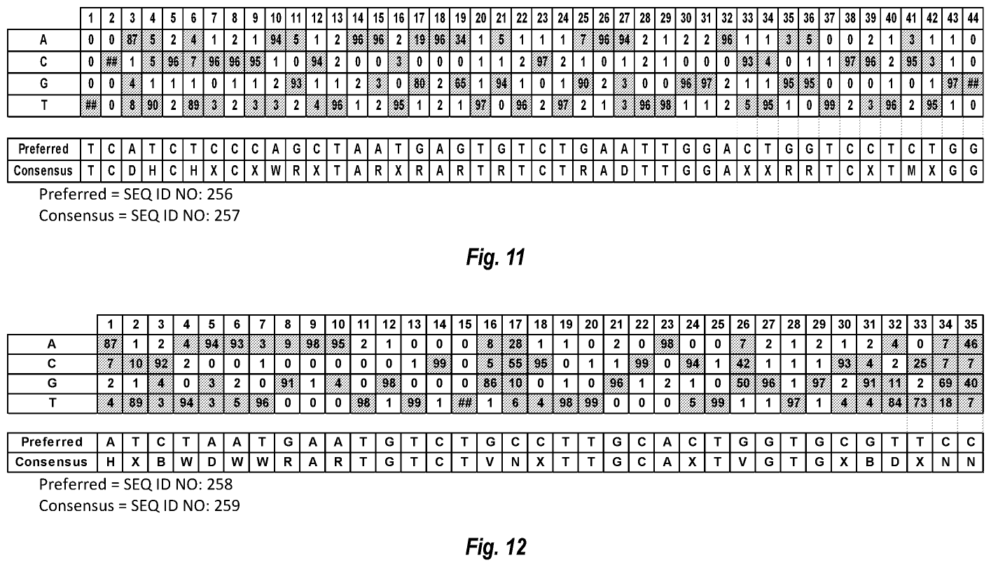

Another sequence that was chosen for further characterization was 26860-75, and additional sequencing studies were conducted on the sequence pool from which this sequence was selected. 920 copies of this sequence, or closely related sequences, were identified from a sequence pool and used to define a preferred sequence and a consensus sequence for a spike aptamer. A nucleotide frequency >2.5% was required at each position to define the consensus sequence. As illustrated in FIG. 11, the preferred sequence is:

(SEQ ID NO: 256)
TCATCTCCCAGCTAATGAGTGTCTGAATTGGACTGGTCCTCTGG

and the consensus sequence is:

(SEQ ID NO: 257)
TCDHCHXCXWRXTARXRARTRTCTRADTTGGAXXRRTCXTMXGG

wherein
A represents dA;
G represents dG;
C represents dC;
T represents Nap-dU;
R is independently selected from a dA or dG;
M is independently selected from a dA or dC;
W is independently selected from dA or Nap-dU;
X is independently selected from dC or Nap-dU;
H is independently selected from dA, dC or Nap-dU;
D is independently selected from dA, dG or Nap-dU

Another sequence that was chosen for further characterization was 26860-16, and additional sequencing studies were conducted on the sequence pool from which this sequence was selected. 255 copies of this sequence, or closely related sequences, were identified from a sequence pool and used to define a preferred sequence and a consensus sequence for a spike aptamer. A nucleotide frequency >2.5% was required at each position to define the consensus sequence. As illustrated in FIG. 12, the preferred sequence is:

(SEQ ID NO: 258)
ATCTAATGAATGTCTGCCTTGCACTGGTGCGTTCC

and the consensus sequence is:

(SEQ ID NO: 259)
HXBWDWWRARTGTCTVNXTTGCAXTVGTGXBDXNN

wherein
A represents dA;
G represents dG;
C represents dC;
T represents Nap-dU;
R is independently selected from a dA or dG;
M is independently selected from a dA or dC;
W is independently selected from dA or Nap-dU;
X is independently selected from dC or Nap-dU;
H is independently selected from dA, dC or Nap-dU;
D is independently selected from dA, dG or Nap-dU;
V is independently selected from dA, dC or dG;
B is independently selected from dC, dG or Nap-dU;
N is independently selected from dA, dC, dG or Nap-dU

### Example 3: Equilibrium Binding Constant (K_{d}) for Aptamers to Coronavirus Protein(s)

This example provides the method used herein to measure aptamer-coronavirus protein binding affinities and to determine K_{d}. Binding constants (K_{d} values) of modified aptamers were determined by filter binding assay for binding to SARS-CoV-2 spike protein, including the spike receptor binding domain (RBD), spike S1 & S2 extracellular domain (ECD), spike S1 domain, spike active trimer and spike proteins containing point mutations at specific amino acid residues. Spike proteins used for binding assays was purchased from commercial vendors: spike S1 and S2 ECD (SinoBiological, Cat #40589-V08B1), spike S1 Receptor Binding Domain (RBD) (Creative Biomart, Cat #Spike-190V), spike S1 subunit SARS-CoV-2 (Creative Biomart, Cat #Spike-191V), spike S1 and S2 stable trimer (Acro Biosystems, Cat #SPN-C52H9-50 ug), spike S1 aspartic acid 614 to glycine mutant (D614G) (Acro Biosystems, Cat #S1N-C5256-100 ug), spike RBD asparagine 501 to tyrosine mutant (N501Y) (Sino Biological, Cat #40592-V08H82), and spike RBD glutamic acid 484 to lysine mutant (E484K) (Acro Biosystems, Cat #SRD-C52H3-100 ug).

K_{d} values of modified aptamers were measured in 1×SB18T0.01. Modified aptamers were 5′ end labeled using T4 polynucleotide kinase (New England Biolabs) and γ7-[³²P]ATP (Perkin-Elmer). Radiolabeled aptamers (20,000 CPM, ˜0.03 nM) were mixed with spike proteins at concentrations ranging from or 10⁻⁷ or 10⁻⁸ to 10⁻¹² M and incubated at 37° C. for 45 minutes to 24 hours. Following incubation, reactions were mixed with an equal volume of 10 mM Dextran Sulfate and 0.014 mg of His-tag Dyna beads (Invitrogen) and incubated with mixing at 37° C. for 5 minutes. Bound complexes were captured on Durapore filter plates (EMD Millipore) and the fraction of bound aptamer was quantified with a phosphorimager (Typhoon FLA 9500, GE) and data were analyzed in ImageQuant (GE).

To determine binding affinity, data were fit using the equation:

y=(max−min)(Protein)/(K_{d}+Protein)+min. and plotted using GraphPad Prism version 7.00.

The 26874-29 sequence identified in the enriched pool was synthesized as a 40-nucleotide sequence, comprising the 30N modified nucleotide containing region and the last five nucleotides from primer 1 and the first five nucleotides from primer 2 sequences (Aptamer ID 26874-29_4). Using the filter binding assay, the affinity of sequence 26874-29_4 for spike RBD, spike S1 and spike S1 & S2 ECD was determined to be 3.0×10⁻¹⁰ M, 3.1×10⁻¹⁰ M and 7.3×10⁻¹⁰ M, respectively. Progressively shorter sequences of 26874-29_4 were then synthesized by systematically removing nucleotides from the 5′ and 3′ ends in order to identify the minimal sequence length required to maintain high affinity binding to spike protein (Aptamer ID 26874-29_6 through Aptamer ID 26874-29_25). These sequences were also evaluated in a filter binding assay and it was determined that Aptamer ID 26874-29_20, a 29-nucleotide sequence, was the minimal sequence required to maintain high affinity binding to spike protein. Sequences tested for binding are shown in Table 3 below and the binding affinities of aptamers are shown in Table 4 below.

Over the past year, several significant mutations to the spike protein have been found circulating in the general population. Mutations to the spike protein can affect the interaction with the ACE2 receptor, sometimes leading to more potent and/or contagious variants. Many of these mutated spike variants are available commercially as is the more physiologically relevant spike trimer protein. The 26874-29_20 sequence was therefore tested for maintaining binding to several mutated spike proteins and the spike trimer and found high affinity binding is upheld. Sequences tested for binding are shown in Table 3 and the binding affinities of aptamers are shown in Table 4 below. The amino acid sequences of the spike proteins used in the binding assays are shown in Table 5.

Having identified the shortest sequence as Aptamer ID 26874-29_20, which maintains the same binding affinity to spike protein as Aptamer ID 26874-29_4, additional modification substitutions were performed sequentially at all dU positions in the 29-mer sequence to identify reagents with higher affinity, using the filter binding assay described above. Twenty-two different modifications were substituted at the five dU positions in 26874-29_20. Several sequences showed approximately three-fold improvement in binding affinity to spike S1 & S2 ECD (26874-29_60, 26874-29_52, 26874-29_153, 26874-29_62, 26874-29_77, 26874-29_72, and 26874-29_90) compared to Aptamer ID 26874-29_20. The remaining sequences had approximately equal binding affinity as the parent 26874-29_20. Sequences tested in this optimization screen and the binding affinities to spike S1 & S2 ECD protein are shown in Tables 6A and 6B.

TABLE 3

| Aptamer ID | Sequence (5′ to 3′) | SEQ ID NO. |
|---|---|---|
| 26874-29_4 | CCTTGGGAYApYAYGpGYpp | 6 |
| | GAppGpYpGGApGGAGAAGG | |
| 26874-29_6 | TTGGGAYApYAYGpGYppGA | 8 |
| | ppGpYpGGApGGAGAAGG | |
| 26874-29_7 | GGGAYApYAYGpGYppGApp | 9 |
| | GpYpGGApGGAGAAGG | |
| 26874-29_12 | CCTTGGGAYApYAYGpGYpp | 10 |
| | GAppGpYpGGApGGAGAA | |
| 26874-29_13 | CCTTGGGAYApYAYGpGYpp | 11 |
| | GAppGpYpGGApGGAG | |
| 26874-29 14 | CCTTGGGAYApYAYGpGYpp | 12 |
| | GAppGpYpGGApGG | |
| 26874-29_15 | CCTTGGGAYAPYAYGpGYp | 13 |
| | pGAppGpYpGGAp | |
| 26874-29_18 | GGGAYApYAYGpGYppGAp | 14 |
| | pGpYpGGApGG | |
| 26874-29_19 | GGAYApYAYGpGYppGApp | 15 |
| | GpYpGGApGG | |
| 26874-29_20 | GGGAYApYAYGpGYppGAp | 7 |
| | pGpYpGGApG | |
| 26874-29_21 | GGAYApYAYGpGYppGApp | 16 |
| | GpYpGGApG | |
| 26874-29_22 | YApYAYGpGYppGAppGpY | 17 |
| | pGGApGG | |
| 26874-29_23 | ApYAYGpGYppGAppGpYp | 18 |
| | GGApGG | |
| 26874-29_24 | YApYAYGpGYppGAppGpY | 19 |
| | pGGApG | |
| 26874-29_25 | ApYAYGpGYppGAppGpY | 20 |
| | pGGApG | |

In Table 3, the following C-5 modified pyrimidines are shown: Y: 5-(p-hydroxyphenylethyl)-1-aminocarbonyl-2′-deoxyuridine (Tyr-dU); p: 5-(1-Naphthylmethyl)aminocarbonyl-2′-deoxycytidine (Nap-dC)

TABLE 4

| Aptamer ID | Protein | K_{d} (M) |
|---|---|---|
| 26874-29_4 | Spike RBD | 3.0E−10 |
| 26874-29_4 | Spike S1 | 3.2E−9 |
| 26874-29_4 | Spike S1 & S2 ECD | 7.3E−10 |
| 26874-29_20 | Spike S1 & S2 ECD | 9.6E−10 |
| 26874-29_20 | Spike S1 D614G | 2.5E−10 |
| 26874-29_20 | Spike RBD N501Y | 9.4E−10 |
| 26874-29_20 | Spike RBD E484K | 8.3E−11 |
| 26874-29_20 | Spike S1 & S2 ECD trimer | 8.1E−9 |
| 26874-29_6 | Spike S1 & S2 ECD | 7.9E−10 |
| 26874-29_7 | Spike S1 & S2 ECD | 8.9E−10 |
| 26874-29_12 | Spike S1 & S2 ECD | 6.0E−10 |
| 26874-29_13 | Spike S1 & S2 ECD | 7.2E−10 |
| 26874-29_14 | Spike S1 & S2 ECD | 6.9E−10 |
| 26874-29_15 | Spike S1 & S2 ECD | 1.8E−09 |
| 26874-29_18 | Spike S1 & S2 ECD | 9.8E−10 |
| 26874-29_19 | Spike S1 & S2 ECD | 1.5E−09 |
| 26874-29_20 | Spike S1 & S2 ECD | 6.9E−10 |
| 26874-29_21 | Spike S1 & S2 ECD | 2.2E−09 |
| 26874-29_22 | Spike S1 & S2 ECD | 1.4E−09 |
| 26874-29_23 | Spike S1 & S2 ECD | 1.6E−09 |
| 26874-29_24 | Spike S1 & S2 ECD | 2.1E−09 |
| 26874-29_25 | Spike S1 & S2 ECD | 1.8E−09 |

TABLE 5

| | | SEQ |
|---|---|---|
| | | ID |
| Protein | Sequence | NO. |
| Spike RBD | RVQPTESIVRFPNITNLCPFGEVFNATRFA | 21 |
| | SVYAWNRKRISNCVADYSVLYNSASFSTFK | |
| | CYGVSPTKLNDLCFTNVYADSFVIRGDEVR | |
| | QIAPGQTGKIADYNYKLPDDFTGCVIAWNS | |
| | NNLDSKVGGNYNYLYRLFRKSNLKPFERDI | |
| | STEIYQAGSTPCNGVEGFNCYFPLQSYGFQ | |
| | PTNGVGYQPYRVVVLSFELLHAPATVCGPK | |
| | KSTNLVKNKCVNF | |
| Spike S1 | VNLTTRTQLPPAYTNSFTRGVYYPDKVFRS | 22 |
| | SVLHSTQDLFLPFFSNVTWFHAIHVSGTNG | |
| | TKRFDNPVLPFNDGVYFASTEKSNIIRGWI | |
| | FGTTLDSKTQSLLIVNNATNVVIKVCEFQF | |
| | CNDPFLGVYYHKNNKSWMESEFRVYSSANN | |
| | CTFEYVSQPFLMDLEGKQGNFKNLREFVFK | |
| | NIDGYFKIYSKHTPINLVRDLPQGFSALEP | |
| | LVDLPIGINITRFQTLLALHRSYLTPGDSS | |
| | SGWTAGAAAYYVGYLQPRTFLLKYNENGTI | |
| | TDAVDCALDPLSETKCTLKSFTVEKGIYQT | |
| | SNFRVQPTESIVRFPNITNLCPFGEVFNAT | |
| | RFASVYAWNRKRISNCVADYSVLYNSASFS | |
| | TFKCYGVSPTKLNDLCFTNVYADSFVIRGD | |
| | EVRQIAPGQTGKIADYNYKLPDDFTGCVIA | |
| | WNSNNLDSKVGGNYNYLYRLFRKSNLKPFE | |
| | RDISTEIYQAGSTPCNGVEGFNCYFPLQSY | |
| | GFQPTNGVGYQPYRVVVLSFELLHAPATVC | |
| | GPKKSTNLVKNKCVNFNFNGLTGTGVLTES | |
| | NKKFLPFQQFGRDIADTTDAVRDPQTLEIL | |
| | DITPCSFGGVSVITPGTNTSNQVAVLYQDV | |
| | NCTEVPVAIHADQLTPTWRVYSTGSNVFQT | |
| | RAGCLIGAEHVNNSYECDIPIGAGICASYQ | |
| | TQTNSP | |
| Spike S1 & | VNLTTRTQLPPAYTNSFTRGVYYPDKVFRS | 23 |
| S2 ECD | SVLHSTQDLFLPFFSNVTWFHAIHVSGTNG | |
| | TKRFDNPVLPFNDGVYFASTEKSNIIRGWI | |
| | FGTTLDSKTQSLLIVNNATNVVIKVCEFQF | |
| | CNDPFLGVYYHKNNKSWMESEFRVYSSANN | |
| | CTFEYVSQPFLMDLEGKQGNFKNLREFVFK | |
| | NIDGYFKIYSKHTPINLVRDLPQGFSALEP | |
| | LVDLPIGINITRFQTLLALHRSYLTPGDSS | |
| | SGWTAGAAAYYVGYLQPRTFLLKYNENGTI | |
| | TDAVDCALDPLSETKCTLKSFTVEKGIYQT | |
| | SNFRVQPTESIVRFPNITNLCPFGEVFNAT | |
| | RFASVYAWNRKRISNCVADYSVLYNSASFS | |
| | TFKCYGVSPTKLNDLCFTNVYADSFVIRGD | |
| | EVRQIAPGQTGKIADYNYKLPDDFTGCVIA | |
| | WNSNNLDSKVGGNYNYLYRLFRKSNLKPFE | |
| | RDISTEIYQAGSTPCNGVEGFNCYFPLQSY | |
| | GFQPTNGVGYQPYRVVVLSFELLHAPATVC | |
| | GPKKSTNLVKNKCVNFNFNGLTGTGVLTES | |
| | NKKFLPFQQFGRDIADTTDAVRDPQTLEIL | |
| | DITPCSFGGVSVITPGTNTSNQVAVLYQDV | |
| | NCTEVPVAIHADQLTPTWRVYSTGSNVFQT | |
| | RAGCLIGAEHVNNSYECDIPIGAGICASYQ | |
| | TQTNSPRRARSVASQSHIAYTMSLGAENSV | |
| | AYSNNSIAIPTNFTISVTTEILPVSMTKTS | |
| | VDCTMYICGDSTECSNLLLQYGSFCTQLNR | |
| | ALTGIAVEQDKNTQEVFAQVKQIYKTPPIK | |
| | DFGGFNFSQILPDPSKPSKRSFIEDLLFNK | |
| | VTLDAGFIKQYGDCLGDIAARDLICAQKFN | |
| | GLTVLPPLLTDEMIAQYTSALLAGTITSGW | |
| | TFGAGAALQIPFAMQMAYRENGIGVTQNVL | |
| | YENQKLIANQFNSAIGKIQDSLSSTASALG | |
| | KLQDVVNQNAQALNTLVKQLSSNFGAISSV | |
| | LNDILSRLDKVEAEVQIDRLITGRLQSLQT | |
| | YVTQQLIRAAEIRASANLAATKMSECVLGQ | |
| | SKRVDFCGKGYHLMSFPQSAPHGVVFLHVT | |
| | YVPAQEKNFTTAPAICHDGKAHFPREGVFV | |
| | SNGTHWFVTQRNFYEPQIITTDNTFVSGNC | |
| | DVVIGIVNNTVYDPLQPELDSFKEELDKYF | |
| | KNHTSPDVDLGDISGINASVVNIQKEIDRL | |
| | NEVAKNLNESLIDLQELGKYEQYIKWP | |
| Spike S1 & | VNLTTRTQLPPAYTNSFTRGVYYPDKVFRS | 24 |
| S2 ECD | SVLHSTQDLFLPFFSNVTWFHAIHVSGTNG | |
| stable | TKRFDNPVLPFNDGVYFASTEKSNIIRGWI | |
| trimer | FGTTLDSKTQSLLIVNNATNVVIKVCEFQF | |
| | CNDPFLGVYYHKNNKSWMESEFRVYSSANN | |
| | CTFEYVSQPFLMDLEGKQGNFKNLREFVFK | |
| | NIDGYFKIYSKHTPINLVRDLPQGFSALEP | |
| | LVDLPIGINITRFQTLLALHRSYLTPGDSS | |
| | SGWTAGAAAYYVGYLQPRTFLLKYNENGTI | |
| | TDAVDCALDPLSETKCTLKSFTVEKGIYQT | |
| | SNFRVQPTESIVRFPNITNLCPFGEVFNAT | |
| | RFASVYAWNRKRISNCVADYSVLYNSASFS | |
| | TFKCYGVSPTKLNDLCFTNVYADSFVIRGD | |
| | EVRQIAPGQTGKIADYNYKLPDDFTGCVIA | |
| | WNSNNLDSKVGGNYNYLYRLFRKSNLKPFE | |
| | RDISTEIYQAGSTPCNGVEGFNCYFPLQSY | |
| | GFQPTNGVGYQPYRVVVLSFELLHAPATVC | |
| | GPKKSTNLVKNKCVNFNFNGLTGTGVLTES | |
| | NKKFLPFQQFGRDIADTTDAVRDPQTLEIL | |
| | DITPCSFGGVSVITPGTNTSNQVAVLYQDV | |
| | NCTEVPVAIHADQLTPTWRVYSTGSNVFQT | |
| | RAGCLIGAEHVNNSYECDIPIGAGICASYQ | |
| | TQTNSPRAAASVASQSIIAYTMSLGAENSV | |
| | AYSNNSIAIPTNFTISVTTEILPVSMTKTS | |
| | VDCTMYICGDSTECSNLLLQYGSFCTQLNR | |
| | ALTGIAVEQDKNTQEVFAQVKQIYKTPPIK | |
| | DFGGFNFSQILPDPSKPSKRSPIEDLLFNK | |
| | VTLADAGFIKQYGDCLGDIAARDLICAQKF | |
| | NGLTVLPPLLTDEMIAQYTSALLAGTITSG | |
| | WTFGAGPALQIPFPMQMAYRFNGIGVTQNV | |
| | LYENQKLIANQFNSAIGKIQDSLSSTPSAL | |
| | GKLQDVVNQNAQALNTLVKQLSSNFGAISS | |
| | VLNDILSRLDPPEAEVQIDRLITGRLQSLQ | |
| | TYVTQQLIRAAEIRASANLAATKMSECVLG | |
| | QSKRVDFCGKGYHLMSFPQSAPHGVVFLHV | |
| | TYVPAQEKNFTTAPAICHDGKAHFPREGVF | |
| | VSNGTHWFVTQRNFYEPQIITTDNTFVSGN | |
| | CDVVIGIVNNTVYDPLQPELDSFKE | |
| Spike S1 | VNLTTRTQLPPAYTNSFTRGVYYPDKVFRS | 25 |
| D614G | SVLHSTQDLFLPFFSNVTWFHAIHVSGTNG | |
| | TKRFDNPVLPFNDGVYFASTEKSNIIRGWI | |
| | FGTTLDSKTQSLLIVNNATNVVIKVCEFQF | |
| | CNDPFLGVYYHKNNKSWMESEFRVYSSNNC | |
| | TFEYVSQPFLMDLEGKQGNFKNLREFVFKN | |
| | IDGYFKIYSKHTPINLVRDLPQGFSALEPL | |
| | VDLPIGINITRFQTLLALHRSYLTPGDSSS | |
| | GWTAGAAAYYVGYLQPRTFLLKYNENGTIT | |
| | DAVDCALDPLSETKCTLKSFTVEKGIYQTS | |
| | NFRVQPTESIVRFPNITNLCPFGEVFNATR | |
| | FASVYAWNRKRISNCVADYSVLYNSASFST | |
| | FKCYGVSPTKLNDLCFTNVYADSFVIRGDE | |
| | VRQIAPGQTGKIADYNYKLPDDFTGCVIAW | |
| | NSNNLDSKVGGNYNYLYRLFRKSNLKPFER | |
| | DISTEIYQAGSTPCNGVEGFNCYFPLQSYG | |
| | FQPTNGVGYQPYRVVVLSFELLHAPATVCG | |
| | PKKSTNLVKNKCVNFNFNGLTGTGVLTESN | |
| | KKFLPFQQFGRDIADTTDAVRDPQTLEILD | |
| | ITPCSFGGVSVITPGTNTSNQVAVLYQGVN | |
| | CTEVPVAIHADQLTPTWRVYSTGSNVFQTR | |
| | AGCLIGAEHVNNSYECDIPIGAGICASYQT | |
| | QTNSPRRAR | |
| Spike RBD | RVQPTESIVRFPNITNLCPFGEVFNATRFA | 26 |
| N501Y | SVYAWNRKRISNCVADYSVLYNSASFSTFK | |
| | CYGVSPTKLNDLCFTNVYADSFVIRGDEVR | |
| | QIAPGQTGKIADYNYKLPDDFTGCVIAWNS | |
| | NNLDSKVGGNYNYLYRLFRKSNLKPFERDI | |
| | STEIYQAGSTPCNGVEGENCYFPLQSYGFQ | |
| | PTYGVGYQPYRVVVLSFELLHAPATVCGPK | |
| | KSTNLVKNKCVNF | |
| Spike RBD | RVQPTESIVRFPNITNLCPFGEVFNATRFA | 27 |
| E484K | SVYAWNRKRISNCVADYSVLYNSASFSTFK | |
| | CYGVSPTKLNDLCFTNVYADSFVIRGDEVR | |
| | QIAPGQTGKIADYNYKLPDDFTGCVIAWNS | |
| | NNLDSKVGGNYNYLYRLFRKSNLKPFERDI | |
| | STEIYQAGSTPCNGVKGFNCYFPLQSYGFQ | |
| | PTNGVGYQPYRVVVLSFELLHAPATVCGPK | |
| | KSTNLVKNK | |
| Spike S1 | MFVFLVLLPLVSSQCVNLTTRTQLPPAYTN | 123 |
| & S2 | SFTRGVYYPDKVFRSSVLHSTQDLFLPFFS | |
| ECD | NVTWFHAISGTNGTKRFDNPVLPENDGVYF | |
| B1.1.7 | ASTEKSNIIRGWIFGTTLDSKTQSLLIVNN | |
| variant | ATNVVIKVCEFQFCNDPFLGVYHKNNKSWM | |
| (Alpha) | ESEFRVYSSANNCTFEYVSQPFLMDLEGKQ | |
| | GNFKNLREFVFKNIDGYFKIYSKHTPINLV | |
| | RDLPQGFSALEPLVDLPIGINITRFQTLLA | |
| | LHRSYLTPGDSSSGWTAGAAAYYVGYLQPR | |
| | TFLLKYNENGTITDAVDCALDPLSETKCTL | |
| | KSFTVEKGIYQTSNERVQPTESIVRFPNIT | |
| | NLCPFGEVENATRFASVYAWNRKRISNCVA | |
| | DYSVLYNSASFSTFKCYGVSPTKLNDLCFT | |
| | NVYADSFVIRGDEVRQIAPGQTGKIADYNY | |
| | KLPDDFTGCVIAWNSNNLDSKVGGNYNYLY | |
| | RLERKSNLKPFERDISTEIYQAGSTPCNGV | |
| | KGFNCYFPLQPYGFQPTYGVGYQPYRVVVL | |
| | SFELLHAPATVCGPKKSTNLVKNKCVNFNF | |
| | NGLTGTGVLTESNKKFLPFQQFGRDIDDTT | |
| | DAVRDPQTLEILDITPCSFGGVSVITPGTN | |
| | TSNQVAVLYQGVNCTEVPVAIHADQLTPTW | |
| | RVYSTGSNVFQTRAGCLIGAEHVNNSYECD | |
| | IPIGAGICASYQTQTNSHRRARSVASQSII | |
| | AYTMSLGAENSVAYSNNSIAIPINFTISVT | |
| | TEILPVSMTKTSVDCTMYICGDSTECSNLL | |
| | LQYGSFCTQLNRALTGIAVEQDKNTQEVFA | |
| | QVKQIYKTPPIKDFGGFNFSQILPDPSKPS | |
| | KRSFIEDLLENKVTLADAGFIKQYGDCLGD | |
| | IAARDLICAQKFNGLTVLPPLLTDEMIAQY | |
| | TSALLAGTITSGWTFGAGAALQIPFAMQMA | |
| | YRFNGIGVTQNVLYENQKLIANQFNSAIGK | |
| | IQDSLSSTASALGKLQDVVNQNAQALNTLV | |
| | KQLSSNFGAISSVLNDILARLDKVEAEVQI | |
| | DRLITGRLQSLQTYVTQQLIRAAEIRASAN | |
| | LAATKMSECVLGQSKRVDFCGKGYHLMSFP | |
| | QSAPHGVVFLHVTYVPAQEKNFTTAPAICH | |
| | DGKAHFPREGVFVSNGTHWFVTQRNFYEPQ | |
| | IITTHNTFVSGNCDVVIGIVNNTVYDPLQP | |
| | ELDSFKEELDKYFKNHTSPDVDLGDISGIN | |
| | ASVVNIQKEIDRLNEVANNLNESLIDLQEL | |
| | GKYEQYIKWPWYIWLGFIAGLIAIVMVTIM | |
| | LCCMTSCCSCLKGCCSCGSCCKFDEDDSEP | |
| | VLKGVKLHYT | |
| Spike S1 | MFVFLVLLPLVSSQCVNLTTRTQLPPAYTN | 124 |
| & S2 | SFTRGVYYPDKVFRSSVLHSTQDLFLPFFS | |
| ECD | NVTWFHAIHVSGTNGTKRFANPVLPFNDGV | |
| B1.351 | YFASTEKSNIIRGWIFGTTLDSKTQSLLIV | |
| variant | NNATNVVIKVCEFQFCNDPFLGVYYHKNNK | |
| (Beta) | SWMESEFRVYSSANNCTFEYVSQPFLMDLE | |
| | GKQGNFKNLREFVFKNIDGYFKIYSKHTPI | |
| | NLVRGLPQGFSALEPLVDLPIGINITRFQT | |
| | LHRSYLTPGDSSSGWTAGAAAYYVGYLQPR | |
| | TELLKYNENGTITDAVDCALDPLSETKCTL | |
| | KSFTVEKGIYQTSNERVQPTESIVRFPNIT | |
| | NLCPFGEVENATRFASVYAWNRKRISNCVA | |
| | DYSVLYNSASFSTFKCYGVSPTKLNDLCFT | |
| | NVYADSFVIRGDEVRQIAPGQTGNIADYNY | |
| | KLPDDFTGCVIAWNSNNLDSKVGGNYNYLY | |
| | RLFRKSNLKPFERDISTEIYQAGSTPCNGV | |
| | KGFNCYFPLQSYGFQPTYGVGYQPYRVVVL | |
| | SFELLHAPATVCGPKKSTNLVKNKCVNFNF | |
| | NGLTGTGVLTESNKKELPFQQFGRDIADTT | |
| | DAVRDPQTLEILDITPCSFGGVSVITPGTN | |
| | TSNQVAVLYQGVNCTEVPVAIHADQLTPTW | |
| | RVYSTGSNVFQTRAGCLIGAEHVNNSYECD | |
| | IPIGAGICASYQTQTNSPRRARSVASQSII | |
| | AYTMSLGVENSVAYSNNSIAIPTNFTISVT | |
| | TEILPVSMTKTSVDCTMYICGDSTECSNLL | |
| | LQYGSFCTQLNRALTGIAVEQDKNTQEVFA | |
| | QVKQIYKTPPIKDFGGFNFSQILPDPSKPS | |
| | KRSFIEDLLENKVTLADAGFIKQYGDCLGD | |
| | IAARDLICAQKFNGLTVLPPLLTDEMIAQY | |
| | TSALLAGTITSGWTFGAGAALQIPFAMQMA | |
| | YRENGIGVTQNVLYENQKLIANQFNSAIGK | |
| | IQDSLSSTASALGKLQDVVNQNAQALNTLV | |
| | KQLSSNFGAISSVLNDILSRLDKVEAEVQI | |
| | DRLITGRLQSLQTYVTQQLIRAAEIRASAN | |
| | LAATKMSECVLGQSKRVDFCGKGYHLMSFP | |
| | QSAPHGVVFLHVTYVPAQEKNFTTAPAICH | |
| | DGKAHFPREGVFVSNGTHWFVTQRNFYEPQ | |
| | IITTDNTFVSGNCDVVIGIVNNTVYDPLQP | |
| | ELDSFKEELDKYFKNHTSPDVDLGDISGIN | |
| | ASVVNIQKEIDRLNEVAKNLNESLIDLQEL | |
| | GKYEQYIKWPWYIWLGFIAGLIAIVMVTIM | |
| | LCCMTSCCSCLKGCCSCGSCCKFDEDDSEP | |
| | VLKGVKLHYT | |
| Spike S1 | MFVFLVLLPLVSSQCVNFTNRTQLPSAYTN | 125 |
| & S2 | SFTRGVYYPDKVFRSSVLHSTQDLFLPFFS | |
| ECD P.1 | NVTWFHAIHVSGTNGTKRFDNPVLPFNDGV | |
| variant | YFASTEKSNIIRGWIFGTTLDSKTQSLLIV | |
| (Gamma) | NNATNVVIKVCEFQFCNYPFLGVYYHKNNK | |
| | SWMESEFRVYSSANNCTFEYVSQPFLMDLE | |
| | GKQGNFKNLSEFVFKNIDGYFKIYSKHTPI | |
| | NLVRDLPQGFSALEPLVDLPIGINITRFQT | |
| | LLALHRSYLTPGDSSSGWTAGAAAYYVGYL | |
| | QPRTFLLKYNENGTITDAVDCALDPLSETK | |
| | CTLKSFTVEKGIYQTSNERVQPTESIVRFP | |
| | NITNLCPFGEVENATRFASVYAWNRKRISN | |
| | CVADYSVLYNSASFSTFKCYGVSPTKLNDL | |
| | CFTNVYADSFVIRGDEVRQIAPGQTGTIAD | |
| | YNYKLPDDFTGCVIAWNSNNLDSKVGGNYN | |
| | YLYRLERKSNLKPFERDISTEIYQAGSTPC | |
| | NGVKGFNCYFPLQSYGFQPTYGVGYQPYRV | |
| | VVLSFELLHAPATVCGPKKSTNLVKNKCVN | |
| | FNFNGLTGTGVLTESNKKFLPFQQFGRDIA | |
| | DTTDAVRDPQTLEILDITPCSFGGVSVITP | |
| | GTNTSNQVAVLYQGVNCTEVPVAIHADQLT | |
| | PTWRVYSTGSNVFQTRAGCLIGAEYVNNSY | |
| | ECDIPIGAGICASYQTQTNSPRRARSVASQ | |
| | SIIAYTMSLGAENSVAYSNNSIAIPTNFTI | |
| | SVTTEILPVSMTKTSVDCTMYICGDSTECS | |
| | NLLLQYGSFCTQLNRALTGIAVEQDKNTQE | |
| | VFAQVKQIYKTPPIKDFGGFNFSQILPDPS | |
| | KPSKRSFIEDLLENKVTLADAGFIKQYGDC | |
| | LGDIAARDLICAQKFNGLTVLPPLLTDEMI | |
| | AQYTSALLAGTITSGWTFGAGAALQIPFAM | |
| | QMAYRENGIGVTQNVLYENQKLIANQFNSA | |
| | IGKIQDSLSSTASALGKLQDVVNQNAQALN | |
| | TLVKQLSSNFGAISSVLNDILSRLDKVEAE | |
| | VQIDRLITGRLQSLQTYVTQQLIRAAEIRA | |
| | SANLAAIKMSECVLGQSKRVDFCGKGYHLM | |
| | SFPQSAPHGVVFLHVTYVPAQEKNFTTAPA | |
| | ICHDGKAHFPREGVFVSNGTHWFVTQRNFY | |
| | EPQIITTDNTFVSGNCDVVIGIVNNTVYDP | |
| | LQPELDSFKEELDKYFKNHTSPDVDLGDIS | |
| | GINASVVNIQKEIDRLNEVAKNLNESLIDL | |
| | QELGKYEQYIKWPWYIWLGFIAGLIAIVMV | |
| | TIMLCCMTSCCSCLKGCCSCGSCCKFDEDD | |
| | SEPVLKGVKLHYT | |
| Spike S1 | MFVFLVLLPLVSSQCVNLRTRTQLPPAYTN | 126 |
| & S2 | SFTRGVYYPDKVFRSSVLHSTQDLFLPFFS | |
| ECD | NVTWFHAIHFSGTNGTKRFDNPVLPENDGV | |
| B.1.617.2 | YFASIEKSNIIRGWIFGTTLDSKTQSLLIV | |
| variant | NNATNVVIKVCEFQFCNDPFLDVYYHKNNK | |
| (Delta) | SWMESGVYSSANNCTFEYVSQPFLMDLEGK | |
| | QGNFKNLREFVFKNIDGYFKIYSKHTPINL | |
| | VRDLPQGFSVLEPLVDLPIGINITRFQTLL | |
| | ALHRSYLTPGDSSSGLTAGAAAYYVGYLQP | |
| | RTFLLKYNENGTITDAVDCALDPLSETKCT | |
| | LKSFTVEKGIYQTSNERVQPTESIVRFPNI | |
| | TNLCPFGEVENATRFASVYAWNRKRISNCV | |
| | ADYSVLYNSASFSTFKCYGVSPTKLNDLCF | |
| | TNVYADSFVIRGDEVRQIAPGQTGNIADYN | |
| | YKLPDDFTGCVIAWNSNNLDSKVGGNYNYR | |
| | YRLFRKSNLKPFERDISTEIYQAGSKPCNG | |
| | VEGFNCYFPLQSYGFQPTNGVGYQPYRVVV | |
| | LSFELLHAPATVCGPKKSTNLVKNKCVNFN | |
| | FNGLTGTGVLTESNKKFLPFQQFGRDIADT | |
| | TDAVRDPQTLEILDITPCSFGGVSVITPGT | |
| | NTSNQVAVLYQGVNCTEVPVAIHADQLTPT | |
| | WRVYSTGSNVFQTRAGCLIGAEHVNNSYEC | |
| | DIPIGAGICASYQTQTNSRRRARSVASQSI | |
| | IAYTMSLGAENSVAYSNNSIAIPTNFTISV | |
| | TTEILPVSMTKTSVDCTMYICGDSTECSNL | |
| | LLQYGSFCTQLNRALTGIAVEQDKNTQEVF | |
| | AQVKQIYKTPPIKDFGGFNFSQILPDPSKP | |
| | SKRSFIEDLLENKVTLADAGFIKQYGDCLG | |
| | DIAARDLICAQKFNGLTVLPPLLTDEMIAQ | |
| | YTSALLAGTITSGWTFGAGAALQIPFAMQM | |
| | AYRENGIGVTQNVLYENQKLIANQFNSAIG | |
| | KIQDSLSSTASALGKLQNVVNQNAQALNTL | |
| | VKQLSSNFGAISSVLNDILSRLDKVEAEVQ | |
| | IDRLITGRLQSLQTYVTQQLIRAAEIRASA | |
| | NLAATKMSECVLGQSKRVDFCGKGYHLMSF | |
| | PQSAPHGVVFLHVTYVPAQEKNFTTAPAIC | |
| | HDGKAHFPREGVFVSNGTHWFVTQRNFYEP | |
| | QIITTDNTFVSGNCDVVIGIVNNTVYDPLQ | |
| | PELDSFKEELDKYFKNHTSPDVDLGDISGI | |
| | NASVVNIQKEIDRLNEVAKNLNESLIDLQE | |
| | LGKYEQYIKWPWYIWLGFIAGLIAIVMVTI | |
| | MLCCMTSCCSCLKGCCSCGSCCKFDEDDSE | |
| | PVLKGVKLHYT | |
| Spike S1 | MFVFLVLLPLVSSQCVNLTTRTQLPPAYTN | 127 |
| & S2 | SFTRGVYYPDKVFRSSVLHSTQDLFLPFFS | |
| ECD | NVTWFHVISGTNGTKRFDNPVLPENDGVYF | |
| B.1.1.529 | ASIEKSNIIRGWIFGTTLDSKTQSLLIVNN | |
| variant | ATNVVIKVCEFQFCNDPFLHKNNKSWMESE | |
| (Omicron) | FRVYSSANNCTFEYVSQPFLMDLEGKQGNF | |
| | KNLREFVFKNIDGYFKIYSKHTPIIVREPE | |
| | DLPQGFSALEPLVDLPIGINITRFQTLLAL | |
| | HRSYLTPGDSSSGWTAGAAAYYVGYLQPRT | |
| | ELLKYNENGTITDAVDCALDPLSETKCTLK | |
| | SFTVEKGIYQTSNFRVQPTESIVRFPNITN | |
| | LCPFDEVENATRFASVYAWNRKRISNCVAD | |
| | YSVLYNLAPFFTFKCYGVSPTKLNDLCFTN | |
| | VYADSFVIRGDEVRQIAPGQTGNIADYNYK | |
| | LPDDFTGCVIAWNSNKLDSKVSGNYNYLYR | |
| | LERKSNLKPFERDISTEIYQAGNKPCNGVA | |
| | GENCYFPLRSYSFRPTYGVGHQPYRVVVLS | |
| | FELLHAPATVCGPKKSTNLVKNKCVNFNFN | |
| | GLKGTGVLTESNKKFLPFQQFGRDIADTTD | |
| | AVRDPQTLEILDITPCSFGGVSVITPGTNT | |
| | SNQVAVLYQGVNCTEVPVAIHADQLTPTWR | |
| | VYSTGSNVFQTRAGCLIGAEYVNNSYECDI | |
| | PIGAGICASYQTQTKSHRRARSVASQSIIA | |
| | YTMSLGAENSVAYSNNSIAIPTNFTISVTT | |
| | EILPVSMTKTSVDCTMYICGDSTECSNLLL | |
| | QYGSFCTQLKRALTGIAVEQDKNTQEVFAQ | |
| | VKQIYKTPPIKYFGGFNFSQILPDPSKPSK | |
| | RSFIEDLLENKVTLADAGFIKQYGDCLGDI | |
| | AARDLICAQKFKGLTVLPPLLTDEMIAQYT | |
| | SALLAGTITSGWTFGAGAALQIPFAMQMAY | |
| | RENGIGVTQNVLYENQKLIANQFNSAIGKI | |
| | QDSLSSTASALGKLQDVVNHNAQALNTLVK | |
| | QLSSKFGAISSVLNDIFSRLDKVEAEVQID | |
| | RLITGRLQSLQTYVTQQLIRAAEIRASANL | |
| | AATKMSECVLGQSKRVDFCGKGYHLMSFPQ | |
| | SAPHGVVFLHVTYVPAQEKNFTTAPAICHD | |
| | GKAHFPREGVFVSNGTHWFVTQRNFYEPQI | |
| | ITTDNTFVSGNCDVVIGIVNNTVYDPLQPE | |
| | LDSFKEELDKYFKNHTSPDVDLGDISGINA | |
| | SVVNIQKEIDRLNEVAKNLNESLIDLQELG | |
| | KYEQYIKWPWYIWLGFIAGLIAIVMVTIML | |
| | CCMTSCCSCLKGCCSCGSCCKFDEDDSEPV | |
| | LKGVKLHYT | |

Tables 6A and 6B show post-SELEX optimization of aptamers identified to bind Spike glycoprotein (RBD) at dU positions:

TABLE 6A

| | | SEQ | |
|---|---|---|---|
| Aptamer | | ID | |
| ID | Sequence (5′ to 3′) | NO. | K_{d} (M) |
| 26874-29_46 | GGGAFApYAYGpGYppGAppGpYpGGApG | 28 | 3.6E−10 |
| 26874-29_47 | GGGAPApYAYGpGYppGAppGpYpGGApG | 29 | 3.1E−10 |
| 26874-29_48 | GGGAYApFAYGpGYppGAppGpYpGGApG | 30 | 2.6E−10 |
| 26874-29_49 | GGGAYApPAYGpGYppGAppGpYpGGApG | 31 | 4.0E−10 |
| 26874-29_50 | GGGAYApYAFGpGYppGAppGpYpGGApG | 32 | 3.1E−10 |
| 26874-29_51 | GGGAYApYAPGpGYppGAppGpYpGGApG | 33 | 3.6E−10 |
| 26874-29_52 | GGGAYApYAYGpGFppGAppGpYpGGApG | 34 | 1.1E−10 |
| 26874-29_53 | GGGAYApYAYGpGPppGAppGpYpGGApG | 35 | 2.0E−10 |
| 26874-29_54 | GGGAYApYAYGpGYppGAppGpFpGGApG | 36 | 2.1E−10 |
| 26874-29_55 | GGGAYApYAYGpGYppGAppGpPpGGApG | 37 | 2.5E−10 |
| 26874-29_56 | GGGAWApYAYGpGYppGAppGpYpGGApG | 38 | 5.4E−10 |
| 26874-29_57 | GGGAZApYAYGpGYppGAppGpYpGGApG | 39 | 2.1E−10 |
| 26874-29_58 | GGGAYApWAYGpGYppGAppGpYpGGApG | 40 | 2.7E−10 |
| 26874-29_59 | GGGAYApZAYGpGYppGAppGpYpGGApG | 41 | 1.8E−10 |
| 26874-29_60 | GGGAYApYAWGpGYppGAppGpYpGGApG | 42 | 9.8E−11 |
| 26874-29_61 | GGGAYApYAZGpGYppGAppGpYpGGApG | 43 | 3.1E−10 |
| 26874-29_62 | GGGAYApYAYGpGWppGAppGpYpGGApG | 44 | 1.3E−10 |
| 26874-29_63 | GGGAYApYAYGpGZppGAppGpYpGGApG | 45 | 1.6E−10 |
| 26874-29_64 | GGGAYApYAYGpGYppGAppGpWpGGApG | 46 | 2.7E−10 |
| 26874-29_65 | GGGAYApYAYGpGYppGAppGpZpGGApG | 47 | 2.8E−10 |
| 26874-29_66 | GGGAF\hat{ }ApYAYGpGYppGAppGpYpGGApG | 48 | 1.9E−10 |
| 26874-29_68 | GGGAYApF\hat{ }AYGpGYppGAppGpYpGGApG | 49 | 2.1E−10 |
| 26874-29_70 | GGGAYApYAF\hat{ }GpGYppGAppGpYpGGApG | 50 | 2.0E−10 |
| 26874-29_72 | GGGAYApYAYGpGF\hat{ }ppGAppGpYpGGApG | 51 | 1.4E−10 |
| 26874-29_74 | GGGAYApYAYGpGYppGAppGpF\hat{ }pGGApG | 52 | 4.6E−10 |
| 26874-29_76 | GGGAeApYAYGpGYppGAppGpYpGGApG | 53 | 4.6E−10 |
| 26874-29_77 | GGGAEApYAYGpGYppGAppGpYpGGApG | 54 | 1.3E−10 |
| 26874-29_78 | GGGAYApeAYGpGYppGAppGpYpGGApG | 55 | 2.4E−10 |
| 26874-29_79 | GGGAYApEAYGpGYppGAppGpYpGGApG | 56 | 1.7E−10 |
| 26874-29_80 | GGGAYApYAeGpGYppGAppGpYpGGApG | 57 | 1.6E−10 |
| 26874-29_81 | GGGAYApYAEGpGYppGAppGpYpGGApG | 58 | 1.6E−10 |
| 26874-29_82 | GGGAYApYAYGpGeppGAppGpYpGGApG | 59 | 1.8E−10 |
| 26874-29 83 | GGGAYApYAYGpGEppGAppGpYpGGApG | 60 | 1.9E−10 |
| 26874-29_84 | GGGAYApYAYGpGYppGAppGpepGGApG | 61 | 4.4E−10 |
| 26874-29_85 | GGGAYApYAYGpGYppGAppGpEpGGApG | 62 | 2.9E−10 |
| 26874-29_86 | GGGAfApYAYGpGYppGAppGpYpGGApG | 63 | 2.1E−10 |
| 26874-29_87 | GGGAJApYAYGpGYppGAppGpYpGGApG | 64 | 2.5E−10 |
| 26874-29_88 | GGGAYApfAYGpGYppGAppGpYpGGApG | 65 | 1.9E−10 |
| 26874-29_89 | GGGAYApJAYGpGYppGAppGpYpGGApG | 66 | 2.0E−10 |
| 26874-29_90 | GGGAYApYAfGpGYppGAppGpYpGGApG | 67 | 1.4E−10 |
| 26874-29_91 | GGGAYApYAYGpGfppGAppGpYpGGApG | 68 | 1.7E−10 |
| 26874-29_92 | GGGAYApYAJGpGYppGAppGpYpGGApG | 69 | 1.8E−10 |
| 26874-29_93 | GGGAYApYAYGpGJppGAppGpYpGGApG | 70 | 2.4E−10 |
| 26874-29_94 | GGGAYApYAYGpGYppGAppGpfpGGApG | 71 | 4.8E−10 |
| 26874-29_95 | GGGAYApYAYGpGYppGAppGpJpGGApG | 72 | 4.2E−10 |
| 26874-29_96 | GGGAhApYAYGpGYppGAppGpYpGGApG | 73 | 2.7E−10 |
| 26874-29_97 | GGGAOApYAYGpGYppGAppGpYpGGApG | 74 | 2.9E−10 |
| 26874-29_98 | GGGAYAphAYGpGYppGAppGpYpGGApG | 75 | 2.2E−10 |
| 26874-29_99 | GGGAYApOAYGpGYppGAppGpYpGGApG | 76 | 2.4E−10 |
| 26874-29_100 | GGGAYApYAYGpGhppGAppGpYpGGApG | 77 | 4.9E−10 |
| 26874-29_101 | GGGAYApYAOGpGYppGAppGpYpGGApG | 78 | 3.6E−10 |
| 26874-29_102 | GGGAYApYAYGpGhppGAppGpYpGGApG | 79 | 4.5E−10 |
| 26874-29 103 | GGGAYApYAYGpGOppGAppGpYpGGApG | 80 | 4.9E−10 |
| 26874-29_104 | GGGAYApYAYGpGYppGAppGphpGGApG | 81 | 5.9E−10 |
| 26874-29 105 | GGGAYApYAYGpGYppGAppGpOpGGApG | 82 | 7.9E−10 |
| 26874-29_106 | GGGAnApYAYGpGYppGAppGpYpGGApG | 83 | 6.6E−10 |
| 26874-29_107 | GGGAiApYAYGpGYppGAppGpYpGGApG | 84 | 4.9E−10 |
| 26874-29_108 | GGGAYApnAYGpGYppGAppGpYpGGApG | 85 | 7.2E−10 |
| 26874-29_109 | GGGAYApiAYGpGYppGAppGpYpGGApG | 86 | 4.8E−10 |
| 26874-29 110 | GGGAYApYAnGpGYppGAppGpYpGGApG | 87 | 4.3E−10 |
| 26874-29_112 | GGGAYApYAYGpGnppGAppGpYpGGApG | 88 | 5.6E−10 |
| 26874-29_113 | GGGAYApYAYGpGippGAppGpYpGGApG | 89 | 3.2E−10 |
| 26874-29 114 | GGGAYApYAYGpGYppGAppGpnpGGApG | 90 | 8.1E−10 |
| 26874-29 115 | GGGAYApYAYGpGYppGAppGpipGGApG | 91 | 4.6E−10 |
| 26874-29_116 | GGGAsApYAYGpGYppGAppGpYpGGApG | 92 | 5.3E−10 |
| 26874-29_117 | GGGA [symbol] ApYAYGpGYppGAppGpYpGGApG | 93 | 4.2E−10 |
| 26874-29_118 | GGGAYApsAYGpGYppGAppGpYpGGApG | 94 | 3.9E−10 |
| 26874-29_119 | GGGAYAp [symbol] AYGpGYppGAppGpYpGGApG | 95 | 5.0E−07 |
| 26874-29_120 | GGGAYApYAsGpGYppGAppGpYpGGApG | 96 | 2.4E−10 |
| 26874-29_121 | GGGAYApYA [symbol] GpGYppGAppGpYpGGApG | 97 | 4.2E−10 |
| 26874-29_122 | GGGAYApYAYGpGsppGAppGpYpGGApG | 98 | 1.6E−10 |
| 26874-29_123 | GGGAYApYAYGpG [symbol] ppGAppGpYpGGApG | 99 | 5.0E−07 |
| 26874-29_124 | GGGAYApYAYGpGYppGAppGpspGGApG | 100 | 6.4E−10 |
| 26874-29_125 | GGGAYApYAYGpGYppGAppGp [symbol] pGGApG | 101 | 3.7E−10 |
| 26874-29_126 | GGGAtApYAYGpGYppGAppGpYpGGApG | 102 | 6.5E−10 |
| 26874-29_127 | GGGAc³ApYAYGpGYppGAppGpYpGGApG | 103 | 2.8E−10 |
| 26874-29_128 | GGGAYAptAYGpGYppGAppGpYpGGApG | 104 | 5.0E−07 |
| 26874-29_130 | GGGAYApYAtGpGYppGAppGpYpGGApG | 105 | 2.8E−10 |
| 26874-29_132 | GGGAYApYAYGpGtppGAppGpYpGGApG | 106 | 2.7E−10 |
| 26874-29_134 | GGGAYApYAYGpGYppGAppGptpGGApG | 107 | 1.2E−10 |
| 26874-29 136 | GGGAIApYAYGpGYppGAppGpYpGGApG | 108 | 5.7E−10 |
| 26874-29 138 | GGGAYApIAYGpGYppGAppGpYpGGApG | 109 | 5.9E−10 |
| 26874-29 140 | GGGAYApYAIGpGYppGAppGpYpGGApG | 110 | 4.7E−10 |
| 26874-29_142 | GGGAYApYAYGpGIppGAppGpYpGGApG | 111 | 3.7E−10 |
| 26874-29_144 | GGGAYApYAYGpGYppGAppGpIpGGApG | 112 | 5.7E−10 |
| 26874-29_146 | GGGAMApYAYGpGYppGAppGpYpGGApG | 113 | 6.1E−10 |
| 26874-29_147 | GGGAP³ApYAYGpGYppGAppGpYpGGApG | 114 | 4.4E−10 |
| 26874-29_148 | GGGAYApMAYGpGYppGAppGpYpGGApG | 115 | 4.6E−10 |
| 26874-29_149 | GGGAYApP³AYGpGYppGAppGpYpGGApG | 116 | 4.7E−10 |
| 26874-29_150 | GGGAYApYaMGpGYppGAppGpYpGGApG | 117 | 1.6E−08 |
| 26874-29 151 | GGGAYApYAP³GpGYppGAppGpYpGGApG | 118 | 7.8E−10 |
| 26874-29 152 | GGGAYApYAYGpGMppGAppGpYpGGApG | 119 | 3.3E−10 |
| 26874-29_153 | GGGAYApYAYGpGP³ppGAppGpYpGGApG | 120 | 1.1E−10 |
| 26874-29_154 | GGGAYApYAYGpGYppGAppGpMpGGApG | 121 | 4.3E−10 |
| 26874-29_155 | GGGAYApYAYGpGYppGAppGpP³pGGApG | 122 | 1.7E−10 |

In Table 6A, the following C-5 modified pyrimidines are shown: Y: 5(p-hydroxyphenylethyl)-1-aminocarbonyl-2′-deoxyuridine (TyrdU); p: 5-(1-Naphthylmethyl)aminocarbonyl-2′-deoxycytidine (NapdCQ; h: 5-(2-thiophenylmethyl)aminocarbonyl-2′-deoxyuridine (ThdU); O: N—(S-2-hydroxypropyl)-1-carboxamide-2′-deoxyuridine; n: 5-(N-ethylmorpholino)aminocarbonyl-2′-deoxyuridine (MOEdU); i: 5-(N-2-naphthylmethyl)aminocarbonyl-2′-deoxyuridine (2NapdU); s: 5-(N-3-benzothiophenylethyl)-1-aminocarbonyl-2′-deoxyuridine (BTdU); B^{a}: 5-[N-(4-phenoxybenzyl)carboxamide]-2′-deoxyuridine (POPdU); t: 5-(N-2-naphthylethyl)-1-aminocarbonyl-2′-deoxyuridine (2NEdU); I: 5-(N-isobutylaminocarbonyl)-2′-deoxyuridine (iBudU); M: 5-(N-3,4-methylenedioxybenzylaminocarbonyl-2′-deoxyuridine (MBndU); P³: 5-[N-(2-((1,1′-biphenyl)-4-yl)ethyl)carboxamide]-2′-deoxyuridine (BPEdU); F: 5-p-fluorobenzylaminocarbonyl-2′-deoxyuridine (FBndU); P: 5-napthylmethylaminocarbonyl-2′-deoxyuridine (NapdU); W: 5-tryptaminocarbonyl-2′-deoxyuridine (TrpdU); Z: 5-benzylaminocarbonyl-2′-deoxyuridine (BndU); F\hat{ }: 5-[N-(p-phenylbenzyl)carboxamide]-2′-deoxyuridine (PBndU); e: 5-(1-naphthylethyl)-1-aminocarbonyl-2′-deoxyuridine (NEdU); E: 5-phenylethyl-1-aminocarbonyl-2′-deoxyuridine (PEdU); f: 5-(3-benzofurylethyl)-2′-deoxyuridine (BFdU); J: 5-phenpropyl-1-aminocarbonyl-2′-deoxyuridine (PPdU).

TABLE 6B

| Aptamer ID | Curation Notes | Kd/Kd_20 | mod-dU | Description |
|---|---|---|---|---|
| 26874-29_46 | valid | 0.88 | FBndU | 5-p-fluorobenzylaminocarbonyl |
| 26874-29_47 | valid | 0.75 | NapdU | 5-napthylmethylaminocarbonyl |
| 26874-29_48 | valid | 0.63 | FBndU | 5-p-fluorobenzylaminocarbonyl |
| 26874-29_49 | valid | 0.97 | NapdU | 5-napthylmethylaminocarbonyl |
| 26874-29_50 | valid | 0.76 | FBndU | 5-p-fluorobenzylaminocarbonyl |
| 26874-29_51 | valid | 0.87 | NapdU | 5-napthylmethylaminocarbonyl |
| 26874-29_52 | valid | 0.27 | FBndU | 5-p-fluorobenzylaminocarbonyl |
| 26874-29_53 | valid | 0.49 | NapdU | 5-napthylmethylaminocarbonyl |
| 26874-29_54 | valid | 0.52 | FBndU | 5-p-fluorobenzylaminocarbonyl |
| 26874-29_55 | valid | 0.62 | NapdU | 5-napthylmethylaminocarbonyl |
| 26874-29_56 | valid | 1.3 | TrpdU | 5-tryptaminocarbonyl |
| 26874-29_57 | valid | 0.50 | BndU | 5-benzylaminocarbonyl |
| 26874-29_58 | valid | 0.67 | TrpdU | 5-tryptaminocarbonyl |
| 26874-29_59 | valid | 0.42 | BndU | 5-benzylaminocarbonyl |
| 26874-29_60 | valid | 0.24 | TrpdU | 5-tryptaminocarbonyl |
| 26874-29_61 | valid | 0.75 | BndU | 5-benzylaminocarbonyl |
| 26874-29_62 | valid | 0.30 | TrpdU | 5-tryptaminocarbonyl |
| 26874-29_63 | valid | 0.40 | BndU | 5-benzylaminocarbonyl |
| 26874-29_64 | valid | 0.65 | TrpdU | 5-tryptaminocarbonyl |
| 26874-29_65 | valid | 0.67 | BndU | 5-benzylaminocarbonyl |
| 26874-29_66 | valid | 0.47 | PBndU | 5-[N-(p-phenylbenzyl)carboxamide]-2′-deoxyuridine |
| 26874-29_68 | valid | 0.52 | PBndU | 5-[N-(p-phenylbenzyl)carboxamide]-2′-deoxyuridine |
| 26874-29_70 | valid | 0.48 | PBndU | 5-[N-(p-phenylbenzyl)carboxamide]-2′-deoxyuridine |
| 26874-29_72 | valid | 0.33 | PBndU | 5-[N-(p-phenylbenzyl)carboxamide]-2′-deoxyuridine |
| 26874-29_74 | valid low plateau | 1.1 | PBndU | 5-[N-(p-phenylbenzyl)carboxamide]-2′-deoxyuridine |
| | very faint spots | | | |
| 26874-29_76 | valid | 1.1 | NEdU | 5-(1-naphthylethyl)-1-aminocarbonyl |
| 26874-29_77 | valid | 0.32 | PEdU | 5-phenethyl-1-aminocarbonyl |
| 26874-29_78 | valid | 0.58 | NEdU | 5-(1-naphthylethyl)-1-aminocarbonyl |
| 26874-29_79 | valid | 0.42 | PEdU | 5-phenethyl-1-aminocarbonyl |
| 26874-29_80 | valid | 0.38 | NEdU | 5-(1-naphthylethyl)-1-aminocarbonyl |
| 26874-29_81 | valid | 0.39 | PEdU | 5-phenethyl-1-aminocarbonyl |
| 26874-29_82 | valid | 0.44 | NEdU | 5-(1-naphthylethyl)-1-aminocarbonyl |
| 26874-29_83 | valid | 0.47 | PEdU | 5-phenethyl-1-aminocarbonyl |
| 26874-29_84 | valid | 1.4 | NEdU | 5-(1-naphthylethyl)-1-aminocarbonyl |
| 26874-29_85 | valid low plateau | 0.71 | PEdU | 5-phenethyl-1-aminocarbonyl |
| 26874-29_86 | valid | 0.51 | BFdU | 5-(3-benzofurylethyl) |
| 26874-29_87 | valid | 0.61 | PPdU | 5-phenpropyl-1-aminocarbonyl |
| 26874-29_88 | valid | 0.45 | BFdU | 5-(3-benzofurylethyl) |
| 26874-29_89 | valid | 0.49 | PPdU | 5-phenpropyl-1-aminocarbonyl |
| 26874-29_90 | valid | 0.33 | BFdU | 5-(3-benzofurylethyl) |
| 26874-29_91 | valid | 0.41 | PPdU | 5-phenpropyl-1-aminocarbonyl |
| 26874-29_92 | valid | 0.44 | BFdU | 5-(3-benzofurylethyl) |
| 26874-29_93 | valid | 0.78 | PPdU | 5-phenpropyl-1-aminocarbonyl |
| 26874-29_94 | valid | 1.6 | BFdU | 5-(3-benzofurylethyl) |
| 26874-29_95 | valid | 1.4 | PPdU | 5-phenpropyl-1-aminocarbonyl |
| 26874-29_96 | valid | 0.66 | ThdU | 5-(2-thiophenemethyl)aminocarbonyl |
| 26874-29_97 | valid | 0.70 | ThrdU | N-(S-2-hydroxypropyl)-1-carboxamide, 2′-deoxy |
| 26874-29_98 | valid | 0.53 | ThdU | 5-(2-thiophenemethyl)aminocarbonyl |
| 26874-29_99 | valid | 0.58 | ThrdU | N-(S-2-hydroxypropyl)-1-carboxamide, 2′-deoxy |
| 26874-29_100 | valid | 1.6 | ThdU | 5-(2-thiophenemethyl)aminocarbonyl |
| 26874-29_101 | valid | 1.2 | ThrdU | N-(S-2-hydroxypropyl)-1-carboxamide, 2′-deoxy |
| 26874-29_102 | valid | 1.5 | ThdU | 5-(2-thiophenemethyl)aminocarbonyl |
| 26874-29_103 | valid | 1.6 | ThrdU | N-(S-2-hydroxypropyl)-1-carboxamide, 2′-deoxy |
| 26874-29_104 | valid | 1.9 | ThdU | 5-(2-thiophenemethyl)aminocarbonyl |
| 26874-29_105 | valid | 2.6 | ThrdU | N-(S-2-hydroxypropyl)-1-carboxamide, 2′-deoxy |
| 26874-29_106 | valid | 2.2 | MOEdU | 5-(N-ethylmorpholino)aminocarbonyl |
| 26874-29_107 | valid | 1.6 | 2NapdU | 5-(2-naphthylmethyl)aminocarbonyl |
| 26874-29_108 | valid | 2.4 | MOEdU | 5-(N-ethylmorpholino)aminocarbonyl |
| 26874-29_109 | valid | 1.6 | 2NapdU | 5-(2-naphthylmethyl)aminocarbonyl |
| 26874-29_110 | valid | 1.4 | MOEdU | 5-(N-ethylmorpholino)aminocarbonyl |
| 26874-29_111 | | | 2NapdU | 5-(2-naphthylmethyl)aminocarbonyl |
| 26874-29_112 | valid | 1.9 | MOEdU | 5-(N-ethylmorpholino)aminocarbonyl |
| 26874-29_113 | valid | 1.0 | 2NapdU | 5-(2-naphthylmethyl)aminocarbonyl |
| 26874-29_114 | valid | 2.7 | MOEdU | 5-(N-ethylmorpholino)aminocarbonyl |
| 26874-29_115 | valid | 1.5 | 2NapdU | 5-(2-naphthylmethyl)aminocarbonyl |
| 26874-29_116 | valid low plateau | 1.8 | BTdU | 5-(3-benzothiophenylethyl)-1-aminocarbonyl |
| 26874-29_117 | valid | 1.4 | POPdU | 5-[N-(4-phenoxybenzyl)carboxamide]-2′-deoxyuridine |
| 26874-29_118 | valid | 1.3 | BTdU | 5-(3-benzothiophenylethyl)-1-aminocarbonyl |
| 26874-29_119 | assigned | 1656 | POPdU | 5-[N-(4-phenoxybenzyl)carboxamide]-2′-deoxyuridine |
| 26874-29_120 | valid | 0.79 | BTdU | 5-(3-benzothiophenylethyl)-1-aminocarbonyl |
| 26874-29_121 | valid low plateau | 1.4 | POPdU | 5-[N-(4-phenoxybenzyl)carboxamide]-2′-deoxyuridine |
| 26874-29_122 | valid | 0.52 | BTdU | 5-(3-benzothiophenylethyl)-1-aminocarbonyl |
| 26874-29_123 | assigned | 1656 | POPdU | 5-[N-(4-phenoxybenzyl)carboxamide]-2′-deoxyuridine |
| 26874-29_124 | valid | 2.1 | BTdU | 5-(3-benzothiophenylethyl)-1-aminocarbonyl |
| 26874-29_125 | valid low plateau | 1.2 | POPdU | 5-[N-(4-phenoxybenzyl)carboxamide]-2′-deoxyuridine |
| 26874-29_126 | valid low plateau | 2.1 | 2NEdU | 5-(2-naphthylethyl)-1-aminocarbonyl |
| 26874-29_128 | assigned | 1656 | 2NEdU | 5-(2-naphthylethyl)-1-aminocarbonyl |
| 26874-29_130 | valid | 0.93 | 2NEdU | 5-(2-naphthylethyl)-1-aminocarbonyl |
| 26874-29_132 | valid | 0.88 | 2NEdU | 5-(2-naphthylethyl)-1-aminocarbonyl |
| 26874-29_134 | valid very low | 0.38 | 2NEdU | 5-(2-naphthylethyl)-1-aminocarbonyl |
| | plateau and faint | | | |
| | spots | | | |
| 26874-29_136 | valid | 1.9 | iBudU | 5-isobutylaminocarbonyl |
| 26874-29_138 | valid | 2.0 | iBudU | 5-isobutylaminocarbonyl |
| 26874-29_140 | valid | 1.5 | iBudU | 5-isobutylaminocarbonyl |
| 26874-29_142 | valid | 1.2 | iBudU | 5-isobutylaminocarbonyl |
| 26874-29_144 | valid | 1.9 | iBudU | 5-isobutylaminocarbonyl |
| 26874-29_146 | valid low plateau | 2.0 | MBndU | 5-(3,4-methylenedioxybenzylaminocarbonyl |
| 26874-29_147 | assigned low plateau | 1.1 | BPEdU | 5-[N-(2-((1,1′-biphenyl)-4-yl)ethyl)carboxamide]-2′-deoxyuridine |
| 26874-29_148 | valid low plateau | 1.1 | MBndU | 5-(3,4-methylenedioxybenzylaminocarbonyl |
| 26874-29_149 | valid low plateau | 1.2 | BPEdU | 5-[N-(2-((1,1′-biphenyl)-4-yl)ethyl)carboxamide]-2′-deoxyuridine |
| 26874-29_150 | valid | 38 | MBndU | 5-(3,4-methylenedioxybenzylaminocarbonyl |
| 26874-29_151 | valid | 1.9 | BPEdU | 5-[N-(2-((1,1′-biphenyl)-4-yl)ethyl)carboxamide]-2′-deoxyuridine |
| 26874-29_152 | valid | 1.1 | MBndU | 5-(3,4-methylenedioxybenzylaminocarbonyl |
| 26874-29_153 | valid | 0.28 | BPEdU | 5-[N-(2-((1,1′-biphenyl)-4-yl)ethyl)carboxamide]-2′-deoxyuridine |
| 26874-29_154 | valid | 1.4 | MBndU | 5-(3,4-methylenedioxybenzylaminocarbonyl |
| 26874-29_155 | valid | 0.40 | BPEdU | 5-[N-(2-((1,1′-biphenyl)-4-yl)ethyl)carboxamide]-2′-deoxyuridine |

Table 7 shows post-SELEX optimization of aptamers identified to bind Spike glycoprotein (RBD) at dC positions.

TABLE 7

| | | SEQ ID | |
|---|---|---|---|
| Aptamer ID | Sequence 5′ to 3′ | NO: | K_{d} M |
| 26874-29_20 | GGGAYApYAYGpGYppGAppGpYpGGApG | 7 | 4.1E−10 |
| OH-26874-29_181 | GGGAYAiYAYGpGYppGAppGpYpGGApG | 128 | 2.0E−10 |
| OH-26874-29_182 | GGGAYApYAYG [symbol] GYppGAppGpYpGGApG | 129 | 4.1E−10 |
| OH-26874-29 183 | GGGAYApYAYGpGY [symbol] pGAppGpYpGGApG | 130 | 3.8E−10 |
| OH-26874-29_184 | GGGAYApYAYGpGYp [symbol] GAppGpYpGGApG | 131 | 3.0E−10 |
| OH-26874-29 185 | GGGAYApYAYGpGYppGA [symbol] pGpYpGGApG | 132 | 4.5E−10 |
| OH-26874-29_186 | GGGAYApYAYGpGYppGAp [symbol] GpYpGGApG | 133 | 3.0E−10 |
| OH-26874-29_187 | GGGAYApYAYGpGYppGAppG [symbol] YpGGApG | 134 | 4.4E−10 |
| OH-26874-29_188 | GGGAYApYAYGpGYppGAppGpY [symbol] GGApG | 135 | 2.1E−10 |
| OH-26874-29 189 | GGGAYApYAYGpGYppGAppGpYpGGA [symbol] G | 136 | 2.5E−10 |
| OH-26874-29_190 | GGGAYArYAYGpGYppGAppGpYpGGApG | 137 | 2.0E−10 |
| OH-26874-29_191 | GGGAYApYAYGrGYppGAppGpYpGGApG | 138 | 3.6E−10 |
| OH-26874-29_192 | GGGAYApYAYGpGYrpGAppGpYpGGApG | 139 | 1.0E−06 |
| OH-26874-29_193 | GGGAYApYAYGpGYprGAppGpYpGGApG | 140 | 3.9E−10 |
| OH-26874-29 194 | GGGAYApYAYGpGYppGArpGpYpGGApG | 141 | 3.8E−10 |
| OH-26874-29_195 | GGGAYApYAYGpGYppGAprGpYpGGApG | 142 | 2.5E−10 |
| OH-26874-29_196 | GGGAYApYAYGpGYppGAppGrYpGGApG | 143 | 2.3E−10 |
| OH-26874-29_197 | GGGAYApYAYGpGYppGAppGpYrGGApG | 144 | 2.6E−10 |
| OH-26874-29_198 | GGGAYApYAYGpGYppGAppGpYpGGArG | 145 | 2.6E−10 |
| OH-26874-29_199 | GGGAYAzYAYGpGYppGAppGpYpGGApG | 146 | 3.1E−10 |
| OH-26874-29_200 | GGGAYApYAYGzGYppGAppGpYpGGApG | 147 | 3.6E−10 |
| OH-26874-29_201 | GGGAYApYAYGpGYzpGAppGpYpGGApG | 148 | 3.3E−10 |
| OH-26874-29_202 | GGGAYApYAYGpGYpzGAppGpYpGGApG | 149 | 4.1E−10 |
| OH-26874-29_203 | GGGAYApYAYGpGYppGAzpGpYpGGApG | 150 | 2.6E−10 |
| OH-26874-29_204 | GGGAYApYAYGpGYppGApzGpYpGGApG | 151 | 3.1E−10 |
| OH-26874-29_205 | GGGAYApYAYGpGYppGAppGzYpGGApG | 152 | 5.3E−10 |
| OH-26874-29_206 | GGGAYApYAYGpGYppGAppGpYzGGApG | 153 | 3.4E−10 |
| OH-26874-29_207 | GGGAYApYAYGpGYppGAppGpYpGGAzG | 154 | 5.0E−10 |
| OH-26874-29_208 | GGGAYA [symbol] YAYGpGYppGAppGpYpGGApG | 155 | 4.6E−10 |
| OH-26874-29_209 | GGGAYApYAYG [symbol] GYppGAppGpYpGGApG | 156 | 5.5E−10 |
| OH-26874-29_210 | GGGAYApYAYGpG [symbol] pGAppGpYpGGApG | 157 | 5.6E−10 |
| OH-26874-29_211 | GGGAYApYAYGpGYp [symbol] GAppGpYpGGApG | 158 | 3.4E−10 |
| OH-26874-29_212 | GGGAYApYAYGpGYppGA [symbol] pGpYpGGApG | 159 | 4.4E−10 |
| OH-26874-29_213 | GGGAYApYAYGpGYppGAp [symbol] GpYpGGApG | 160 | 3.3E−10 |
| OH-26874-29_214 | GGGAYApYAYGpGYppGAppG [symbol] YpGGApG | 161 | 3.9E−10 |
| OH-26874-29_215 | GGGAYApYAYGpGYppGAppGpY [symbol] GGApG | 162 | 3.0E−10 |
| OH-26874-29_216 | GGGAYApYAYGpGYppGAppGpYpGGA [symbol] G | 163 | 3.1E−10 |
| OH-26874-29_314 | GGGAYAf\hat{ }YAYGpGYppGAppGpYpGGApG | 164 | 3.0E−10 |
| OH-26874-29_315 | GGGAYApYAYGf\hat{ }GYppGAppGpYpGGApG | 165 | 1.6E−10 |
| OH-26874-29_316 | GGGAYApYAYGpGYf\hat{ }pGAppGpYpGGApG | 166 | 1.9E−10 |
| OH-26874-29_317 | GGGAYApYAYGpGYpf\hat{ }GAppGpYpGGApG | 167 | 3.8E−10 |
| OH-26874-29_318 | GGGAYApYAYGpGYppGAf\hat{ }pGpYpGGApG | 168 | 2.2E−10 |
| OH-26874-29_319 | GGGAYApYAYGpGYppGAppf\hat{ }GpYpGGApG | 169 | 3.2E−10 |
| OH-26874-29_320 | GGGAYApYAYGpGYppGAppGf\hat{ }YpGGApG | 170 | 2.7E−10 |
| OH-26874-29_321 | GGGAYApYAYGpGYppGAppGpYf\hat{ }GGApG | 171 | 2.1E−10 |
| OH-26874-29_322 | GGGAYApYAYGpGYppGAppGpYpGGAf\hat{ }G | 172 | 2.4E−10 |
| OH-26874-29_323 | GGGAYAp\hat{ }YAYGpGYppGAppGpYpGGApG | 173 | 2.4E−10 |
| OH-26874-29_324 | GGGAYApYAYGp\hat{ }GYppGAppGpYpGGApG | 174 | 2.7E−10 |
| OH-26874-29_325 | GGGAYApYAYGpGYp\hat{ }pGAppGpYpGGApG | 175 | 2.3E−10 |
| OH-26874-29_326 | GGGAYApYAYGpGYpp\hat{ }GAppGpYpGGApG | 176 | 2.2E−10 |
| OH-26874-29_327 | GGGAYApYAYGpGYppGAp\hat{ }pGpYpGGApG | 177 | 1.6E−10 |
| OH-26874-29_328 | GGGAYApYAYGpGYppGApp\hat{ }GpYpGGApG | 178 | 3.8E−11 |
| OH-26874-29_329 | GGGAYApYAYGpGYppGAppGp\hat{ }YpGGApG | 179 | 2.2E−10 |
| OH-26874-29 330 | GGGAYApYAYGpGYppGAppGpYp\hat{ }GGApG | 180 | 1.1E−10 |
| OH-26874-29_331 | GGGAYApYAYGpGYppGAppGpYpGGAp\hat{ }G | 181 | 1.3E−10 |
| OH-26874-29_332 | GGGAYAc\hat{ }YAYGpGYppGAppGpYpGGApG | 182 | 1.3E−10 |
| OH-26874-29_333 | GGGAYApYAYGc\hat{ }GYppGAppGpYpGGApG | 183 | 1.1E−10 |
| OH-26874-29_334 | GGGAYApYAYGpGYc\hat{ }pGAppGpYpGGApG | 184 | 2.8E−10 |
| OH-26874-29_335 | GGGAYApYAYGpGYpc\hat{ }GAppGpYpGGApG | 185 | 3.7E−10 |
| OH-26874-29_336 | GGGAYApYAYGpGYppGAc\hat{ }pGpYpGGApG | 186 | 3.8E−10 |
| OH-26874-29_337 | GGGAYApYAYGpGYppGApc\hat{ }GpYpGGApG | 187 | 2.7E−10 |
| OH-26874-29 338 | GGGAYApYAYGpGYppGAppGc\hat{ }YpGGApG | 188 | 2.2E−10 |
| OH-26874-29 339 | GGGAYApYAYGpGYppGAppGpYc\hat{ }GGApG | 189 | 1.7E−10 |
| OH-26874-29_340 | GGGAYApYAYGpGYppGAppGpYpGGAc\hat{ }G | 190 | 1.9E−10 |
| OH-26874-29_354 | GGGAUACUAUGCGUCCGACCGCUCGGACG | 191 | |

In Table 8, the following C-5 modified pyrimidines are shown: Y=Tyr-dU; r=PP-dC; z=Bn-dC; p=Nap-dC; i^{a}=2Nap-dC; Y^{a}=Tyr-dC; f\hat{ }=PBn-dC; p\hat{ }=DPP-dC; c\hat{ }=DCPE-dC.

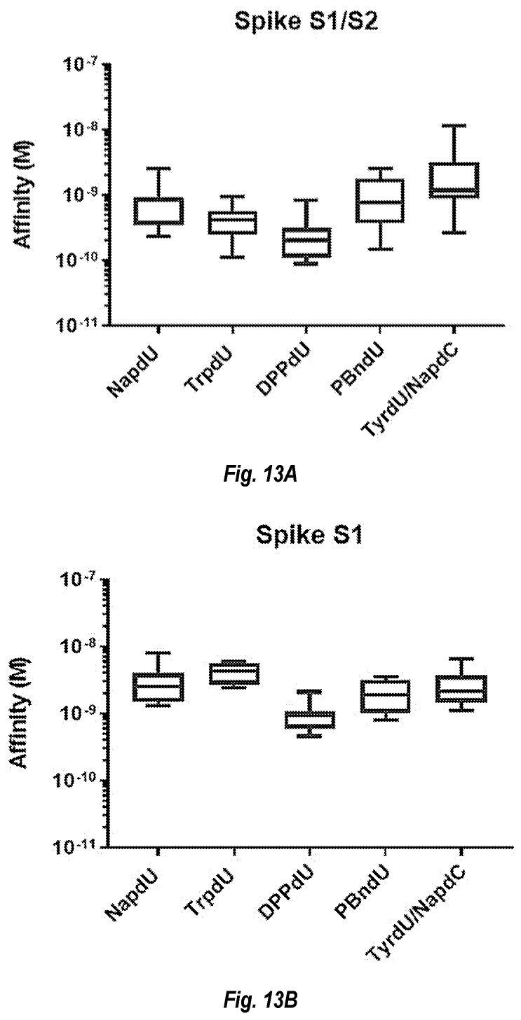

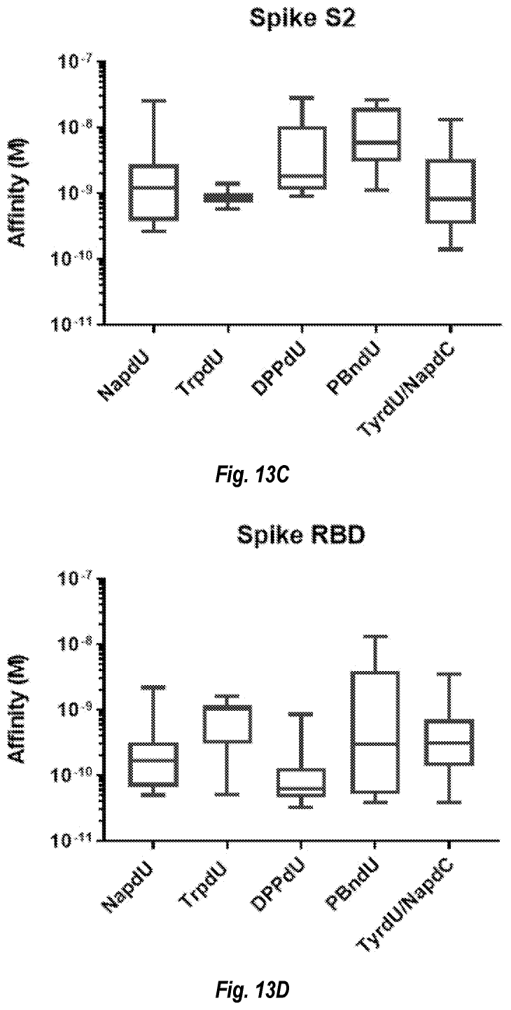

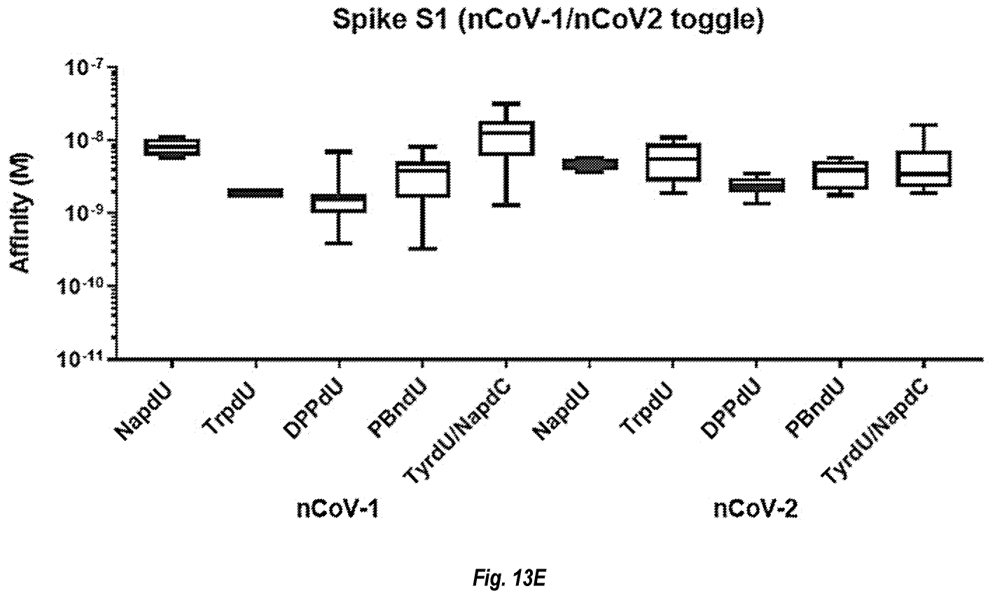

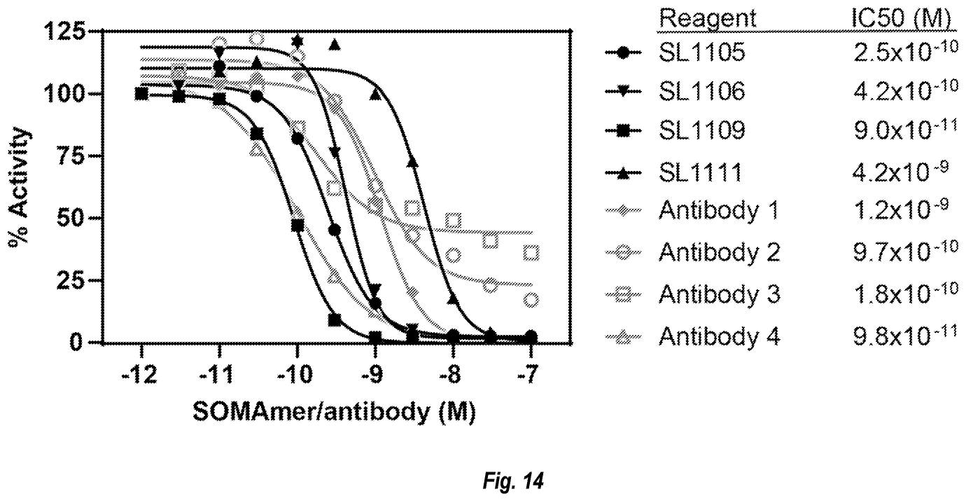

Many high affinity binders (K_{d}<10 nM) were identified from each of the 25 SELEX experiments described in Example 2 through filter binding assay evaluation (FIG. 13A-13E). Of the 287 total SOMAmer reagents screened for binding more than one third of the reagents had K_{d}<1 nM to their target protein (39 to spike S1/S2 ectodomain, 48 to spike S11 and 17 to spike S2). Moreover, the SELEX toggle strategy employed to drive binding reagents to conserved epitopes on the spike S11 domain, successfully identified 31 SOMAmer reagents with affinities less than 10 nM to both SARS-CoV and SARS-CoV-2 spike S11 (FIG. 13).

Table 8 shows post-SELEX optimization of aptamers identified to bind to spike S11 & S2 ECD

TABLE 8

| Aptamer ID | Sequence (5′ to 3′) | SEQ ID NO | K_{d} (M) |
|---|---|---|---|
| 26860-16_3 | GCATCGGAAPGAAPGPCPGCCPPGCA | 192 | 5.4E−10 |
| | CPGGPGCGGPCCGAPPGPGAGGGA | | |
| 26860-16_5 | ATCGGAAPGAAPGPCPGCCPPGCACP | 193 | 3.6E−10 |
| | GGPGCGGPCCGAPPGPGAGGGA | | |
| 26860-16_6 | CGGAAPGAAPGPCPGCCPPGCACPGG | 194 | 1.9E−9 |
| | PGCGGPCCGAPPGPGAGGGA | | |
| 26860-16_7 | GAAPGAAPGPCPGCCPPGCACPGGPG | 195 | 2.8E−9 |
| | CGGPCCGAPPGPGAGGGA | | |
| 26860-16_11 | GCATCGGAAPGAAPGPCPGCCPPGCA | 196 | 3.2E−10 |
| | CPGGPGCGGPCCGAPPGPGA | | |
| 26860-16 12 | GCATCGGAAPGAAPGPCPGCCPPGCA | 197 | 2.6E−10 |
| | CPGGPGCGGPCCGAPPGP | | |
| 26860-16 13 | GCATCGGAAPGAAPGPCPGCCPPGCA | 198 | 2.8E−10 |
| | CPGGPGCGGPCCGAPP | | |
| 26860-16_14 | GCATCGGAAPGAAPGPCPGCCPPGCA | 199 | 2.3E−10 |
| | CPGGPGCGGPCCGA | | |
| 26860-16_15 | ATCGGAAPGAAPGPCPGCCPPGCACP | 200 | 2.8E−10 |
| | GGPGCGGPCCGA | | |
| 26860-16_16 | TCGGAAPGAAPGPCPGCCPPGCACPG | 201 | 3.2E−10 |
| | GPGCGGPCCGA | | |
| 26860-16_17 | ATCGGAAPGAAPGPCPGCCPPGCACP | 202 | 2.9E−10 |
| | GGPGCGGPCC | | |
| 26860-16_18 | TCGGAAPGAAPGPCPGCCPPGCACPG | 203 | 3.0E−10 |
| | GPGCGGPCC | | |
| 26860-16 19 | ATCGGAAPGAAPGPCPGCCPPGCACP | 204 | 1.0E−9 |
| | GGPGCGGP | | |
| 26860-16_20 | TCGGAAPGAAPGPCPGCCPPGCACPG | 205 | 6.3E−10 |
| | GPGCGGP | | |
| 26860-16_28 | TCGGAATGAATGTCTGCCTTGCACTG | 206 | Not |
| | GTGCGGTCC | | determined |
| 26860-75_3 | GCATCAPCPCCCAGCPAAPGAGPGPC | 207 | 1.4E−10 |
| | PGAAPPGGACPGGPCCPCPGGGGA | | |
| 26860-75_5 | ATCAPCPCCCAGCPAAPGAGPGPCPG | 208 | 5.3E−10 |
| | AAPPGGACPGGPCCPCPGGGGA | | |
| 26860-75_6 | CAPCPCCCAGCPAAPGAGPGPCPGAA | 209 | 7.2E−10 |
| | PPGGACPGGPCCPCPGGGGA | | |
| 26860-75_7 | PCPCCCAGCPAAPGAGPGPCPGAAPP | 210 | 2.1E−9 |
| | GGACPGGPCCPCPGGGGA | | |
| 26860-75_8 | PCCCAGCPAAPGAGPGPCPGAAPPGG | 211 | 8.4E−10 |
| | ACPGGPCCPCPGGGGA | | |
| 26860-75_9 | CCAGCPAAPGAGPGPCPGAAPPGGAC | 212 | 1.0E−9 |
| | PGGPCCPCPGGGGA | | |
| 26860-75_10 | GCATCAPCPCCCAGCPAAPGAGPGPC | 213 | 5.3E−10 |
| | PGAAPPGGACPGGPCCPCPGGG | | |
| 26860-75 11 | GCATCAPCPCCCAGCPAAPGAGPGPC | 214 | 3.2E−9 |
| | PGAAPPGGACPGGPCCPCPG | | |
| 26860-75_14 | GCATCAPCPCCCAGCPAAPGAGPGPC | 215 | 2.6E−8 |
| | PGAAPPGGACPGGP | | |
| 26860-75_15 | ATCAPCPCCCAGCPAAPGAGPGPCPG | 216 | 1.9E−10 |
| | AAPPGGACPGGPCCPCPGGG | | |
| 26860-75_16 | TCAPCPCCCAGCPAAPGAGPGPCPGA | 217 | 1.6E−10 |
| | APPGGACPGGPCCPCPGGG | | |
| 26860-75_17 | ATCAPCPCCCAGCPAAPGAGPGPCPG | 218 | 1.5E−10 |
| | AAPPGGACPGGPCCPCPGG | | |
| 26860-75_18 | TCAPCPCCCAGCPAAPGAGPGPCPGA | 219 | 1.8E−10 |
| | APPGGACPGGPCCPCPGG | | |
| 26860-75_26 | TCATCTCCCAGCTAATGAGTGTCTGA | 220 | Not |
| | ATTGGACTGGTCCTCTGG | | determined |
| 26876-3_4 | CCTTGpAGGpGGpApAYGGpApYYpA | 221 | 7.7E−10 |
| | YAYpGAGpGGAAGG | | |
| 26876-3_5 | TTGpAGGpGGpApAYGGpApYYpAYA | 222 | 6.3E−10 |
| | YpGAGpGGAAGG | | |
| 26876-3_6 | GpAGGpGGpApAYGGpApYYpAYAYp | 223 | 7.0E−10 |
| | GAGpGGAAGG | | |
| 26876-3_7 | AGGpGGpApAYGGpApYYpAYAYpGA | 224 | 7.8E−10 |
| | GpGGAAGG | | |
| 26876-3_8 | GpGGpApAYGGpApYYpAYAYpGAGp | 225 | 9.8E−10 |
| | GGAAGG | | |
| 26876-3_9 | GGpApAYGGpApYYpAYAYpGAGpGG | 226 | 1.7E−8 |
| | AAGG | | |
| 26876-3_10 | CCTTGpAGGpGGpApAYGGpApYYpA | 227 | 5.6E−10 |
| | YAYpGAGpGGAA | | |
| 26876-3_11 | CCTTGPAGGpGGpApAYGGpApYYpA | 228 | 6.3E−10 |
| | YAYpGAGpGG | | |
| 26876-3_12 | CCTTGpAGGpGGpApAYGGpApYYpA | 229 | 1.0E−8 |
| | YAYpGAGp | | |
| 26876-3_13 | CCTTGpAGGpGGpApAYGGpApYYpA | 230 | 7.0E−9 |
| | YAYpGA | | |
| 26876-3_14 | CCTTGpAGGpGGpApAYGGpApYYpA | 231 | 5.7E−9 |
| | YAYp | | |
| 26876-3_15 | AGGpGGpApAYGGpApYYpAYAYpGA | 232 | 3.2E−10 |
| | GpGG | | |
| 26876-3_16 | AGGpGGpApAYGGpApYYpAYAYpGA | 233 | 5.8E−10 |
| | GpG | | |
| 26876-3_17 | GGpGGpApAYGGpApYYpAYAYpGAG | 234 | 4.1E−10 |
| | pGG | | |
| 26876-3_18 | GGpGGpApAYGGpApYYpAYAYpGAG | 235 | 4.9E−10 |
| | pG | | |
| 26876-3_20 | GGpGGpApAYGGpApYYpAYAYpGAG | 236 | 5.2E−10 |
| | p | | |
| 26876-3_21 | GpGGpApAYGGpApYYpAYAYpGAGp | 237 | 3.0E−10 |
| 26876-3_36 | GGCGGCACATGGCACTTCATATCGAG | 238 | Not |
| | CG | | determined |
| 26876-13_4 | CCTTGYGpAApGpAppYYAYYpGGpYY | 239 | 1.2E−9 |
| | GAAYGYGAGAAGG | | |
| 26876-13_5 | TTGYGpAApGpAppYYAYYpGGpYYG | 240 | 9.0E−10 |
| | AAYGYGAGAAGG | | |
| 26876-13_6 | GYGpAApGpAppYYAYYpGGpYYGAA | 241 | 1.8E−9 |
| | YGYGAGAAGG | | |
| 26876-13_10 | CCTTGYGpAApGpAppYYAYYpGGpYY | 242 | 1.8E−9 |
| | GAAYGYGAGAA | | |
| 26876-13_11 | CCTTGYGpAApGpAppYYAYYpGGpYY | 243 | 4.0E−9 |
| | GAAYGYGAG | | |
| 26876-13_12 | CCTTGYGpAApGpAppYYAYYpGGpYY | 244 | 1.8E−9 |
| | GAAYGYG | | |
| 26876-13_13 | CCTTGYGpAApGpAppYYAYYpGGpYY | 245 | 6.3E−9 |
| | GAAYG | | |
| 26876-13_14 | CCTTGYGpAApGpAppYYAYYpGGpYY | 246 | 4.5E−9 |
| | GAA | | |
| 26876-13_15 | GYGpAApGpAppYYAYYpGGpYYGAA | 247 | 2.6E−10 |
| | YGYG | | |
| 26876-13_16 | YGpAApGpAppYYAYYpGGpYYGAAY | 248 | 5.0E−10 |
| | GYG | | |
| 26876-13_17 | GYGpAApGpAppYYAYYpGGpYYGAA | 249 | 4.9E−10 |
| | YGY | | |
| 26876-13_18 | YGpAApGpAppYYAYYpGGpYYGAAY | 250 | 3.7E−10 |
| | GY | | |
| 26876-13_26 | TGCAACGCACCTTATTCGGCTTGAAT | 251 | Not |
| | GT | | determined |

In Table 8, the following C-5 modified pyrimidines are shown: Y: 5-(p-(hydroxyphenethyl)-1-aminocarbonyl-2′-deoxyuridine; p: 5-(1-Naphthylmethyl)aminocarbonyl-2′-deoxycytidine

Table 9 shows aptamer binding to mutated spike protein and variants of concern

TABLE 9

| Aptamer ID | Protein | K_{d} (M) |
|---|---|---|
| 26860-16_3 | Spike RBD | 6.1E−11 |
| 26860-16_3 | Spike S1 | 4.8E−10 |
| 26860-16_3 | Spike S1 & S2 ECD | 7.1E−10 |
| 26860-16_3 | Spike S1 D614G | 2.5E−10 |
| 26860-16_18 | Spike S1 & S2 ECD | 7.1E−10 |
| 26860-16_18 | Spike S1 &S2 ECD trimer | 1.0E−9 |
| 26860-16_18 | Spike S1 & S2 ECD trimer P.1 variant | 5.8E−10 |
| 26860-16_18 | Spike S1 &S2 ECD trimer B.1.351 variant | 6.1E−10 |
| 26860-16_18 | Spike S1 &S2 ECD trimer B.1.1.7 variant | 1.0E−9 |
| 26860-16_18 | Spike S1 &S2 ECD trimer B.1.617.2 variant | 7.6E−10 |
| 26860-16_18 | Spike S1 &S2 ECD trimer B.1.1.529 variant | 5.2E−10 |
| 26860-75_3 | Spike RBD | 2.3E−10 |
| 26860-75_3 | Spike S1 | 2.8E−9 |
| 26860-75_3 | Spike S1 & S2 ECD | 5.4E−10 |
| 26860-75_3 | Spike S1 &S2 ECD trimer | 2.2E−10 |
| 26860-75_18 | Spike S1 &S2 ECD | 5.3E−10 |
| 26860-75_18 | Spike S1 &S2 ECD trimer | 8.8E−10 |
| 26860-75_18 | Spike S1 & S2 ECD trimer P.1 variant | 4.8E−10 |
| 26860-75_18 | Spike S1 &S2 ECD trimer B.1.351 variant | 6.4E−10 |
| 26860-75_18 | Spike S1 &S2 ECD trimer B.1.1.7 variant | 1.4E−9 |
| 26860-75_18 | Spike S1 &S2 ECD trimer B.1.617.2 variant | 7.4E−10 |
| 26860-75_18 | Spike S1 &S2 ECD trimer B.1.1.529 variant | 7.9E−10 |
| 26876-3_4 | Spike S2 | 3.7E−10 |
| 26876-3_4 | Spike S1 &S2 ECD | 9.2E−10 |
| 26876-3_18 | Spike S1 &S2 ECD | 9.1E−10 |
| 26876-3_18 | Spike S1 &S2 ECD trimer | 1.9E−9 |
| 26876-3_18 | Spike S1 & S2 ECD trimer P.1 variant | 1.2E−9 |
| 26876-3_18 | Spike S1 &S2 ECD trimer B.1.351 variant | 5.9E−10 |
| 26876-3_18 | Spike S1 &S2 ECD trimer B.1.1.7 variant | 1.9E−9 |
| 26876-3_18 | Spike S1 &S2 ECD trimer B.1.617.2 variant | 3.3E−9 |
| 26876-3_18 | Spike S1 &S2 ECD trimer B.1.1.529 variant | 8.0E−10 |
| 26876-13_4 | Spike S2 | 1.9E−10 |
| 26876-13_18 | Spike S1 &S2 ECD | 3.6E−10 |
| 26876-13_18 | Spike S1 &S2 ECD B.1.1.7 variant | 3.7E−10 |
| 26874-29_20 | Spike S1 & S2 ECD trimer P.1 variant | 3.9E−10 |
| 26874-29_20 | Spike S1 &S2 ECD trimer B.1.351 variant | 2.2E−10 |
| 26874-29_20 | Spike S1 &S2 ECD trimer B.1.1.7 variant | 9.1E−10 |
| 26874-29_20 | Spike S1 &S2 ECD trimer B.1.617.2 variant | 6.0E−10 |
| 26874-29_20 | Spike S1 &S2 ECD trimer B.1.1.529 variant | 4.9E−11 |

### Example 4: ACE2:SARS-CoV-2 Spike Inhibition Assay

This example provides the method used herein to measure aptamer-coronavirus protein half maximal inhibitory concentrations and to determine IC50 in a plate-based inhibition assay. The spike RBD modified aptamer 26874-29_4 was tested in an ACE2:SARS-CoV-2 Spike inhibition assay (BPS Bioscience, cat #79936) to determine if the aptamer could interfere with SARS-CoV-2 spike protein binding its receptor, ACE2. Such ability could prevent the SARS-CoV-2 virus from entering human cells. The inhibition assay was performed following the manufacturer's protocol with a few exceptions. ACE2-His protein was diluted to 1 ug/ml and 50 uL was added to 12 wells of the provided Ni-coated 96-well microplate. Modified aptamer was tested at protein concentrations ranging from 10⁻⁷ to 10⁻¹² M and added to the ACE2 containing wells prior to the addition of 0.25 nM spike RBD mFC tag protein. Secondary HRP-labeled antibody was diluted 1:1000 in the provided blocking buffer and added to every well. The provided ELISA ECL substrates were mixed 1:1 and added to every well just prior to reading the plate in chemiluminescence mode on a Spectramax (Molecular Devices). The buffer used in the assay was 1XSB18T with the addition of 2.5 mM MgCl2. All incubations were done at room temperature for 1 hour with slow shaking. After each incubation the plate was washed 3× with the provided 1× Immuno Buffer and blocked for 10 minutes with the provided Blocking Buffer and then washed 3× again. All volumes of reagents corresponded to those recommended by the manufacturer. Data were analyzed as follows: the reading from the blank well (ACE2 only well) was subtracted from all data points. The percent activity for the modified aptamer was determined by calculating the fraction of the positive control signal (ACE2 plus spike RBD). Data were plotted in GraphPad Prism and fit to a four parameter dose response curve.

Using the inhibition assay described above it was determined that Aptamer ID 26874-29_4 is capable of inhibiting spike RBD protein from binding the ACE2 receptor in a dose-dependent manner, with an EC50 of 4.2×10⁻⁹ M. The results of the inhibition assay are displayed in Table 10.

TABLE 10

| | | SEQ | | |
|---|---|---|---|---|
| Aptamer | Sequence | ID | | EC50 |
| ID | (5′ to 3′) | NO | Protein | (M) |
| 26874- | CCTTGGGAYApYAYGpGYpp | 6 | Spike | 4.2E−9 |
| 29_4 | GAppGpYpGGApGGAGAAGG | | RBD | |

87 additional SOMAmer reagents with affinity to RBD below 10 nM were tested in an ACE2:SARS-CoV-2 Spike inhibition assay (BPS Bioscience, cat #79936) to determine if the aptamer could interfere with SARS-CoV-2 spike protein binding its receptor, ACE2. Given the high affinity of the SOMAmer reagents to the spike RBD protein, low concentrations of the RBD-Fc fusion protein (250 μM) were used, allowing theoretical measurement of IC50 values as low as 125 μM. The inhibition assay was performed following the manufacturer's protocol with the few exceptions described above for the spike RBD modified aptamer 26874-29_4.

Among the 87 SOMAmer reagents tested, 36 reagents displayed a concentration-dependent inhibition of receptor binding with IC50=0.09-20 nM. For comparison, four commercial anti-Spike S1 monoclonal antibodies were included and many SOMAmer reagents inhibited the spike/ACE2 interaction with equal or greater potency than the antibodies. Representative inhibition curves are illustrated in FIG. 13A-13E.

### Example 5: Foci Reduction Neutralization Assay

This example provides the method used herein to measure aptamer-SARS-CoV-2 virus half-maximal inhibitory concentrations and to determine IC50 in a foci reduction neutralization assay. Day 1: Known virus concentration (equivalent to 100 foci forming units) is incubated with a SOMAmer test reagent for 30 min at room temperature (1:1 volume). Vero cells at a known concentration are seeded onto each well and incubated for 2 h at 37° C. (each compound is tested in triplicate). Overlay (1.5% carboxymethyl cellulose) is added and plates are incubated for additional 18 h at 37° C. Day 2: Media is carefully removed and virus is washed once with PBS. Cells are fixed with 4% paraformaldehyde and plaque forming units are counted and compared to controls.

To assess the efficacy of aptamers 26874-29_4 and 26874-29_20 at inhibiting authentic SARS-CoV-2 virus from infecting human cells these aptamers in a foci reduction neutralization assay. A 12-point titration of aptamer reagents 26874-29_4 and 26874-29_20 over a concentration range of 10⁻⁶-10⁻¹⁰ M were evaluated.

| | | SEQ | |
|---|---|---|---|
| Aptamer | | ID | IC50 |
| ID | Sequence (5′ to 3′) | NO: | (M) |
| 26874- | CCTTGGGAYApYAYGpGYpp | 6 | 1.2E−9 |
| 29_4 | GAppGpYpGGApGGAGAAGG | | |
| 26874- | GGGAYApYAYGpGYppGApp | 7 | 1.1E−9 |
| 29_20 | GpYpGGApG | | |

Additional SOMAmer reagents exhibit potent inhibitory activity against the authentic virus in vitro.

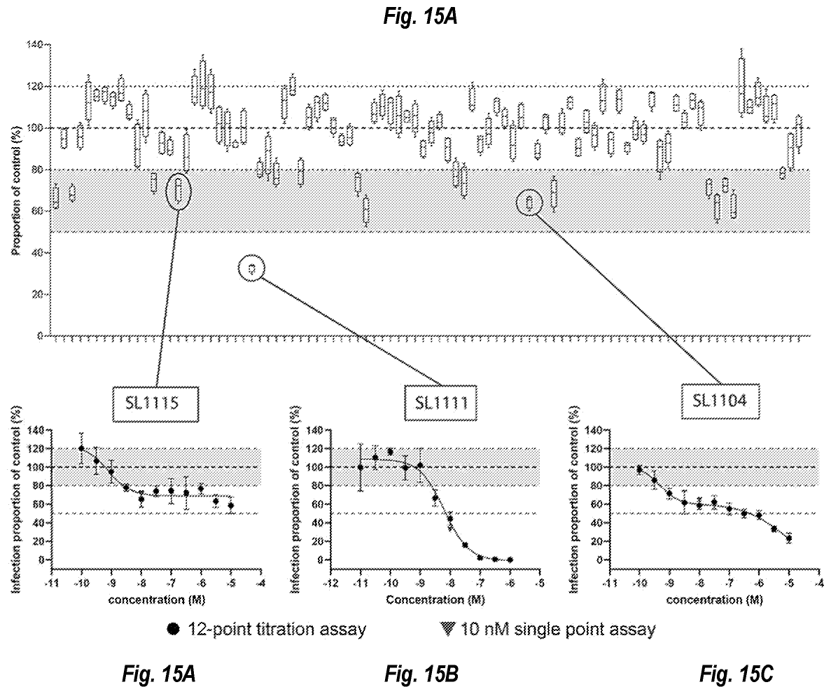

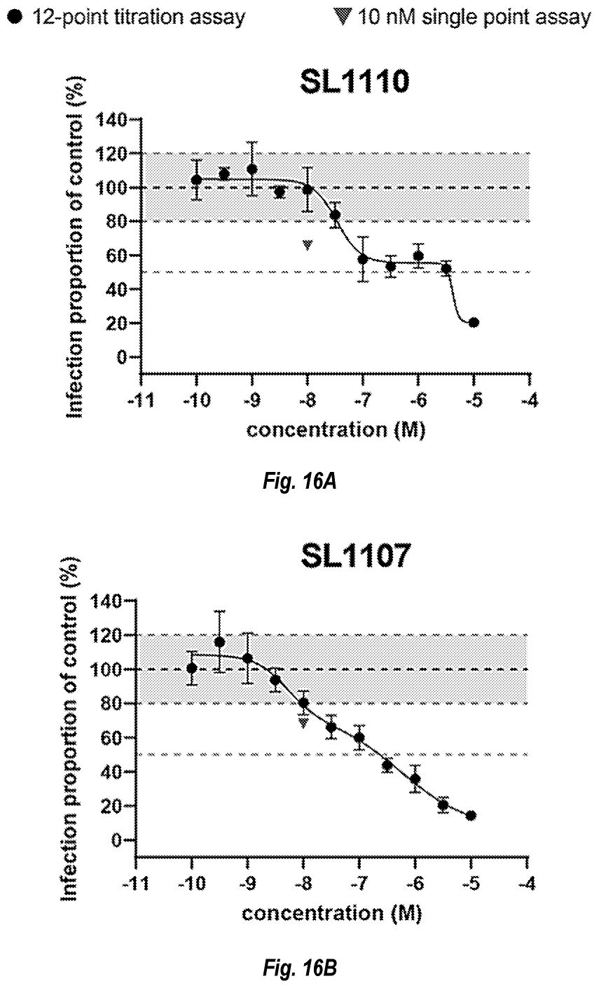

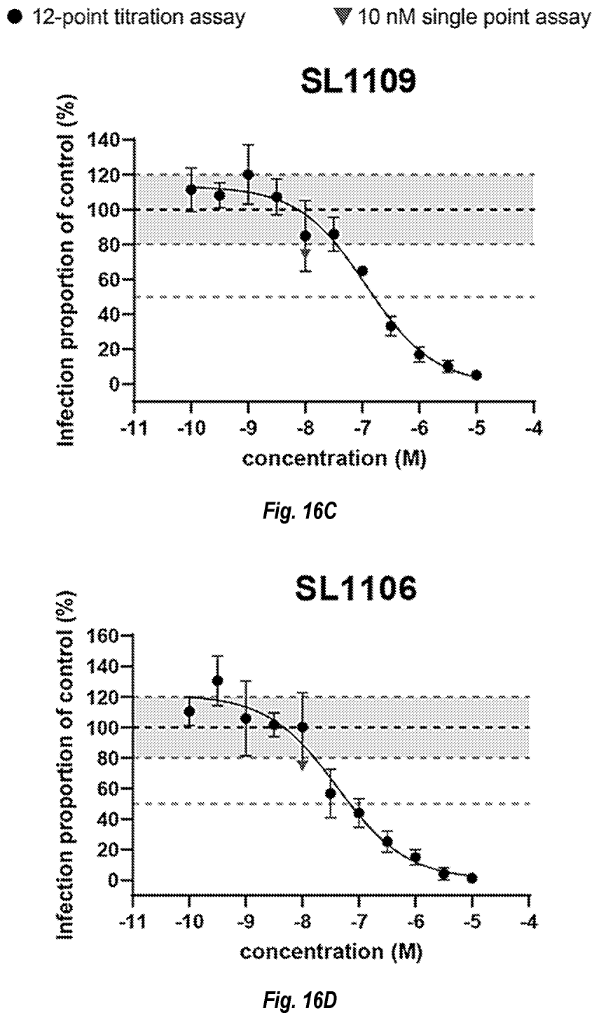

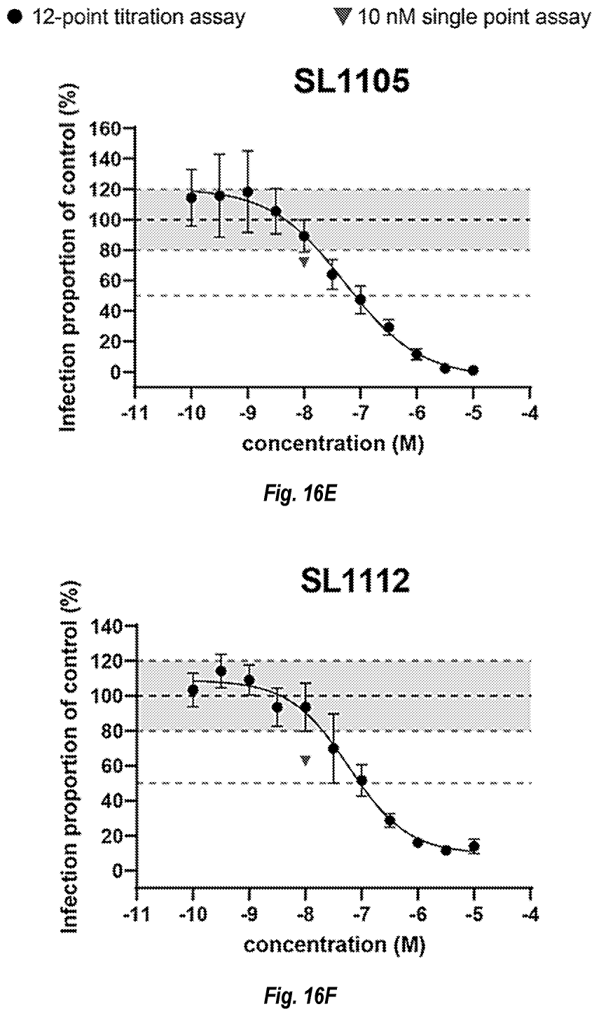

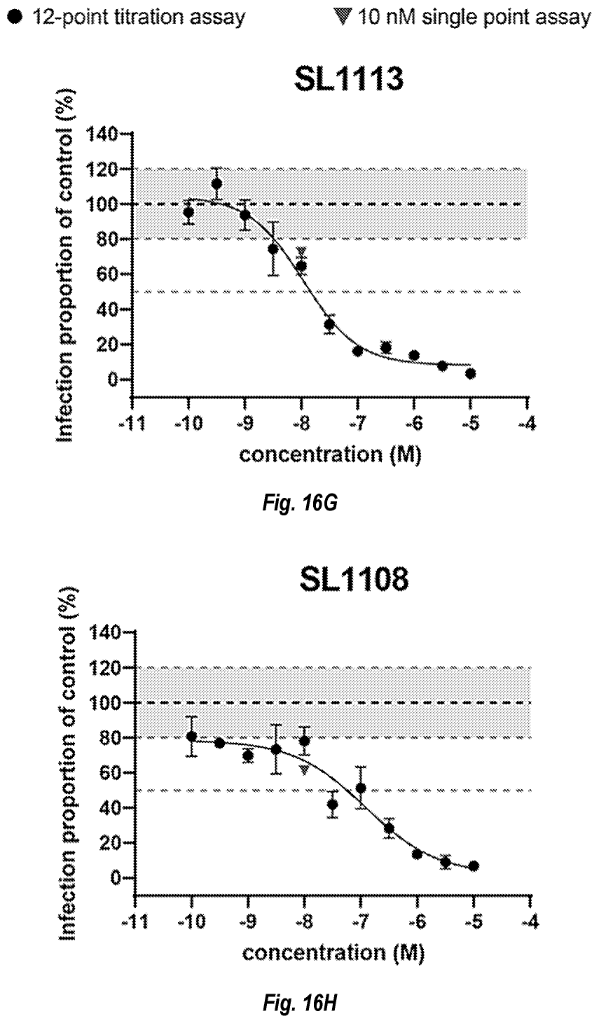

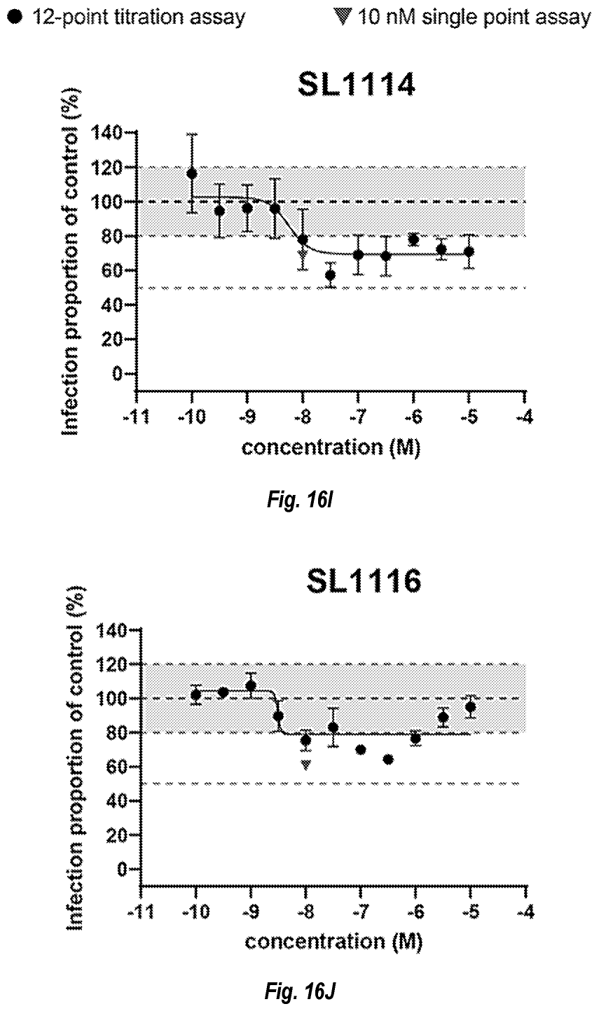

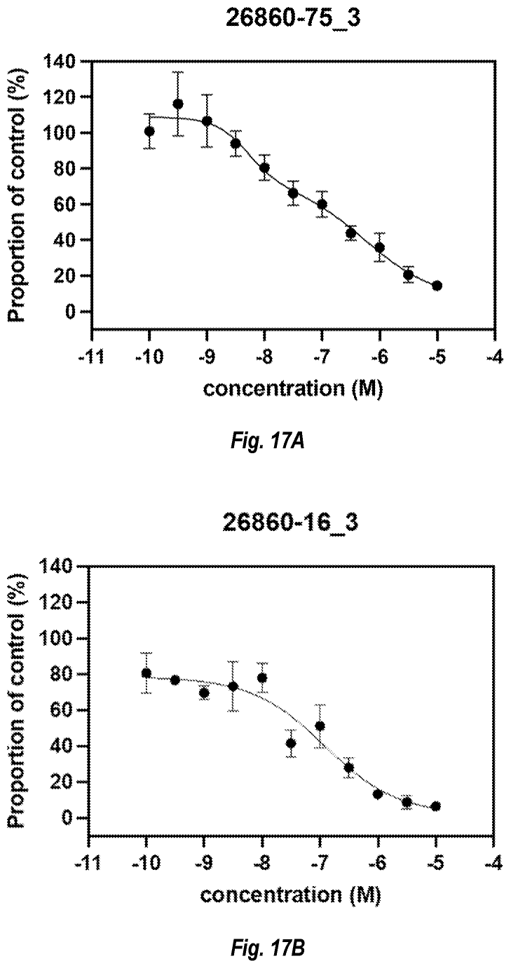

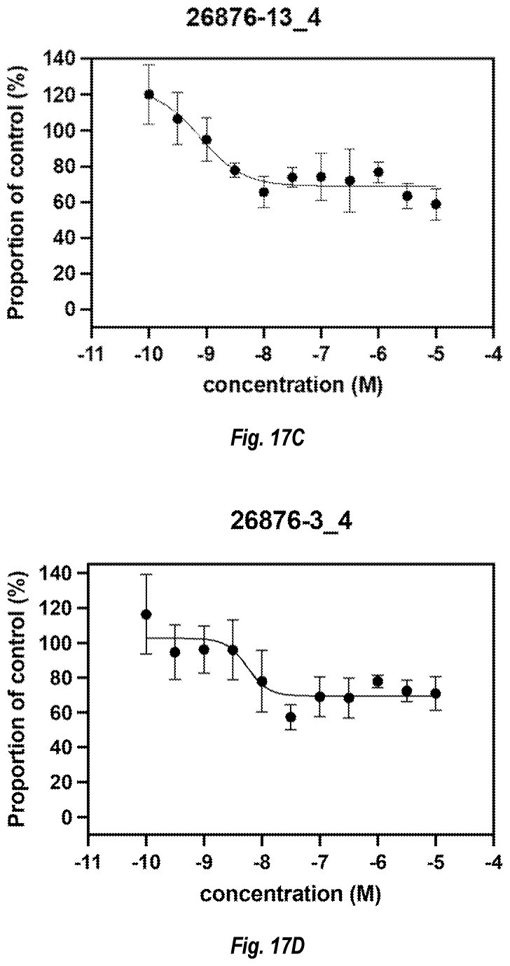

To assess the ability of a representative set of SOMAmer reagents to inhibit authentic SARS-CoV-2 virus from infecting epithelial cells expressing the ACE2 receptor (Vero cells), 92 SOMAmers were evaluated in a focus-reduction neutralization assay against an isolate representing the first pandemic wave of SARS-CoV-2 (VIC01, Pango Clade B). For this screen, the 36 reagents that effectively blocked the spike/ACE2 interaction in the sandwich assay were included, as well as 56 additional reagents that exhibited high affinity binding to spike S2 and spike S1 outside the RBD or within the RBD but lacking direct ACE2 inhibition. These reagents were chosen to cover a broad range of epitopes on the SARS-CoV-2 spike protein to explore all mechanisms of potential inhibition of viral entry including through indirect occlusion of receptor binding or through interference with the spike protein conformational changes necessary for infection. In addition, SOMAmer reagents were included with each type of chemical modification to increase the likelihood of occupying a broad range of epitopes on the spike protein. Initial screens were conducted at a single concentration of 10 nM for each SOMAmer reagent and were performed in triplicate with a fixed concentration of virus (100 foci). The number of plaque-forming units in test wells was counted and the effective neutralization threshold was set at a 20% reduction in plaque count compared to controls (FIG. 15A). The single point tests resulted in 13 SOMAmer reagents clearly in the effective neutralization range with one reagent, SL1111, distinctly exhibiting the most compelling inhibitory potency (70% reduction in infectivity). The single point assay was followed by a 12-point titration of the 13 SOMAmer reagents over a concentration range of 10⁻⁶-10⁻¹⁰ M (SL1111) and 10⁻⁵-10⁻¹¹ M for the remaining 12 reagents. Most of the SOMAmer reagents that bind the spike RBD were effective at reducing infectivity over the titration range and produced inhibition curves with Hill slopes of −1 (FIG. 15B and FIG. 16A-16J). A subset of SOMAmer reagents had clearly biphasic inhibition curves, indicating there is a population of virus with conformationally-available spike epitopes which the SOMAmer reagents can access for neutralization, and a subset of less accessible epitopes, perhaps due to the diversity of receptor conformations on the surface. Reagents targeting spike S2 showed very little neutralization of infectivity. SL1111, which was selected to bind the spike RBD, was confirmed as the most potent inhibitor of viral infection in this assay, with IC₅₀=1.2 nM (FIG. 15B). FIG. 17A-17D show the results of a foci-reduction neutralization assay for 26860-75_3 (17A), 26860-16_3 (17B), 26876-13_4 (17C), 26876-3_4 (17D).

### Example 6: Heterodimer Constructs

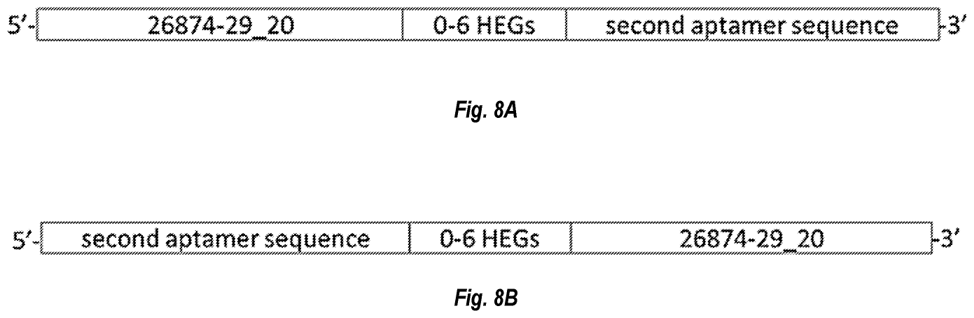

The 26874-29_20 aptamer is a potent inhibitor of SARS-CoV-2 virus, as demonstrated in the foci neutralization assay. This aptamer binds the receptor binding domain of SARS-CoV-2 spike protein, a region of the protein mutated in more virulent and infective strains of the virus currently in circulation e.g. B.1.1.7 UK variant. Random viral mutations can have other advantageous outcomes for the virus, such as providing a mechanism for therapeutic resistance. One way to reduce the chance of mutations leading to drug resistance is to have two distinct aptamers that concurrently occupy unique binding sites on the spike protein. Such drug resistance would require simultaneous mutations of the protein at both sites, the probability of this event being the product of individual probabilities. To this end, we disclose a reagent consisting of aptamer 26874-29_20 covalently attached through a flexible linker, such as consecutive hexaethylene glycols (HEG), to a second aptamer that binds a non-overlapping site on spike. The number of HEGs required in the linker are determined empirically through binding assays in which a dramatic increase in potency is observed when the two binding sites are simultaneously occupied. This avidity effect results when the optimal linker length is determined. The affinity of the heterodimer aptamer is equal to the product of the individual affinities. In certain aspects, the 26874-29_20 aptamer would be on the 5′ end of the heterodimer (FIG. 8A). In certain aspects, the 26874-29_20 aptamer would be on the 3′ end of the heterodimer (FIG. 8B). Neutralizing activity of the heterodimers in the foci neutralization assay is assessed to determine the optimal candidate.

### Example 7: Competition Binding

Modified aptamer SL1111 was 5′ end-labeled using T4 polynucleotide kinase (New England Biolabs) and 7-[32P]ATP (Perkin-Elmer). Competition assays were performed by incubating radiolabeled SL1111 (20 nM) and cold competitor SOMAmer reagents, at concentrations ranging from 10⁻⁵ to 10⁻¹⁰ M with recombinant monomeric spike S1/S2 protein (Sino Biological) (10 nM) in SB18T buffer at 37° C. for 60 minutes. Following incubation, an equal volume of 10 mM dextran sulfate was added and bound complexes were partitioned on His-tag Dyna beads at 37° C. shaking at 1850 RPMs for 5 minutes and then captured on Durapore filter plates (EMD Millipore). The fraction of SL1111 bound was quantified with a PhosphorImager (Typhoon, GE Healthcare) and data were analyzed in ImageQuant (GE Healthcare). Equilibrium dissociation constants (Kᵢ) for the competitors were determined by nonlinear regression analysis using the equation:

$$log {EC}_{50} = log [ {10}^{logKi} * ( 1 + \frac{Radio ligand ( nM )}{Hot Kd ( nM )} ) ]$$

### (GraphPad Prism).

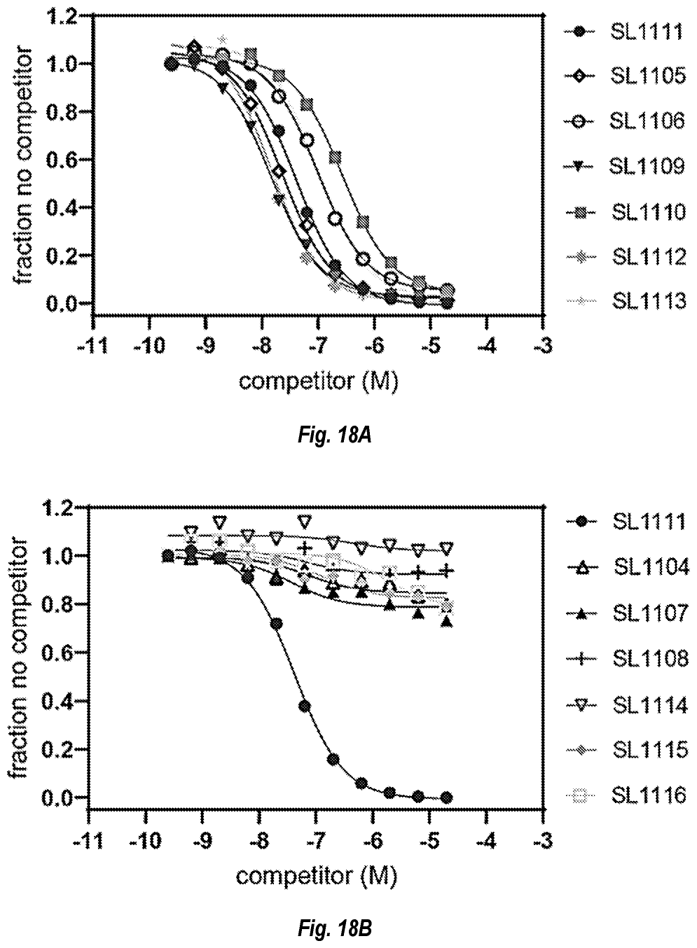

Competition assays were performed with SL1111 and the other 12 reagents to identify the SOMAmers that bind different epitopes on the spike protein and do not compete with SL1111 for binding spike. We found that six of the 12 reagents directly competed with SL1111 for binding spike S1/S2 (FIG. 18A). All six of these SOMAmers also bind the spike RBD. The remaining six SOMAmers did not compete with SL1111 for binding spike S1/S2 protein (FIG. 18B) and of these SL1107, SL1108, SL1114, and SL1115 were chosen to pursue for further optimization. These four reagents were selected based on their affinities, modifications and epitopes on spike protein they target; SL1107 and SL1108 target spike RBD outside the SL1111 binding epitope while SL1114 and SL1115 target spike S2.

As a first means of optimization, truncation studies were performed with the five lead reagents to identify the shortest sequences required for high affinity binding to spike S1/S2. For each reagent, a series of sequences were synthesized, sequentially removing nucleotides from the 5′ and 3′ ends, and the binding affinities for each were measured in a filter binding assay. The three dual modification containing sequences were highly amenable to truncation and resulted in final high affinity binding reagents that were 28 nucleotides (SL1114_18 and SL1115_18) and 29 nucleotides in length (SL1111_20). The single modification sequences were more resistant to truncation but could be reduced to a 35-mer (SL1108_18) and 44-mer (SL1107_18). See Table 8 for binding affinities.

Truncated SOMAmer reagents bind to mutated spike protein and inhibit variants of concern in vitro

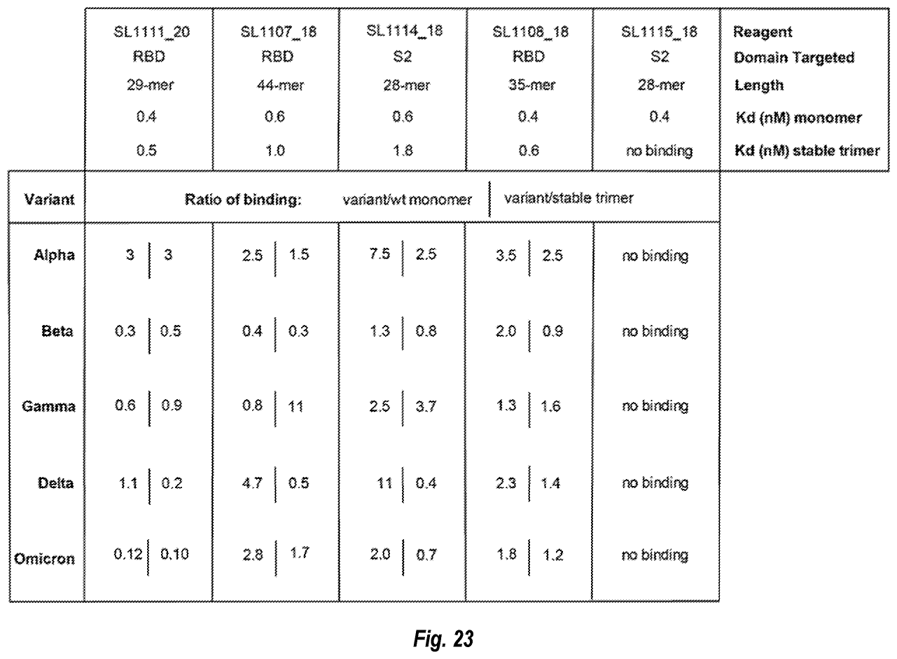

Given the pervasiveness of the Delta variant and the origins and the high transmissibility of the newly emergent Omicron variant, the five truncated lead reagents were investigated to determine whether these reagents maintain high affinity binding to recombinant spike protein monomer and stabilized trimer containing the mutations found in the five VOCs. Remarkably, no significant loss in binding affinity was observed with four of our five SOMAmer reagents for binding all five spike trimer variants of concern, compared to unmutated spike monomer and trimer proteins. These reagents include SL1111_20, the other two RBD binding reagents, SL1107_18 and SL1108_18, as well as the S2 targeting reagent SL1114_18. Conversely, the SL1115_18 reagent had no measurable binding affinity for recombinant spike trimer, up to 50 nM protein concentration, and therefore does not bind any of the recombinant spike trimer variants. The observed lack of binding affinity could be due to occlusion of the binding epitope upon spike trimerization or the presence of many stabilizing mutations inserted into the recombinant trimer protein. These include a T4 fibritin trimerization motif as well as several mutations in the S2 domain (F817P, A892P, A899P, A942P, K986P, V987P, R683A and R685A) and the deletion of the furin cleavage site at the S1/S2 boundary 21. All the recombinant spike trimer proteins used in our binding studies include these stabilizing mutations. See FIG. 23.

### Example 7: Competition Elisa

The competition ELISA used to determine the binding epitope of the aptamer was done as previously described with slight modification (Rijal et al., 2019 Cell Reports, Huang et al., 2021 Plos Pathogen). Briefly, 50 ng of affinity purified HexaPro Spike (Wuhan) protein, diluted in 1×PBS) were coated on 96-well Thermo MaxiSorp plates for 2 hours at RT, washed with 1×PBS and blocked with 300 μL of 5% (w/v) dried skim milk in 2×PBS overnight at 4° C. The competing mAbs (diluted to 5 μg/mL in 1×PBS/0.1% BSA) in quadruplicates were mixed with SL1111_20 (in 10-fold molar excess) in a round-bottomed 96-well plate and transferred to the blocked and washed plates coated with HexaPro Spike. Three wells containing the respective antibody only (no aptamer) were included to obtain the maximum binding of each of the monoclonal antibody. Wells containing aptamer was included to obtain the minimum binding (assay background). The wwPBD accession codes for the mAbs used in the assays are; EY-6A (6ZER), CR3022 (6W41), VHH72 (6WAQ), REGN10987 (6XDG), S309 (6WPS), FD-11A (7PZQ), FD-5D (7PR0), C121 (7K8Y), REGN10933 (6XDG), S2X-259 (7M72) and FI-3A (7Q0G). The plates were incubated for 1 hour at RT. Plates were then washed and 50 μL of secondary antibodies (Rabbit Anti-Human, HRP-conjugated, Dako P021402-2) diluted 1:1,600 in 1×PBS/0.1% BSA was added and the plates were incubated for another hour at RT. Plates were then washed with 1×PBS and developed by adding POD substrate (11484281001, Roche) for 5 minutes before stopping the reaction with 1M H₂SO₄. Absorbance OD₄₅₀ was measured using a Clariostar plate reader (BMG Labtech). Competition was measured using the formula below.

$$\% of blocking = \frac{X - mean minimum binding}{mean maximum binding - mean minimum binding}$$

$$X = OD 450 of the competing mAb in the presence of aptamers$$

SL1111_20 Binds to a Conserved Site of RBD Distinct from Antibody Epitopes and Potently Neutralizes Omicron

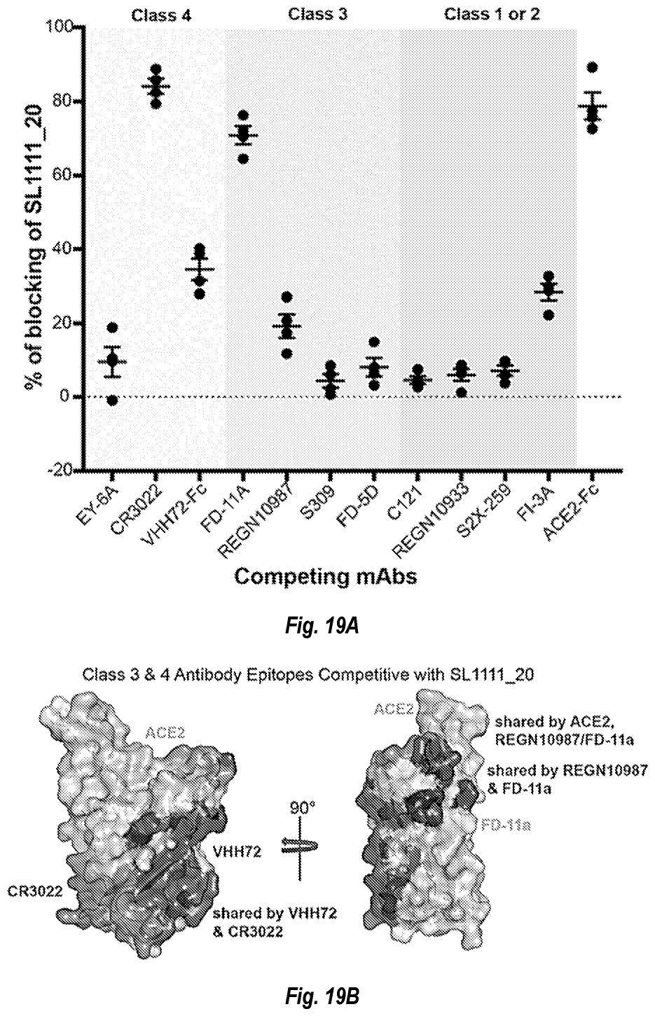

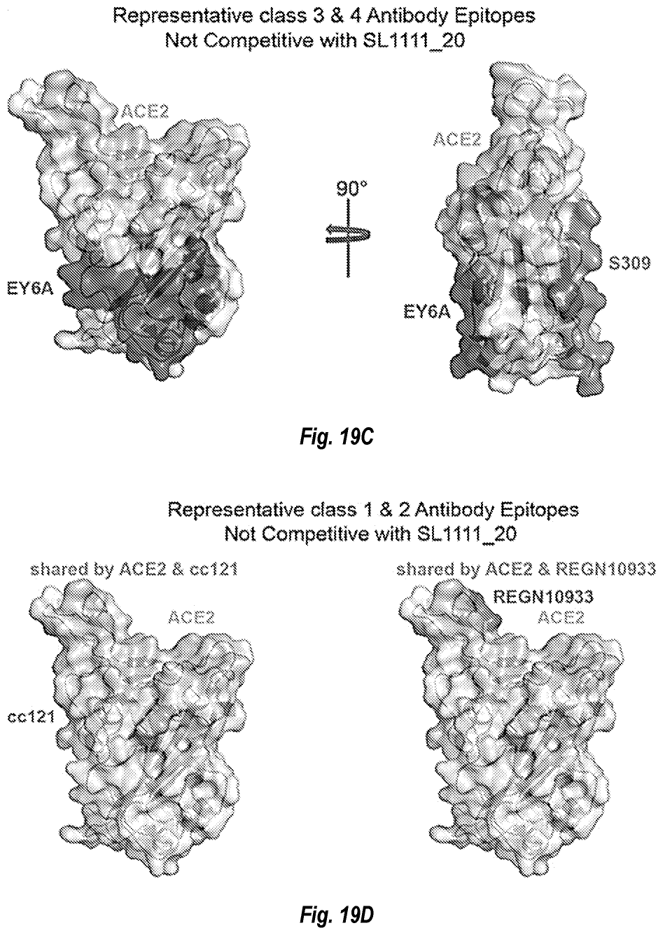

The substitutions found in the spike protein of Omicron result in a substantial escape from neutralization by antibodies generated by immune responses to earlier variants of SARS-CoV-2 (Cao et al. Nature doi.org/10.1038/s41586-021-04385-3 (2021)). The ability of the lead neutralizing aptamer to bind to the RBD of Omicron was very encouraging compared to the antibodies. In order to understand the basis for this cross-reactivity, SL1111_20 was tested to determine whether its binding site corresponded to that of a panel of structurally well-characterized antibodies, using a competition ELISA. The results show that SL1111_20 modestly competes for RBD binding with one Class 1 antibody, FI-3A, and none of the class 2 antibodies tested. (FIG. 19A) Both these classes represent the most potent, ACE2-competitive neutralizing antibodies. Robust inhibition of binding to spike RBD in the presence of SL1111_20 was observed for the Class 3 antibody, FD-11A and the Class 4 antibody, CR3022, with a less substantial effect observed for additional Class 3 and 4 antibodies (FIG. 19B-19D). We also confirmed inhibition of the spike RBD/ACE2 interaction by SL1111_20. These results provide evidence that the binding epitope of SL111_20 on spike RBD overlaps the Class 3 and 4 epitopes and potentially has some overlap with the ACE2 binding region.

### Example 8: Hydrogen/Deuterium Exchange Mass Spectrometry (HDX-MS)

### Sequencing (LC-MS/MS)

To identify peptic fragments of spike RDB protein, 154 pmoles of protein (2 μL) were mixed with 48 μL of phosphate buffered saline (PBS, pH 7.2) and then mixed with 50 μL of a quenching buffer (6.67 M Urea, 0.2 M TCEP, pH 3.0). The mixture (a total of 100 μL) was incubated at 6° C. for 3 min and injected into a Waters HDX-LC box (Waters) in which protein was digested by an online pepsin column (Waters Enzymate BEH pepsin column, 5 m, 2.1×30 mm) and the resulting peptic peptides were trapped and desalted on a Waters ACQUITY UPLC BEH C4 1.7 m VanGuard Pre-column (2.1×5 mm) at 100 μL/min Buffer A (0.1% formic acid in water) for 3 min. The digestion chamber was kept at 15° C. and the trap and analytical columns were at 0° C. The peptides were eluted with 3-33% Buffer B (0.1% formic acid in acetonitrile) between 0-6 min, 33-40% B between 6-6.5 min, and 40-85% B between 6.5-7 min. The eluted peptides were resolved a Waters ACQUITY UPLC Protein BEH C4 column (300 Å, 1.7 m, 1 mm×50 mm). MS/MS spectra were performed on a Thermo LTQ orbitrap Velos mass spectrometer. The peptides were ionized using electrospray ionization (ESI) with the source voltage=4.5 kV and S-lens RF level=60%. The capillary temperature was 275° C. and the source heater was at 80° C. The sheath gas flow was 10. Precursor ions were scanned between 350-1,800 m/z at 60,000 resolution with AGC 1×10⁶ (max ion fill time=500 ms). From the precursor scan, the top 10 most intense ions were selected for MS/MS with 180 s dynamic exclusion (10 ppm exclusion window, repeat count=1) and AGC 1×10⁴ (max ion fill time=100 ms). Ions with unassigned charge states were rejected for MS/MS. The normalized collision energy was 35%, with activation Q=0.25 for 10 msec.

MS/MS spectra were searched against a database consisting of spike RBD protein sequence in a FASTA format using the MaxQuant/Andromeda program (version 1.6.3.4) developed by the Cox Lab at the Max Planck Institute of Biochemistry. The digestion mode was “unspecific” and oxidation of methionine was set as a variable modification. The minimum peptide length for the unspecific search was 5. MaxQuant/Andromeda used 4.5 ppm for the main search peptide tolerance and 0.5 Da for MS/MS tolerance. The false discovery rate was 0.01. A list of peptides identified from the search was used as an exclusion list for the next round of LC-MS/MS and a total of three LC-MS/MS were performed for each protein.

### Hydrogen Deuterium Exchange

A 500 μL of PBS were dried using vacuum centrifugation, reconstituted with an equal volume of deuterium oxide (“D20 buffer”). Spike protein (15 μL) was mixed with the same volume of either the compound (59 μM in water) or water and pre-incubated on ice for at least 1 hour to ensure the complex formation. Ten minutes prior to the initiation of HDX reaction, 5 μL of the preincubated sample was transferred to a 0.5 ml tube and incubated at room temperature. HDX reaction was initiated by the addition of the D20 buffer (45 μL) to the 5 μL sample (90% D20 final). The reaction was quenched at 1 and 10 min after the initiation by the addition of the quench buffer (50 μL). The quenched sample was incubated at 6° C. for 3 min and the whole sample (100 μL) was injected into the Waters HDX-LC box and LC-MS was performed as described in the above section. For HDX samples, only MS1 spectra were recorded.

### Data Analysis

HDX-MS data were analyzed using Mass Spec Studio (version 2.4.0.3484) developed by the Schriemer lab at the University of Calgary. “Peptide.txt” and “evidence.txt” from the MaxQuant/Andromeda search results were used to generate the “identification” table for Mass Spec Studio using an in-house Python script. The raw files from the Thermo LTQ orbitrap Velos were converted to mzML files using ProteoWizard (version 3.0.20216, 64 bit). The default processing parameters were used except mass tolerance=15 ppm, total retention time width=0.15 min, XIC smoothing=Savitzky Golay Smoothing, and deconvolution method=centroid. All processed data were manually validated.

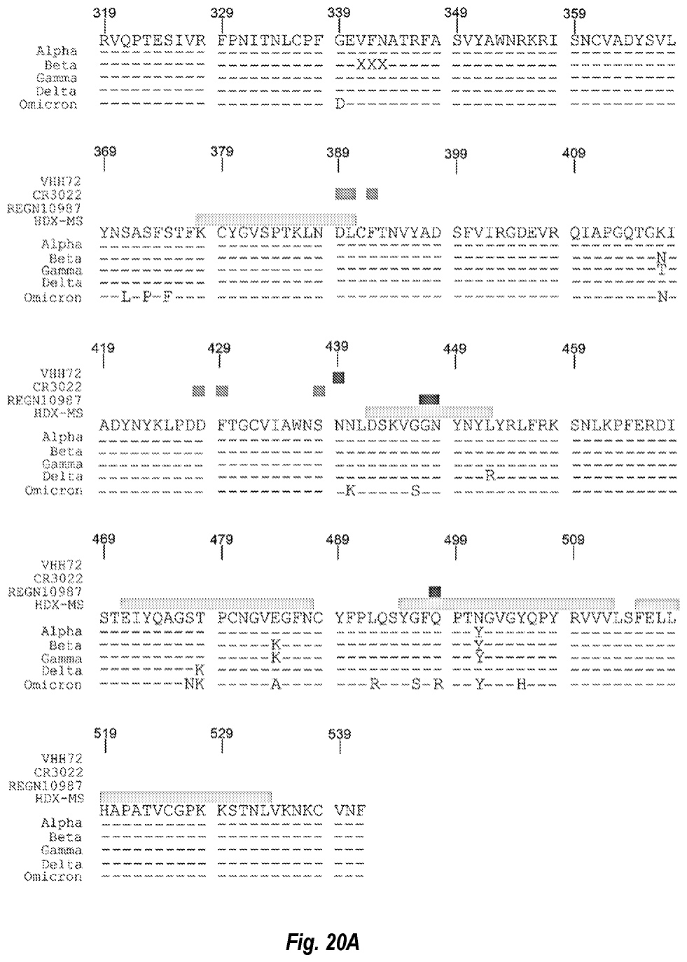

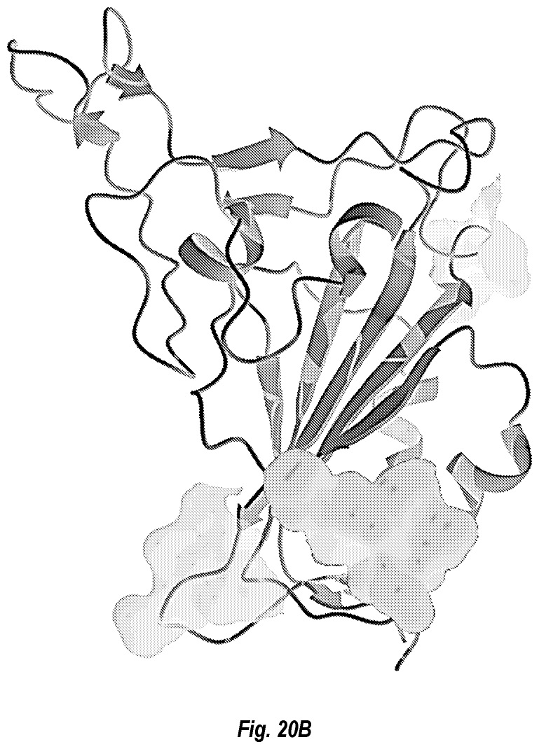

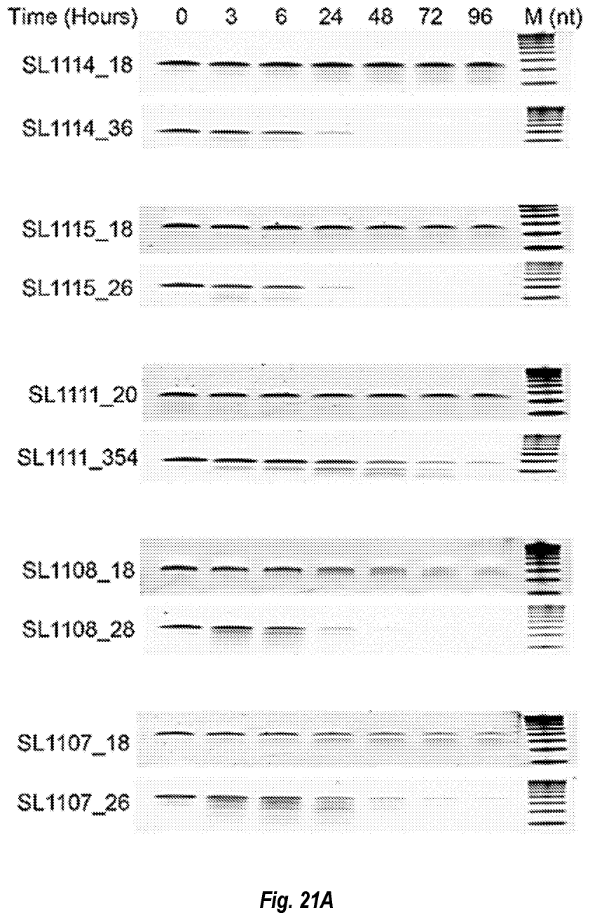

To complement the antibody competition experiments in Example 7 and gain additional insight regarding the potential binding epitope of SL1111_20 on spike RBD hydrogen-deuterium exchange mass spectrometry (HDX-MS) was performed. A total of 53 peptides encompassing 77% of the primary sequence of spike RBD were identified. Two regions could not be identified, amino acids 319-351 and 363-374, presumably due to glycosylation. When the spike RBD/SL1111_20 complex was analyzed, five peptides showed a significant change in deuteration compared to the free spike RBD indicating they are either directly protected or consequentially affected by the binding of SL111_20. These five peptides are shown in FIG. 20A and listed in Table 11 below. Three of the five peptides correspond to sites of antibodies inhibited from RBD binding by aptamer (where not shared with unaffected antibodies) (FIG. 20B). By mapping the known contact sites of all antibodies with RBD whose binding is unaffected by prior equilibration of RBD with SL1111_20 and those contact sites of antibodies inhibited from RBD binding by aptamer (where not shared with unaffected antibodies) it can be seen that SL1111_20 appears to disrupt binding at both the “rear” of the RBD, between the footprint of most Class 3 and 4 antibodies, and at the front of RBD, towards the base (FIG. 20). These sites are relatively conserved in VOCs, and so may not be under immune selection by antibody.

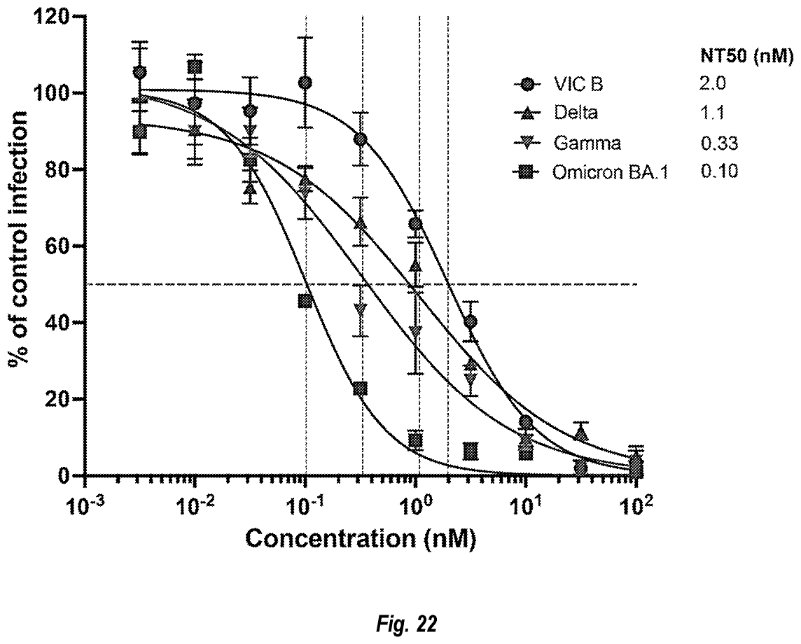

To test whether this conservation of binding to RBD variants, albeit distinct from that of known antibodies, was reflected in conserved antiviral effectiveness, neutralization assays with live Omicron VOC were undertaken. The results showed that SL1111_20 neutralized Omicron with at least equal potency to prototype (Clade B, VIC01) and Delta isolates (FIG. 22).

TABLE 11

| SEQ | Amino | |
|---|---|---|
| ID | Acid | |
| NO: | range | Sequence |
| 264 | 378-390 | KCYGVSPTKLNDL |
| 265 | 442-452 | DSKVGGNYNYL |
| 266 | 471-487 | EIYQAGSTPCNGVEGEN |
| 267 | 495-512 | YGFQPTNGVGYQPYRVVV |
| 268 | 515-533 | FELLHAPATVCGPKKSTNL |

### Example 9: Serum Stability Assay

Serum stability studies were performed in 90% pooled human sera (10% phosphate buffered saline) using 500 nM aptamer and samples were processed as described by Gupta et al, (J Biol Chem. 2014 Mar. 21; 289(12): 8706-8719). Briefly, aliquots were extracted with phenol-chloroform and concentrated using a YM-10 molecular weight cutoff filter (EMD Millipore). Digestion products for all studies were separated from full-length aptamer by PAGE using a 15% polyacrylamide gel containing 8 M urea. Electrophoresis, using a Tris borate buffer system, was performed for 20 minutes at 200 volts. To quantify bands, gels were stained with 2 μM SYBR® Gold nucleic acid stain (Molecular Probes) for 10 minutes. Images of stained aptamers were obtained using a Typhoon 9500 (G E Healthcare. Piscataway, NJ) and quantified with the ImageQuant TL software (with background subtraction). The fraction of intact aptamer was plotted as a function of time and fit to a one-phase exponential decay model to determine half-life. All aptamers used in these studies were purified via HPLC or gel purification and had a 3′-3′-linked dT cap and a 5′-hydroxyl group.

### Inhibitory SOMAmer Reagents Exhibit a Substantial Degree of Nuclease Resistance in Serum

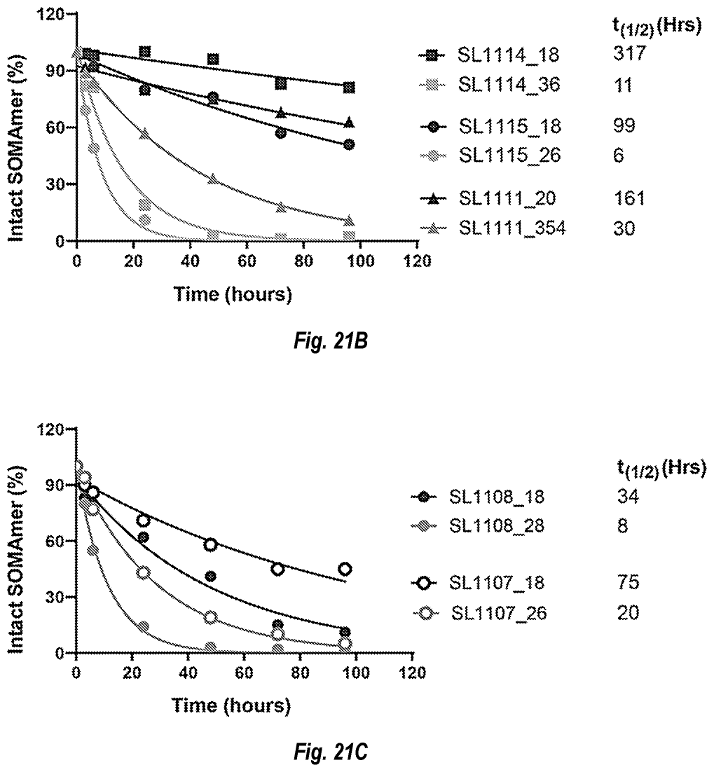

The strong neutralization potency of SL1111_20 against wild type and variant strains of SARS-CoV-2 makes it a highly attractive candidate for further development. To determine the metabolic stability of the nucleic acid-based reagents to nucleases present in human biological fluids, the in vitro half-life of SL1111_20 and the other lead candidates in pooled human serum was measured and compared to unmodified DNA analogs in which NapdU is replaced with dT (SL1107_18 and SL1108_18) and TyrdU and NapdC are replaced with dU and dC, respectively (SL1111_20, SL1114_18, SL1115_18). Purified SOMAmers were incubated with 90% pooled human serum at 37° C. and samples were removed for analysis at various time points up to 96 hours (FIG. 21A-21C). All reagents tested contained 3′-inverted dT to impede 3′-5′ exonuclease activity. Samples were analyzed by denaturing-PAGE and the percent intact aptamer was plotted as a function of time and fit to a one-phase exponential decay model to determine half-life. The two longest sequences, SL1108_18 (35-mer) and SL1107_18 (44-mer), which contain only NapdU modifications, had the shortest half-lives of 34 hours and 75 hours, respectively. The half-lives for the sequences with two modifications were longer and more varied, ranging from 99 hours for SL1115_18, 161 hours for SL1111_20 and 317 hours for SL1114_18. The presence of distinct degradation bands with all lead reagents indicates certain nucleotide positions are more sensitive to nuclease activity, suggesting half-lives could be further improved with supplementary nuclease stabilizing modifications such as 2′O-methyl and phosphorothioate. Not surprisingly, the unmodified analogs of the lead reagents were quickly degraded in human serum (half-life of 30 hours). These results are consistent with prior observations that shorter, heavily modified sequences tend to have the longest half-lives, while unmodified DNA is rapidly cleaved.

1. An aptamer that binds a SARS-CoV-2 protein, wherein the aptamer comprises the sequence 5′-GDRATRXTAHRXRTXHTRAXHIRXTXRRAXDDD-3′ (SEQ ID NO: 5) wherein,
A represents dA;
G represents dG;
C, T and X, each, independently, represent a C-5 modified pyrimidine nucleoside;
R is independently selected from a dA or dG;
H is independently selected from a dA, or a C-5 modified pyrimidine nucleoside; and
D is independently selected from a dA, dG or a C-5 modified pyrimidine nucleoside.

2. The aptamer of claim 1, wherein C independently represents the C-5 modified pyrimidine Nap-dC.

3. The aptamer of claim 1 or 2, wherein T independently represents the C-5 modified pyrimidine Tyr-dU.

4. The aptamer of any one of claims 1 to 3, wherein X independently represents a C-5 modified pyrimidine selected from a Nap-dC or Tyr-dU.

5. The aptamer of claim 4, wherein X independently represents the C-5 modified pyrimidine Nap-dC.

6. The aptamer of claim 4, wherein X independently represents the C-5 modified pyrimidine Tyr-dU

7. The aptamer of any one of claims 1 to 6, wherein H independently represents a C-5 modified pyrimidine selected from a Nap-dC or Tyr-dU.

8. The aptamer of claim 7, wherein H independently represents the C-5 modified pyrimidine Nap-dC.

9. The aptamer of claim 7, wherein H independently represents the C-5 modified pyrimidine Tyr-dU.

10. The aptamer of any one of claims 1 to 9, wherein D independently represents the C-5 modified pyrimidine Tyr-dU.

11. The aptamer of claim 1, wherein the aptamer comprises the sequence 5′-GGGATACTATGCGTCCGACCGCTCGGACGGA-3′ (SEQ ID NO: 4) wherein,
A represents dA;
G represents dG;
C represents Nap-dC; and
T represents Tyr-dU.

12. The aptamer of claim 1, wherein the aptamer comprises a sequence selected from SEQ ID NOs: 4, 6-20 and 28-122.

13. A heterodimeric aptamer that binds a SARS-CoV-2 protein, wherein the heterodimeric aptamer comprises a first aptamer comprising the sequence 5′-G G G A Y A p Y A Y G p G Y p p G A p p G p Y p G G A p G-3′ (SEQ ID NO: 7), a linkage covalently bonding the first aptamer to a second aptamer, and a second aptamer which binds a SARS-CoV-2 protein at a nonoverlapping binding site relative to the binding site of the first aptamer, wherein,
Y and p, each, independently, represent a C-5 modified pyrimidine nucleoside.

14. The heterodimeric aptamer of claim 13, wherein Y independently represents the C-5 modified pyrimidine 5-(p-hydroxyphenylethyl)-1-aminocarbonyl-2′-deoxyuridine (Tyr-dU).

15. The heterodimeric aptamer of claim 13, wherein p independently represents the C-5 modified pyrimidine 5-(1-Naphthylmethyl)aminocarbonyl-2′-deoxycytidine (Nap-dC).

16. The heterodimeric aptamer of any one of claims 13-15, wherein the first aptamer is linked 5′ of the second aptamer.

17. The heterodimeric aptamer of any one of claims 13-15, wherein the first aptamer is linked 3′ of the second aptamer.

18. The heterodimeric aptamer of any one of claims 13-17, wherein the linkage is a hexaethylene glycol (HEG) linkage.

19. The aptamer of any one of claims 1-12, wherein the aptamer is a first aptamer, further wherein the first aptamer is covalently linked to a second aptamer that binds a SARS-CoV-2 protein at a nonoverlapping binding site relative to the binding site of the first aptamer.

20. The aptamer of claim 19, wherein the first aptamer comprises a sequence selected from SEQ ID NOs: 4, 6-20 and 28-122.

21. The aptamer of claim 20, wherein the first aptamer comprises the nucleotide sequence of SEQ ID NO:4.

22. The aptamer of any one of claims 19-21, wherein the first aptamer is linked 5′ of the second aptamer.

23. The aptamer of any one of claims 19-21, wherein the first aptamer is linked 3′ of the second aptamer.

24. The aptamer of any one of claims 19 to 23, wherein the linkage is a hexaethylene glycol (HEG) linkage.

25. The aptamer of any one of claims 1 to 24, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.

26. The aptamer of any one of claims 1 to 25, wherein the SARS-CoV-2 protein comprises an amino acid sequence selected from SEQ ID NOs: 21-27 and 123-127.

27. An aptamer that binds a SARS-CoV-2 protein, wherein the aptamer comprises a sequence that is at least 80%, at least 85%, at least 90%, at least 95%, at least 97%, at least 99%, or 100% identical to a sequence selected from SEQ ID NOs: 4, 6-20 and 28-122.

28. The aptamer of any one of the preceding claims, wherein the aptamer has a dissociation constant (K_{d}) for the SARS-CoV-2 protein of 2 μM to 10 nM.

29. The aptamer of any one of the preceding claims, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

30. The aptamer of any one of the preceding claims, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

31. The aptamer of any one of the preceding claims, comprising at least one C-5 modified pyrimidine which is 5-(N-naphthylmethylcarboxyamide)-2′-deoxyuridine (NapdU).

32. The aptamer of any one of the preceding claims, wherein the aptamer comprises at least one 2′-O-methyl modified nucleotide.

33. The aptamer of any one of the preceding claims, wherein the aptamer is 24 to 100 nucleotides in length, or 30 to 60 nucleotides in length, or 28 to 60 nucleotides in length, or 28 to 50 nucleotides in length, or 28 to 40 nucleotides in length, or 40 to 50 nucleotides in length, or 28 to 32 nucleotides in length.

34. The aptamer of any one of the preceding claims, wherein the SARS-CoV-2 protein is the SARS-CoV-2 spike receptor binding domain (RBD).

35. The aptamer of claim 34, wherein the aptamer inhibits binding of the SARS-CoV-2 RBD to an angiotensin-converting enzyme 2 (ACE2) receptor.

36. The aptamer of any one of the preceding claims, wherein the aptamer inhibits SARS-CoV-2 viral cell membrane fusion with a host cell.

37. The aptamer of any one of the preceding claims, wherein the aptamer inhibits SARS-CoV-2 viral infection of human cells.

38. The aptamer of claim 37, wherein the IC₅₀ of the aptamer is less than 2.0E-9 (M).

39. A composition comprising the aptamer of any one of the preceding claims and a SARS-CoV-2 protein.

40. A pharmaceutical composition comprising a therapeutically effective amount of the aptamer of any one of claims 1 to 38 and at least one pharmaceutically acceptable excipient.

41. A method of treating or preventing a SARS-CoV-2 infection in a human, comprising administering a therapeutically effective amount of the aptamer of any one of claims 1 to 38 or the pharmaceutical composition of claim 40 to the human.

42. A method for detecting the presence of SARS-CoV-2 in a sample, comprising contacting the sample with the aptamer of any one of claims 1 to 38.

43. The method of claim 42, wherein the sample is in vitro.

44. A method for selecting an aptamer having binding affinity for a SARS-CoV-2 protein comprising: (a) contacting a candidate mixture with a SARS-CoV-2 protein, wherein the candidate mixture comprises modified nucleic acids in which one, several or all pyrimidines in at least one, or each, nucleic acid of the candidate mixture comprises a C-5 modified pyrimidine nucleoside; (b) exposing the candidate mixture to a slow off-rate enrichment process, wherein nucleic acids having a slow rate of dissociation from the target molecule relative to other nucleic acids in the candidate mixture bind the SARS-CoV-2 protein, forming nucleic acid-target molecule complexes; (c) partitioning slow off-rate nucleic acids from the candidate mixture; (d) amplifying the slow off-rate nucleic acids to yield a mixture of nucleic acids enriched in nucleic acid sequences that are capable of binding to the SARS-CoV-2 protein with a slow off-rate, whereby a slow off-rate aptamer to the SARS-CoV-2 protein molecule is selected.

45. The method of claim 44, wherein each nucleic acid is, independently, from 24 to 100 nucleotides in length, or from 30 to 60 nucleotides in length, or from 28 to 60 nucleotides in length, or from 40 to 50 nucleotides in length, or 28 nucleotides in length.

46. The method of claim 44 or 45, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

47. The method of any one of claims 44 to 46, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

48. The method of any one of claims 44 to 47, wherein at least one C-5 modified pyrimidine is 5-(N-naphthylmethylcarboxyamide)-2′-deoxyuridine (NapdU).

49. The method of any one of claims 44 to 48, wherein a plurality of nucleic acids in the mixture comprise at least one 2′-O-methyl modified nucleotide.

50. The method of any one of claims 44 to 49, wherein a plurality of nucleic acids in the mixture comprise a linker selected from a 3-carbon-spacer, a hexaethylene glycol linker, a polyethylene glycol linker, and any combination thereof.

51. The method of any one of claims 44 to 50, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.

52. A method for inhibiting binding of a SARS-CoV-2 protein to an angiotensin-converting enzyme 2 (ACE2) receptor, comprising contacting the SARS-CoV-2 protein with the aptamer of any one of claims 1-38.

53. The method of claim 52, wherein the SARS-CoV-2 protein is in a sample in vitro.

54. The method of claim 52, wherein the SARS-CoV-2 protein is in a human.

55. The method of any one of claims 52 to 54, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.

56. The method of any one of claims 52 to 55, wherein the SARS-CoV-2 protein is the SARS-CoV-2 RBD.

57. An aptamer that binds a SARS-CoV-2 protein, wherein the aptamer comprises the sequence 5′-DRHRRXWXWTGRXWXXTXDWDTXRARHR-3′ (SEQ ID NO: 253) or 5′-TRXDRXRXWXXWTWTTHRRXHTRRRNDB-3′ (SEQ ID NO: 255)
wherein
A is dA;
G is dG;
each C is independently, and for each occurrence, is a C-5 modified pyrimidine nucleoside;
each T is independently, and for each occurrence, is a C-5 modified pyrimidine nucleoside;
each R is independently, and for each occurrence, is dA or dG;
each W is independently, and for each occurrence, is dA or a C-5 modified pyrimidine nucleoside;
each X is independently, and for each occurrence, is a C-5 modified pyrimidine nucleoside;
each H is independently, and for each occurrence, is dA or a C-5 modified pyrimidine nucleoside;
each D is independently, and for each occurrence, is dA, dG or C-5 modified pyrimidine nucleoside;
each B is independently, and for each occurrence, is dG or a C-5 modified pyrimidine dU; and
each N is independently, and for each occurrence, is dA, dG or a C-5 modified pyrimidine nucleoside.

58. The aptamer of claim 57, wherein C is the C-5 modified pyrimidine Nap-dC.

59. The aptamer of claim 57 or 58, wherein T is the C-5 modified pyrimidine Tyr-dU.

60. The aptamer of any one of claims 57 to 59, wherein W is dA or the C-5 modified pyrimidine Tyr-dU.

61. The aptamer of any one of claims 57 to 60, wherein X is the C-5 modified pyrimidine Nap-dC or Tyr-dU.

62. The aptamer of any one of claims 57 to 61, wherein H is dA or the C-5 modified pyrimidine Nap-dC or Tyr-dU

63. The aptamer of any one of claims 57 to 62, wherein D is dA, dG or the C-5 modified pyrimidine Tyr-dU.

64. The aptamer of any one of claims 57 to 63, wherein B is dG or the C-5 modified pyrimidine Nap-dC or Tyr-dU.

65. The aptamer of any one of claims 57 to 64, wherein N is dA, dG or the C-5 modified pyrimidine Nap-dC or Tyr-dU.

66. The aptamer of claim 57 wherein the aptamer comprises the sequence 5′-GGCGGCACATGGCACTTCATATCGAGCG-3′ (SEQ ID NO: 252) or the sequence 5′-TGCAACGCACCTTATTCGGCTTGAATGT-3′ (SEQ ID NO: 254) wherein,
A represents dA;
G represents dG;
C represents Nap-dC; and
T represents Tyr-dU.

67. An aptamer that binds a SARS-CoV-2 protein, wherein the aptamer comprises the sequence 5′-TCDHCHXCXWRXTARXRARTRTCTRADTTGGAXXRRTCXTMXGG-3′ (SEQ ID NO: 257) or 5′-HXBWDWWRARTGTCTVNXTTGCAXTVGTGXBDXNN (SEQ ID NO: 259)-3′, wherein
A is dA;
G is dG;
C is dC;
each T is independently, and for each occurrence, is a C-5 modified pyrimidine nucleoside;
each R is independently, and for each occurrence is dA or dG;
each M is independently, and for each occurrence is dA or dC;
each W is independently, and for each occurrence is dA or a C-5 modified pyrimidine nucleoside;
X is independently, and for each occurrence is dC or a C-5 modified pyrimidine nucleoside;
H is independently, and for each occurrence is dA, dC or a C-5 modified pyrimidine nucleoside;
D is independently, and for each occurrence is dA, dG or a C-5 modified pyrimidine nucleoside;
V is independently, and for each occurrence is dA, dC or dG;
B is independently, and for each occurrence is dC, dG or a C-5 modified pyrimidine nucleoside; and
N is independently, and for each occurrence is dA, dC, dG or a C-5 modified pyrimidine nucleoside.

68. The aptamer of claim 67, wherein T is the C-5 modified pyrimidine NapdU.

69. The aptamer of claim 67 or 68, wherein R is dA or dG.

70. The aptamer of any one of claims 67 to 69, wherein M is dA or dC.

71. The aptamer of any one of claims 67 to 70, where W is dA or the C-5 modified pyrimidine Nap-dU.

72. The aptamer of any one of claims 67 to 71, wherein X is dC or the C-5 modified pyrimidine Nap-dU.

73. The aptamer of any one of claims 67 to 72, wherein H is dA, dC or the C-5 modified pyrimidine Nap-dU.

74. The aptamer of any one of claims 67 to 73, wherein D is dA, dG or the C-5 modified pyrimidine Nap-dU.

75. The aptamer of any one of claims 67 to 74, wherein V is dA, dC or dG.

76. The aptamer of any one of claims 67 to 75, wherein B is dC, dG or the C-5 modified pyrimidine Nap-dU.

77. The aptamer of any one of claims 67 to 76, wherein N is dA, dC, dG or the C-5 modified pyrimidine Nap-dU.

78. An aptamer that binds a SARS-CoV-2 protein, wherein the aptamer comprises a sequence that is at least 80%, at least 85%, at least 90%, at least 95%, at least 97%, at least 99%, or 100% identical to a sequence selected from SEQ ID NOs: 128-252, 254, 256 and 258.

79. The aptamer of any one of claims, 57 to 78, wherein each C-5 modified pyrimidine is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), N—(S-2-hydroxypropyl)-1-carboxamide-2′-deoxyuridine; 5-(N-ethylmorpholino)aminocarbonyl-2′-deoxyuridine (MOEdU); 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

80. The aptamer of any one of claims 57 to 78, wherein each C-5 modified pyrimidine is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine, 5-[N-(p-phenylbenzyl)carboxamide]-2′-deoxyuridine (PBndU), 5-[N-(4-phenoxybenzyl)carboxamide]-2′-deoxyuridine (POPdU), 5-[N-(3,3-diphenylpropyl)carboxamide]-2′-deoxyuridine (DPPdU), 5-[N-(3,4-dichlorophenylethyl) carboxamide]-2′-deoxyuridine (DCPE-dU), 5-[N-(2-((1,1′-biphenyl)-4-yl)ethyl)carboxamide]-2′-deoxyuridine (BPEdU), 5-(N-benzylcarboxamide)-2′-deoxycytidine (BndC); 5-(N-2-phenylethylcarboxamide)-2′-deoxycytidine (PEdC); 5-(N-3-phenylpropylcarboxamide)-2′-deoxycytidine (PPdC); 5-(N-1-naphthylmethylcarboxamide)-2′-deoxycytidine (NapdC); 5-(N-2-naphthylmethylcarboxamide)-2′-deoxycytidine (2NapdC); 5-(N-1-naphthyl-2-ethylcarboxamide)-2′-deoxycytidine (NEdC); 5-(N-2-naphthyl-2-ethylcarboxamide)-2′-deoxycytidine (2NEdC); 5-(N-tyrosylcarboxamide)-2′-deoxycytidine (TyrdC), 5-[N-(p-phenylbenzyl)carboxamide]-2′-deoxycytidine (PBndC), 5-[N-(4-phenoxybenzyl)carboxamide]-2′-deoxycytidine (POPdC), 5-[N-(3,3-diphenylpropyl)carboxamide]-2′-deoxycytidine (DPPdC), 5-[N-(3,4-dichlorophenylethyl) carboxamide]-2′-deoxycytidine (DCPE-dC), and 5-[N-(2-((1,1′-biphenyl)-4-yl)ethyl)carboxamide]-2′-deoxycytidine (BPEdC).

81. The aptamer of any one of claims 57 to 80, wherein the SARS-CoV-2 protein is selected from SARS-CoV-2 spike receptor binding domain (RBD), SARS-CoV-2 spike S1, spike S1 & S2 extracellular domain (ECD), and spike S1 & S2 ECD stable trimer.

82. The aptamer of any one of the preceding claims 57 to 81, wherein the aptamer inhibits binding of the SARS-CoV-2 RBD to an angiotensin-converting enzyme 2 (ACE2) receptor.

83. The aptamer of any one of the preceding claims 57 to 82, wherein the aptamer inhibits SARS-CoV-2 viral cell membrane fusion with a host cell.

84. The aptamer of any one of the preceding claims 57 to 83, wherein the aptamer inhibits SARS-CoV-2 viral infection of human cells.

85. A composition comprising the aptamer of any one of the preceding claims 57 to 84.

86. A pharmaceutical composition comprising a therapeutically effective amount of the aptamer of any one of claims 57 to 84 and at least one pharmaceutically acceptable excipient.

87. A method of treating or preventing a SARS-CoV-2 infection in a subject, comprising administering a therapeutically effective amount of the aptamer of any one of claims 57 to 84 or the pharmaceutical composition of claim 86 to the subject.

88. A method for detecting the presence of SARS-CoV-2 in a sample, comprising contacting the sample with the aptamer of any one of claims 57 to 84.

89. The method of claim 88, wherein the sample is in vitro.

90. The method of any one of claims 87 to 89, wherein the SARS-CoV-2 protein is selected from SARS-CoV-2 spike receptor binding domain (RBD), SARS-CoV-2 spike S1, spike S1 & S2 extracellular domain (ECD), spike S1 & S2 ECD stable trimer, spike S1 aspartic acid 614 to glycine mutant (D614G), spike RBD asparagine 501 to tyrosine mutant (N501Y), RBD glutamic acid 484 to lysine mutant (E484K),), a variant SARS-CoV-2 selected from B1.1.7 variant (Alpha), B1.351 variant (Beta), ECD P.1 variant (Gamma), B.1.617.2 variant (Delta), and B.1.1.529 variant (Omicron), and any combination thereof.

91. A method for inhibiting binding of a SARS-CoV-2 protein to an angiotensin-converting enzyme 2 (ACE2) receptor, comprising contacting the SARS-CoV-2 protein with the aptamer of any one of claims 57 to 84.

92. The method of claim 91, wherein the SARS-CoV-2 protein is in a sample in vitro.

93. The method of claim 91, wherein the SARS-CoV-2 protein is in vivo.
